SEQUENCE LISTING (58 sequences)

SEQ ID NO: 1 - protein, 163 aa, Toxoplasma gondii
MSDWDPVVKEWLVDTGYCCAGGIANAEDGVVFAAAADDDDGWSKLYKDDHEEDTIGEDGNACGKVSINEASTIKAAVDDGSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 2 - protein, 163 aa, Toxoplasma gondii
MSDWDPVVKEWLVDTGYCCAGGIANAEDGVVFAAAADDDDGWSKLYKEDHEEDTIGEDGNVNGKVTVNEASTIKAAVDDGSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 3 - protein, 196 aa, Toxoplasma gondii
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 4 - protein, 540 aa, Toxoplasma gondii
LWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETGQQTGTSGRRSSRSRGQSGREEGRREPQMVSLRIRVGGAPDCQVANEMQAAEVVQKCVSEADRIMHVFPEDVTPDVLVSRFRFAVPLYSGRIRHRSALLGQSRGYALLNYMDVLPPMLCDLFAAQSVVQPSMTRAIAKQLLELVAHLQALGVVHSDLKPENVLVDEAGNLFLADFDKAHKVGSRVRCSSLPHSLFADPQTVICRETTPDQEVTLAFSQDAWALGMLMYDTFCRNWPFMGMDYNNDSDTSNTIVLAGLLDRAAAVDTRAPIDWIGCDNPRVESEIKDLISRLLDIDPENRLDVLSFYMHSDFFNEGRNEANRSPPQRGAPILPLRESIV

SEQ ID NO: 5 - DNA, 26 bp, Artificial Sequence
  Synthetic primer
tcaactacgagcgttttaactgcaac

SEQ ID NO: 6 - DNA, 29 bp, Artificial Sequence
  Synthetic primer
ttggaatgatgggaacttaaaatcttccc

SEQ ID NO: 7 - DNA, 22 bp, Artificial Sequence
  Synthetic primer
caatcctgaaccgtgtgtgtct

SEQ ID NO: 8 - DNA, 25 bp, Artificial Sequence
  Synthetic primer
ttcatcaactgtcagagcagagaag

SEQ ID NO: 9 - protein, 588 aa, Toxoplasma gondii
MSKPFFPQVWVSCTGGVLNLRRHWLRVGNMSRAFAPTVLFVLLNVGGSLWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETGQQTGTSGRRSSRSRGQSGREEGRREPQMVSLRIRVGGAPDCQVANEMQAAEVVQKCVSEADRIMHVFPEDVTPDVLVSRFRFAVPLYSGRIRHRSALLGQSRGYALLNYMDVLPPMLCDLFAAQSVVQPSMTRAIAKQLLELVAHLQALGVVHSDLKPENVLVDEAGNLFLADFDKAHKVGSRVRCSSLPHSLFADPQTVICRETTPDQEVTLAFSQDAWALGMLMYDTFCRNWPFMGMDYNNDSDTSNTIVLAGLLDRAAAVDTRAPIDWIGCDNPRVESEIKDLISRLLDIDPENRLDVLSFYMHSDFFNEGRNEANRSPPQRGAPILPLRESIV

SEQ ID NO: 10 - protein, 218 aa, Toxoplasma gondii
LWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETECRTAFYDESHSKTVA

SEQ ID NO: 11 - protein, 196 aa, Unknown; identical to SEQ ID NO: 3
  Hypothetical protein

SEQ ID NO: 12 - protein, 196 aa, Unknown; identical to SEQ ID NO: 3
  Hypothetical protein

SEQ ID NO: 13 - protein, 196 aa, Shigella flexneri
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETEQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 14 - protein, 187 aa, Unknown
  Hypothetical protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGG

SEQ ID NO: 15 - protein, 184 aa, Unknown
  Hypothetical protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITVEDDHVVVDGNHMLAGQNLKFNVEVVAIREATEEELAHGHVHGAHDHHHDHDGCCGGHGHDHGHEHGGEGCCGG

SEQ ID NO: 16 - protein, 187 aa, Salmonella enterica
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITEVEDDHVVVDGNHMLAGQNLKFNVEVVAIREATEEELAHGHVHGAHDHHHDHGEDGCCGGHGHDHGHEHGGEGCCGG

SEQ ID NO: 17 - protein, 179 aa, Unknown
  Hypothetical protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEH

SEQ ID NO: 18 - protein, 163 aa, Unknown
  Hypothetical protein
MKVAKDLVVSLAYQVRTEDGVLVDESPVSAPLDYLHGHGSLISGLEALEGVGFDVVGANDAYGYDENVQRVPKDVFMGVDELQVGMRFLAETDQGPPVEITVDHVVVDGNHMLAGQNLKFNVEVVAIREATEEELAHGHVHGAHDHHDDHDGCCGGHHDHHEH

SEQ ID NO: 19 - protein, 185 aa, Dickeya dadantii
MKVAKDLVVSLAYQVRTEDGVLVDESPVSAPLDYLHGHGSLISGLEKALEGRDVGERFDVSVGANDAYGEYDENMVQRVPKDVFMGVDELQVGMRFLAETDQGPLPVEITEVQDEHVVVDGNHMLAGQNLKFNVEVVAIREATEEELAHGHVHGAHDHDHDGCCGGHGDHDGCCGGHDHDHSHEH

SEQ ID NO: 20 - protein, 551 aa, Toxoplasma gondii
MRLDAVYYLAQMASHRIPRCQATFRHFICWFLIFAAFAVSDFSNVHGSFLPDPHFLCHHRTLRFRRGYVPQLSPCPHASTHHVKSGSFSSKISFLEEAAQPPSPISSPESKTELLLSLSVGHYGKWMTDEDERGRRKVAASVSRLLRSERPKSISDRVQFLLDETVAPDTVFVVESLEGVPTAGVQVQAFRRGSPLASGSYNVLSEATPVPVPLPTSLLVRPKREVSQQSARMMNVVVAAVKGSEGHAAQVEEGKTGKAGVSSEPVAVRIALSARSSCEVEAEDECLSLARRYSNLMSKLPPFSSRDLLEKFGMVVPQLVGQLRGRPSLLRKQGDTSLLNFVQIMPLMACDLNQFRITDPDAVKFVVKRMIQLLALFGAAGLVHQDIKAENFLVSRQGRLYLADFDAVVRENDTIQCNKKLSLLFSPPEVLRCFFNTPEEKIALTQVVDSWSLGMAAWEVLCMSEPFDGMRFSEDDLYNMKVIAALPEGGDDTRALDWNRCRGRPPAPLRKAVECLLDRNAATRCKALDLFKSSPLFAEERPVPPEPSSSD

SEQ ID NO: 21 - DNA, 21 bp, Artificial Sequence
  Synthetic probe
tgccagcagccgcggtaattc

SEQ ID NO: 22 - DNA, 27 bp, Artificial Sequence
  Synthetic probe
ccaagtgctgtagtggatccctggtgg

SEQ ID NO: 23 - protein, 180 aa, Unknown
  Hypothetical protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHG

SEQ ID NO: 24 - protein, 157 aa, Unknown
  Hypothetical protein
MKVAKDLVVSLAYQVRTEDGVLVDESPVSAPLDYLHGHGSLIGLEALEGHVGDFDVVANDAYGQYDNLVQRVPKDVFMGVDELQVGMRFLAETDQGPVPVEITVDDVVVDGNHMLAGQNLFNVEVVAREATEELHGHVHGAHDHHCCGGHGHDHHEH

SEQ ID NO: 25 - protein, 184 aa, Edwardsiella ictaluri
MKVAKDLVVSLAYQVRTEDGVLVDESPVSAPLDYLHGHGSLIAGLEKALEGHVVGDRFDVHVPANDAYGQYDDNLVQRVPKDVFMGVDELQVGMRFLAETDQGPVPVEITEVGDDYVVVDGNHMLAGQNLNFNVEVVALREATAEELEHGHVHGAHDHHGECCGGHGNGEGECCGGHDHDHEHA

SEQ ID NO: 26 - protein, 158 aa, Unknown
  Hypothetical protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDH

SEQ ID NO: 27 - protein, 145 aa, Unknown
  Hypothetical protein
MKVAKDLVVSLAYQVRTEDGVLVDESPVSAPLDYLHGHGSLISGLETALEGEGFDVGNDAYGYDENLVQRVPKDVFMGVDELQVGMRFLAETDQGPVPVEITVEDDHVVVDGNHMLAGQNLFVEVAIREATEEELAHGHVHGHDH

SEQ ID NO: 28 - protein, 158 aa, Pectobacterium wasabiae
MKVAKDLVVSLAYQVRTEDGVLVDESPVSAPLDYLHGHGSLISGLETALEGREAGETFDVNIGSNDAYGNYDENLVQRVPKDVFMGVDELQVGMRFLAETDQGPVPVEITEVEDDHVVVDGNHMLAGQNLSFHVEVIAIREATEEELAHGHVHGEHDH

SEQ ID NO: 29 - protein, 158 aa, Unknown; identical to SEQ ID NO: 26
  Hypothetical protein

SEQ ID NO: 30 - protein, 141 aa, Unknown
  Hypothetical protein
MKVAKDLVVSLAYQVRTEDGVLVDESPVSAPLDYLHGHGSLISGLEALEGHEGDFDVVADYGYDENLVQRVPKDVFMGVDELVGMRFLATDQGPVPVEITAVEDHVVVDGNHMLAGQLFVEVVAIREATEELHGHVHGHDH

SEQ ID NO: 31 - protein, 158 aa, Pectobacterium wasabiae
MKVAKDLVVSLAYQVRTEDGVLVDESPVSAPLDYLHGHGSLISGLENALEGHEAGDRFDVRVSADDGYGSYDENLVQRVPKDVFMGVDELEVGMRFLADTDQGPVPVEITAVEDEHVVVDGNHMLAGQDLNFHVEVVAIREATAEELQHGHVHGEHDH

SEQ ID NO: 32 - protein, 152 aa, Unknown
  Hypothetical protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHV

SEQ ID NO: 33 - protein, 139 aa, Unknown
  Hypothetical protein
MKVAKDLVVSLAYQVRTEDGVLVDESPVSAPLDYLHGHGSLISGLEALEHGDKFVANDAYGQYDNLVQRVPKDVFMGVDELQVGMRFLAETDQGPVPVEITVEDHVVVDGNHMLAGQNLFNVEVVAIREATEELAHGHV

SEQ ID NO: 34 - protein, 152 aa, Pantoea ananatis
MKVAKDLVVSLAYQVRTEDGVLVDESPVSAPLDYLHGHGSLISGLENALENHVAGDKFEVHIPANDAYGQYDDNLVQRVPKDVFMGVDELQVGMRFLAETDQGPVPVEITEVEDEHVVVDGNHMLAGQNLNFNVEVVAIREATPEELAHGHV

SEQ ID NO: 35 - protein, 202 aa, Unknown
  Hypothetical protein
LWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGET

SEQ ID NO: 36 - protein, 192 aa, Unknown
  Hypothetical protein
LWVPRTLPVSAFQDVAPHHPCIGRPCDQVASVVAVPPANNLGFKREPSVGSPGHFSFLQAVGGVGGPPYAGDGSPLLASRLDWQFLQQWTDDNARDGFVAALRVTRSQILEDCNMEMVLLDRVSRPRITLSAVEPRTHRRRTLKLKIRSVAGTGGYNIVVKAFVIKSWFFRLGHWLSLGRQRNTARAPGGET

SEQ ID NO: 37 - protein, 202 aa, Toxoplasma gondii
LWVPRTLPVSAFQDDVAPHHPCIGRPCDQVASVVAVPPANNLGFKREPSVGSPGHFSFLQAVGGVGGPPYAGDGSPLLASRLDWQFLQQWQTDGDKNARDGFVAALPRVTRQSQILEDCNMEMVSLLDRVLSRNPRITLSAVEPRTHRRRTLKLKIRSVAGTGGYNIVVKAFVIKSWFFRLGHWLQSLGRQRNTARAPGGET

SEQ ID NO: 38 - protein, 430 aa, Unknown
  Hypothetical protein
QWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETGQQTGTSGRRSSRSRGQSGREEGRREPQMVSLRIRVGGAPDCQVANEMQAAEVVQKCVSEADRIMHVFPEDVTPDVLVSRFRFAVPLYSGRIRHRSALLGQSRGYALLNYMDVLPPMLCDLFAAQSVVQPSMTRAIAKQLLELVAHLQALGVVHSDLKPENVLVDEAGNLFLADFDKAHKVGSRVRCSSLPHSLFADPQTVICRETTPDQEVTLAFSQDAWALGMLMYDTFCRNWPFMGMDYNNDSDTSNTIVLAGLLDRAAAVDTRAPIDWIGCDNPRVESEIKDLISRLLDIDPENRLDVLSFYMHSDFFNEGR

SEQ ID NO: 39 - protein, 137 aa, Unknown
  Hypothetical protein
QWTDERSRTRSQLELDPTVEPRGTGNVVKALLRTPGGSPVLRIAVEAVPLFAVPLGIRRLLQNVLPMCDSTKLALAGVHDKENVLVGLFLDFDVCFPVCTPDAWGMDCWPFGMDNVLALDTRDWCELLDPRLMSFGR

SEQ ID NO: 40 - protein, 422 aa, Neospora caninum
QWMTADDEQGRTKLAESVSRLTRSQLLEGMSQKLMAVLDHAIPEGTDIVVEPVPGFQEGDHRVQAFRRGEALGTGSFNVVVKATPVPMPSGVSIVQRPKYALSPSLSTKFTRAVTTGAGPAAGPQAHATEGASHDAAFPPEGVALRITTARARSVMGELEAEDKCVSLVRSYESLKRKLPSGFSSEELLNKFGLAVPLMAGQIRGRPSLLRQEHNRSVVNFVQVLPVMVCDVQNVRSGDRHVATFLVKRMIQLGAFLGAAGIVHGDIKLENVLVSRTGQLFLTDFDALVRVNDLIVCGKKNTPEFSPPELVRCILDTPQEKIPATELVDAWSFGMAAWDVLCMTWPFAGMDFAARAIHNMNVLAALPEVGDDTRSLDWGRCKRRPPSAEVRAAVECLLDRNPRTRCTALDLFMSSPLFAAGR

SEQ ID NO: 41 - protein, 430 aa, Unknown; identical to SEQ ID NO: 38
  Hypothetical protein

SEQ ID NO: 42 - protein, 124 aa, Unknown
  Hypothetical protein
WTDEDERASRRSLLDETGYNASRGQARGGGGEPVRICVEKMPDLFVPGRRLLGLLNPMCDLPKLAAGVHDKENLVGLLADFDACLFPCTPLDWLGMCPFGMDDNVALDTRADWCRLLDRLSFER

SEQ ID NO: 43 - protein, 418 aa, Toxoplasma gondii
KWMTDEDERGRRKVAASVSRLLRSERPKSISDRAQFLLDEAVAPDTVFVVESLEGVPTAGVQVQAFRRGNPLASGSYNVLSEATPVPVPLPTSLLVRPERGEVPQQSARMMNVVVAAVKGSGGHAAHVEEGKTGKAGVSSEPVAVRIALSARSSCEVEAEDECLSLARKYSNLMSKLPPFSSRDLLEKFGMVVPQLVGQLRGRPSLLRKQGGNSLLNFVQIMPLMACDLNQFRITDPNAVKFVVKRMIQLLALFGAAGLVHQDIKTENFLVSRQGRLYLADFDAAVRENDTIQCNKKLSLLFSPPEVLRCFFNTPEEKIALTQVVDSWSLGMAAWEVLCMSEPFDGMRFSEDDLYNMKVIAALPEGGDDTRALDWNRCRGRPPAPLRKAVECLLDRNAATRCKALDLFKTSPLFAEER

SEQ ID NO: 44 - protein, 216 aa, Unknown
  Hypothetical protein
LWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETECRTAFYDESHSKT

SEQ ID NO: 45 - protein, 204 aa, Unknown
  Hypothetical protein
LWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETTS

SEQ ID NO: 46 - protein, 216 aa, Toxoplasma gondii
LWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETGQQTGTSGRRSSRS

SEQ ID NO: 47 - protein, 83 aa, Unknown
  Hypothetical protein
QWHTDEDENARDGFVAALSRVTRLSQILEDCNMEMVLLLDRVFSRSPRITLSAVEPRTHRRRTLKLKIRSVAGTGGYNIVVKA

SEQ ID NO: 48 - protein, 30 aa, Unknown
  Hypothetical protein
QWTDERSRTRSQLELDPTVEPRGTGNVVKA

SEQ ID NO: 49 - protein, 83 aa, Neospora caninum
QWMTADDEQGRTKLAESVSRLTRSQLLEGMSQKLMAVLDHAIPEGTDIVVEPVPGFQEGDHRVQAFRRGEALGTGSFNVVVKA

SEQ ID NO: 50 - protein, 113 aa, Unknown
  Hypothetical protein
LPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDC

SEQ ID NO: 51 - protein, 31 aa, Unknown
  Hypothetical protein
LVHHIPQAPGSFFLAVGDLFQDENRGVTLEC

SEQ ID NO: 52 - protein, 110 aa, Ascaris suum
LVVYEYLSSLSEHHRQILLPVQQILRKKGMLAAKKRILRSLPELGSDASFDFLHAVAISGDDGKNLVNDIYPNFHQVYQLLLNDRQENYRRGVVYYVAPHTPLAKVIESC

SEQ ID NO: 53 - protein, 250 aa, Unknown
  Hypothetical protein
MSKPFFPQVWVSCTGGVLNLRRHWLRVGNMSRAFAPTVLFVLLNVGGSLWVPRTLPVSAFQDAVAPHHPCIGRPCDQVASVVAVPPANNLGFKREPSVGSPGHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGET

SEQ ID NO: 54 - protein, 237 aa, Unknown
  Hypothetical protein
MSKPFFPQVVSCTGGVLNLRRHWLRVGNMSRFAPTVLFVLLNVGGLWVPRTLPVSAFQDVAPHHPCIGRPCDQVASVVAVPPANNLGFKREPSVGSPGHFSFLQAVGGVGGPPYAGDGSPLLASRLDWQFLQQWTDDNARDGFVAALRVTRSQILEDCNMEMVLLDRVSRPRITLSAVEPRTHRRRTLKLKIRSVAGTGGYNIVVKAFVIKSWFFRLGHWLSLGRQRNTARAPGGET

SEQ ID NO: 55 - protein, 250 aa, Toxoplasma gondii
MSKPFFPQVRVSCTGGVLNLRRHWLRVGNMSRSFAPTVLFVLLNVGGGLWVPRTLPVSAFQDDVAPHHPCIGRPCDQVASVVAVPPANNLGFKREPSVGSPGHFSFLQAVGGVGGPPYAGDGSPLLASRLDWQFLQQWQTDGDKNARDGFVAALPRVTRQSQILEDCNMEMVSLLDRVLSRNPRITLSAVEPRTHRRRTLKLKIRSVAGTGGYNIVVKAFVIKSWFFRLGHWLQSLGRQRNTARAPGGET

SEQ ID NO: 56 - protein, 452 aa, Unknown
  Hypothetical protein
SKISFLEEAAQPPSPISSPESKTELLLSLSVGHYGKWMTDEDERGRRKVAASVSRLLRSERPKSISDRVQFLLDETVAPDTVFVVESLEGVPTAGVQVQAFRRGSPLASGSYNVLSEATPVPVPLPTSLLVRPKREVSQQSARMMNVVVAAVKGSEGHAAQVEEGKTGKAGVSSEPVAVRIALSARSSCEVEAEDECLSLARRYSNLMSKLPPFSSRDLLEKFGMVVPQLVGQLRGRPSLLRKQGDTSLLNFVQIMPLMACDLNQFRITDPDAVKFVVKRMIQLLALFGAAGLVHQDIKAENFLVSRQGRLYLADFDAVVRENDTIQCNKKLSLLFSPPEVLRCFFNTPEEKIALTQVVDSWSLGMAAWEVLCMSEPFDGMRFSEDDLYNMKVIAALPEGGDDTRALDWNRCRGRPPAPLRKAVECLLDRNAATRCKALDLFKSSPLFAEER

SEQ ID NO: 57 - protein, 129 aa, Unknown
  Hypothetical protein
SFLPLLWTDEDERASRRSLLDETSGYNASRPEQQEEGVRICVECSARMPDLFVPGRRLLLLNPMCDLPKLAAGVHDKENLVGLLADFDCLFPCTPLDWLGMCPFGMDDNVALDTRADWCRLLDRLSFER

SEQ ID NO: 58 - protein, 465 aa, Toxoplasma gondii
GHFSFLQAVGGVGGPPYAGDGSPLLASRLDWQFLQQWHTDEDENARDGFVAALSRVTRLSQILEDCNMEMVLLLDRVFSRSPRITLSAVEPRTHRRRTLKLKIRSVAGTGGYNIVVKAFVIKSWFFRLGHWLGSLGRQRNTARAPGGETGQQTGTSGRRSSRSRGQSGREEGRREPQMVSLRIRVGGAPDCQVANEMQAAEVVQKCVSEADRIMHVFPEDVTPDVLVSRFRFAVPLYSGRIRHRSALLGQSRGYALLNYMDVLPPMLCDLFAAQSVVQPSMTRAIAKQLLELVAHLQALGVVHSDLKPENVLVDEAGNLFLADFDKAHKVGSRVRCSSLPHSLFADPQTVICRETTPDQEVTLAFSQDAWALGMLMYDTFCRNWPFMGMDYNNDSDTSNTIVLAGLLDRAAAVDTRAPIDWIGCDNPRVESEIKDLISRLLDIDPENRLDVLSFYMHSDFFNEGR

(19) United States
(12) Patent Application Publication
(10) Pub. No.: US 2013/0280297 A1
(43) Pub. Date: Oct. 24, 2013

(54) USE OF TOXOPLASMA GENE PRODUCTS TO PREVENT OR TREAT MICROBIAL INFECTIONS

(71) Applicants: Lori Neal, Madison, WI (US); Laura Knoll, Madison, WI (US); Erik Settles, Madison, WI (US); Lindsey Anne Moser, Madison, WI (US)

(72) Inventors: Lori Neal, Madison, WI (US); Laura Knoll, Madison, WI (US); Erik Settles, Madison, WI (US); Lindsey Anne Moser, Madison, WI (US)

(21) Appl. No.: 13/830,853

(22) Filed: Mar. 14, 2013

Related U.S. Application Data
(60) Provisional application No. 61/622,900, filed on Apr. 11, 2012.

(51) Int. Cl. C07K 14/45

(52) U.S. Cl. CPC C07K 14/45
USPC 424/209.1; 514/2.3; 424/93.2; 424/93.21; 514/2.4; 514/3.7; 514/4.6; 514/3.3; 514/4.4; 530/350

(57) ABSTRACT

The invention provides immunomodulatory proteins, e.g., useful to prevent, inhibit or treat microbial infection.

### CROSS-REFERENCE TO RELATED APPLICATIONS

This application claims the benefit of the filing date of U.S. Application Ser. No. 61/622,900, filed on Apr. 11, 2012, the disclosure of which is incorporated by reference herein.

### BACKGROUND

The discovery of potent antimicrobials dramatically reduced deaths from infectious diseases. In the 1960s, the US Surgeon General declared the “war on pestilence is won”. However, these medical advances are eroding as microbes have evolved a plethora of resistance mechanisms. Infectious disease is the second leading cause of death worldwide, and the third leading cause of death in the United States (Spellburg et al., 2008: WHO, 2004; Pinner al., 1996). The need to develop new antimicrobials with novel mechanisms is discussed in increasingly urgent tones (Boucher et al., 2009), and characterized as a crisis by the National Institute of Medicine (Microbial Threats to Health, 2003).

The very potency of many of the small molecules used as antibiotics has, in several ways, facilitated the evolution of resistant microbes and the reemergence of infectious diseases. Generally, a single antimicrobial is used at a time to treat infectious diseases. Diverse arrays of pathogens (viruses, bacteria, and parasites) have all demonstrated the ability to develop multiple sequential resistance mechanisms (Boucher et al., 2009). Due to fear of pandemic influenza, many countries have amassed large stockpiles of the relatively new antiviral oseltamivir (Tamiflu), but most seasonal influenza is already resistant to that drug (Moscona, 2009). When the Infectious Disease Society of America issued a policy statement on antimicrobial resistance they specifically suggested further research on immune enhancement strategies to combat antimicrobial resistance (Spellburg et al., 2008). While resistance to antibiotics is common, escape from vaccination strategies is rare. The effectiveness of vaccination is presumably because a successful immune response typically targets many epitopes of a pathogen, and uses diverse humoral and cellular mechanisms, some of which are innate. Unfortunately, the search for vaccines for malaria, HIV and many other pathogens remains unfulfilled.

There is an urgent need to identify and characterize new antimicrobial drugs for both treatment and control, for example, new antimicrobial drugs that are easy to administer and cost effective.

### SUMMARY OF THE INVENTION

Isolated Toxoplasma gondii (Tg or T. gondii) protein or related proteins, or vehicles to deliver that protein(s), can be used individually or in combination as a pre- and/or post exposure treatment against microbes. In one embodiment, the isolated T. gondii protein comprises Tg profilin (TgPRF) or a protein having at least 80%, 85%, 90%, 92%, 95%, 97%, 98%, or 99% amino acid sequence identity to SEQ ID NO:1 or SEQ ID NO:2. Mice stimulated with a single injection of purified TgPRF (recombinant PRF,rPRF) had a 3- to 4-log infection reduction in bacterial burden in the spleen and liver compared to control animals at 3 days post infection, did not develop clinical signs of disease, and survived for >30 days. TgPRF stimulation was associated with a rapid increase in the number of neutrophils and Ly6Chi monocytes in the spleen and production of CCL2, TNFα, IL-12 and IFNγ. TgPRF stimulation also reduced bacterial burden in mice deficient in T cells, NK cells, IL-12 and IFNγ, but did not provide any benefit in animals treated with neutrophil depleting antibody, suggesting its protective effect was primarily dependent on neutrophil recruitment. T. gondii-infected TLR11-deficient animals showed similar resistance to L. monocytogenes challenge, indicating that other T. gondii factors may function in similar, but TLR11-independent manners. This was surprising, as TLR11 is a receptor for TgPRF. Using TLR11-deleted mice, other active antimicrobial proteins were purified from a soluble protein fraction from T. gondii (STAg). Together these data show that TgPRF and related proteins, and other purified T. gondii factors, may be used as therapeutics to stimulate innate immunity against bacterial infections, as well as other microbial pathogens, e.g., through a receptor other than TLR11. Because humans likely lack functional TLR11, TgPRF and related proteins, and other T. gondii proteins, may be an effective immunotherapeutic in humans.

As described hereinbelow, a chronic T. gondii infection can prevent Plasmodium berghei ANKA-induced experimental cerebral malaria (ECM) in C57BL/6 mice. STAg, a soluble non-infectious extract from T. gondii, prevented ECM symptoms, blood brain barrier permeability, and mortality when administered up to two days after infection. STAg treatment of P. berghei-infected mice reduced parasite sequestration and T cell infiltration in the brain. STAg treatment during P. berghei infection increased serum levels of IL-12, MCP-1, IL-6, and IFN-γ within 14 hours after administration. However, STAg treatment reduced P. berghei-induced IFN-γ levels four days later. Using IL-12βR or IL-10 deficient mice, IL-12βR but not IL-10 was found to be essential for STAg treatment of ECM. Collectively, this data suggested that STAg-reduced T-cell sequestration and IFN-γ accumulation requires the IL-12 pathway but not IL-10. STAg was fractionated by anion exchange to identify proteins that prevented ECM. Fractions that contained T. gondii profilin, a known stimulator of IL-12, prevented ECM. Purified profilin prevented ECM, which suggests that profilin is sufficient to prevent ECM. Profilin activity against P. berghei is indicative that an agent is likely effective against other category B coccidian parasites such as Cryptosporidium parvum and Cyclospora cayatanensis.

In one embodiment, a composition of the invention may be employed to prevent, inhibit or treat a microbial infection. Thus, the present invention relates to immunomodulatory compositions and methods which employ native or recombinant isolated protein e.g., profilin, cyclophilin or ROP39 or a protein having at least 80%, 85%, 90%, 92%, 95%, 97%, 98%, or 99% amino acid sequence identity to SEQ ID NO:1 (profilin), SEQ ID NO:2 (profilin), SEQ ID NO:3 (cyclophilin), or SEQ ID NO:4 (Rop39), or a recombinant virus or recombinant cell, such as an attenuated or avirulent Toxoplasma, which expresses one or more recombinant gene products one or more of which is an immunomodulatory protein of the invention, or soluble extracts of those cells, or inactivated recombinant Toxoplasma, e.g., inactivated via chemical or heat treatment, which expresses one or more isolated protein(s), one of which is an immunomodulatory protein of the invention, e.g., profilin, ROP39 or cyclophilin (peptidyl-prolyl cis-trans isomerase B) or soluble extracts thereof. In one embodiment, the isolated protein is obtained from a recombinant bacterial cell.

The invention provides compositions and methods for beneficially modulating an immune response to a variety of different microbial pathogen infections, e.g., viral, bacterial, fungal or parasitic infections, in animals including avians and mammals. Thus, the compositions of the invention, for example, a single dose thereof, are broad spectrum immunotherapeutics and, as disclosed herein, may provide for prophylactic and/or therapeutic activity against a variety of diverse microbes. In one embodiment, the method includes administering to a mammal having or suspected of having a microbial pathogen infection, e.g., a mammal exposed to a microbial pathogen, a composition comprising an effective amount of a native or recombinant isolated protein e.g., profilin, cyclophilin or ROP39 or a protein having at least 80%, 85%, 90%, 92%, 95%, 97%, 98%, or 99% amino acid sequence identity to SEQ ID NO:1. SEQ ID NO:2, SEQ ID NO:3, or SEQ ID NO:4, or combinations thereof, or a recombinant virus or cell, such as an attenuated or avirulent Toxoplasma, which expresses one or more recombinant gene products at least one of which is an immunomodulatory protein of the invention, or soluble extracts of those cells, inactivated recombinant Toxoplasma, e.g., inactivated via chemical or heat treatment, which expresses one or more isolated protein(s), at least one of which is an immunomodulatory protein of the invention, or soluble extracts. In one embodiment, the composition or method employs isolated recombinant protein having at least 80%, 85%, 90%, 92%, 95%, 97%, 98%, or 99% amino acid sequence identity to SEQ ID NO:1, SEQ ID NO:2, SEQ ID NO:3, or SEQ ID NO:4, or a combination thereof. In one embodiment, the composition or method employs isolated recombinant protein having SEQ ID NO:1, SEQ ID NO:2, SEQ ID NO:3, or SEQ ID NO:4, or a combination thereof, for example SEQ ID NO:1 and SEQ ID NO:3, or SEQ ID NO:4 and SEQ ID NO:3.

In one embodiment, the invention provides a method to enhance neutrophil numbers or neutrophil migration in a mammal. The method includes administering to a mammal in need thereof a composition comprising an effective amount of an isolated protein having at least 80% amino acid sequence identity to SEQ ID NO:1, or a recombinant bacterial cell, recombinant eukaryotic cell or recombinant virus that expresses an isolated protein having at least 80% amino acid sequence identity to SEQ ID NO:1, or any combination thereof.

In one embodiment, the invention provides a composition comprising an amount of an isolated protein having at least 80% amino acid sequence identity to SEQ ID NO:1 effective to inhibit or prevent microbial pathogen infection or replication, or one or more symptoms or manifestations of the microbial pathogen infection or replication, in a TLR11^{-/-} mammal.

In one embodiment, the composition further comprises a pharmaceutically acceptable carrier. In one embodiment, the composition is administered orally, for instance, in a formulation suitable to deliver isolated protein(s). In another embodiment, the composition is administered through various other acceptable delivery routes, for example, through parenteral injection, intranasally, or via an intra-muscular injection. In one embodiment, the composition is administered to the mammal one or more times, at times including but not limited to 1 to 7 days, 1 to 3 weeks or about 1, 2, 3, 4 or more, e.g., up to 6, months, before the mammal is exposed to the pathogen. In one embodiment, the composition is administered to the mammal one or more times after exposure to the pathogen, e.g., at 1 hour, 6 hours, 12 hours, 1 day, 2 days, 4 days or more, e.g., up to about 2 weeks, after exposure. In one embodiment, the composition is administered to the mammal when the mammal is symptomatic. In one embodiment, the administration of a composition of the invention results in an increase in CD8⁺ T cells in the mammal. In one embodiment, the administration of a composition of the invention results in an increase in neutrophils and Ly6Chi monocytes in the mammal.

A protein for use in the compositions and methods of the invention may have at least 80%, 85%, 90%, 92%, 95%, 97%, 98%, or 99% amino acid identity over the complete sequence of SEQ ID NO:1, SEQ ID NO:2, SEQ ID NO:3 or SEQ ID NO:4, and the substituted residues may be conservative or non-conservative substitutions. For example, in one embodiment, a protein for use in the compositions and methods of the invention has up to 5% of the residues, e.g., 1, 2, 3, or 4 residues, underlined in FIG. 8 substituted with conservative substitutions, up to 10% of the bold italicized residues in FIG. 8 substituted with conservative substitutions, or up to 20% of the unmarked residues in FIG. 8 substituted, or any combination thereof, relative to SEQ ID NO:1 or SEQ ID NO:2. In one embodiment, a protein for use in the compositions and methods of the invention has up to 5% of the residues underlined in FIG. 8 substituted with conservative substitutions, up to 5%, e.g., 1 or 2, of the bold italicized residues in FIG. 8 substituted with conservative substitutions, or up to 20% of the unmarked residues in FIG. 8 substituted, or any combination thereof, relative to SEQ ID NO:1 or SEQ ID NO:2. In one embodiment, a protein for use in the compositions and methods of the invention has up to 10% of the residues underlined in FIG. 8 substituted with conservative substitutions and up to 40% of the unmarked residues in FIG. 8 substituted relative to SEQ ID NO:1 or SEQ ID NO:2. Whether a particular amino acid substitution results in a functional polypeptide can readily be determined by assaying the biological activity of the variant polypeptide by methods well known to the art.

In one embodiment, a protein for use in the compositions and methods of the invention has up to 5% of the residues, e.g., 1, 2, 3, 4, 5, 6, 7 or 8 residues, in SEQ ID NO:3 FIG. 17 substituted with conservative or nonconservative substitutions, up to 10% of the residues in SEQ ID NO:3 in FIG. 17 substituted with conservative or nonconservative substitutions, or up to 20% of the residues in SEQ ID NO:3 in FIG. 17 substituted, or any combination thereof. In one embodiment, the substitutions are at one or more positions marked with bold underlined residues in FIG. 17. In one embodiment, a protein for use in the compositions and methods of the invention has up to 5% to 10% of the residues in SEQ ID NO:3 in FIG. 17 substituted with conservative substitutions, up to 15% of the residues in SEQ ID NO:3 in FIG. 17 substituted with conservative substitutions, or up to 20% of the residues in SEQ ID NO:3 in FIG. 17 substituted, or any combination thereof, e.g., where the substitutions are at one or more positions marked with bold underlined residues in FIG. 17. In one embodiment, a protein for use in the compositions and methods of the invention has one or more of the residues marked by bold and underlining in SEQ ID NO:3 in FIG. 17 substituted with conservative substitutions and up to 20% of the unmarked residues in FIG. 17 substituted. Whether a particular amino acid substitution results in a functional polypeptide can readily be determined by assaying the biological activity of the variant polypeptide by methods well known to the art.

In one embodiment, a protein for use in the compositions and methods of the invention has up to 5% of the residues, e.g., 1, 2, 3, 4, 5, 6, 7, 8, 9, 10 or 11 residues, in SEQ ID NO:4, SEQ ID NO:9 or SEQ ID NO:10 in FIG. 18 substituted with conservative or nonconservative substitutions, up to 10% of the residues in SEQ ID NO:4, SEQ ID NO:9 or SEQ ID NO:10 in FIG. 18 substituted with conservative substitutions, or up to 20% of the residues in SEQ ID NO:4, SEQ ID NO:9 or SEQ ID NO:10 in FIG. 18 substituted, or any combination thereof. In one embodiment, the substitutions include those at one or more positions marked with bold underlined residues in FIG. 18. In one embodiment, a protein for use in the compositions and methods of the invention has up to 5% of the residues in SEQ ID NO:4, SEQ ID NO:9 or SEQ ID NO:10 in FIG. 18 substituted with conservative substitutions, and up to 5% of the residues in SEQ ID NO:4, SEQ ID NO:9 or SEQ ID NO:10 in FIG. 18 substituted with non-conservative substitutions, or up to 10% of the residues in SEQ ID NO:4, SEQ ID NO:9 or SEQ ID NO:10 in FIG. 18 substituted with conservative substitutions, and up to 10% of the residues in FIG. 18 substituted with nonconservative substitutions, or any combination thereof, e.g., where the substitutions are at one or more positions marked with bold underlined residues in FIG. 18. Whether a particular amino acid substitution results in a functional polypeptide can readily be determined by assaying the biological activity of the variant polypeptide by methods well known to the art.

In one embodiment, the use of a composition of the invention having one or more isolated gene products of Toxoplasma, such as profilin or ROP39, or corresponding gene products of other organisms, e.g., bacterial cyclophilin, may prevent, inhibit or treat heterologous (non-Toxoplasma) microbial infection, e.g., infection by a virus, bacterium, fungus or parasite, of an animal, such as an avian or a mammal including human and non-human mammals. In one embodiment, the composition comprises about 100 μg/mL to about 2000 μg/mL, e.g., about 150 to about 300 μg/mL. In one embodiment, the composition comprises about 5 mg to about 1000 mg, e.g., about 10 mg to about 50 mg, or about 1 μg to about 1000 μg, e.g., about 10 μg to about 100 μg or up to about 1000 μg, e.g., 200 μg, of protein per dose.

In one embodiment, the administration of a composition of the invention to avians or mammals provides for decreased bacterial colonization after exposure to bacterial infection, e.g., at least a 1 or 2 log drop in colony forming units relative to colony forming units in the absence of the administration of that composition or any other prophylactic or therapeutic agent.

In one embodiment, the administration of a composition of the invention to avians or mammals provides for enhanced survival, e.g., after exposure to influenza virus, including survival rates of at least 35% or greater, for instance, survival rates of 50%, 60%, 70%, 75%, 80%, 85%, 90% or greater, relative to survival rates in the absence of the administration of that composition or any other prophylactic or therapeutic agent. The compositions of the invention are useful prophylactically or therapeutically against seasonal flu and other viral infections.

Accordingly, in one embodiment, the invention provides compositions and methods for preventing and limiting influenza infection that may be more persistent and easier to develop than current methods, given that new vaccines would not need to be developed each season. Moreover, the use of the compositions of the invention may be less likely to result in resistant influenza strains than current antiviral approaches. Further, the composition may comprise combinations of the aforementioned compositions with influenza virus immunogens (antigens), e.g., HA and NA.

The isolated immunomodulatory protein of the invention, or recombinant virus or recombinant cells expressing that protein, may be useful in the treatment of antibiotic resistant infection because, as an immune modulator, treatment with this protein is not likely to engender resistance, in addition to the treatment of bacterial infections. Moreover, this protein may be administered to patients suffering from infections of unknown origin because of its potentially broad-spectrum effects, or patients suffering from infections with pan resistant gram-negative bacilli or multi-resistant gram-positive bacteria like methicillin resistant Staphylococcus aureus. In another embodiment, the isolated immunomodulatory protein of the invention may be employed as a vaccine adjuvant to improve response to immunogens. Further, because of the demonstrated effect of TgPRF on boosting neutrophil counts, this protein may boost neutrophil counts in patients suffering from neutropenia as a result of aplastic anemia, blood cancers, chemotherapy, hereditary disorders, radiation, various vitamin deficiencies, autoimmune diseases, or hemodialysis.

Because the induction of an adaptive immune response is not necessary for protection by the compositions of the invention, as disclosed herein, those compositions are likely to be effective in T cell deficient mammals, e.g., humans with HIV, autoimmune disorders, organ transplant recipients, and calcineurin inhibitor recipients, and the administration of those compositions is less likely to result in the development of drug resistance by the infecting microbe. Further, augmenting the innate immune system in immunocompetent or immunocompromised avians and mammals in combination with traditional antimicrobials may decrease the pressure of microbes to develop resistance.

The invention provides a method to augment an immune response in a mammal. The method includes administering to a mammal a composition comprising an immunogen for a microbial pathogen and an amount of an isolated protein having at least 80% amino acid sequence identity to SEQ ID NO:3, SEQ ID NO:1, or SEQ ID NO:4, or a combination thereof, or a recombinant bacterial cell, recombinant eukaryotic cell or recombinant virus that expresses an isolated protein having at least 80% amino acid sequence identity to SEQ ID NO:3, SEQ ID NO:1, or SEQ ID NO:4, or any combination thereof, effective to augment the immune response in the mammal to the immunogen. In one embodiment, the microbial pathogen is a bacterium, e.g., Listeria. In one embodiment, the microbial pathogen is a parasite, e.g., Plasmodium.

### BRIEF DESCRIPTION OF THE DRAWINGS

FIG. 1 shows the sequence of exemplary T. gondii profilins of the invention (SEQ ID NO:1 and SEQ ID NO:2).

FIG. 2 shows graphs of bacterial burden in spleen and liver, and weight, of wild-type animals treated with rPRF or PBS.

FIG. 3 shows graphs of bacterial burden in spleen and liver, and weight, of wild-type animals treated with rPRF or PBS.

FIG. 4 shows survival after rPRF treatment pre- or post-bacterial infection.

FIG. 5 shows graphs of bacterial burden in spleen and liver, and weight, of TLR11 knockout animals treated with STAg or PBS.

FIG. 6 shows graphs of bacterial burden in spleen and liver, and weight, of TLR11 knockout animals treated with rPRF or PBS.

FIG. 7 shows neutrophil flow cytometry data for wild-type mice treated with rPRF or PBS.

FIG. 8 provides an exemplary consensus sequence showing conserved residues (underlined) among other PRF proteins in lower eukaryotes, and showing residues found in TgPRF (bolded italics), which may be substituted as discussed herein.

FIG. 9 shows A) SDS-gel fractionation of STAg and STAg fractions 1-3 and B) percent survival over time for mice treated with two of the STAg fractions or PBS (Circle=PBS, Fraction 2=regular triangle and Fraction 3=inverted triangle) in a cerebral malaria (CM) model. For A), protein was separated and fractions were collected using HighQ anion exchange chromatograph using a 0-1M NaCl discontinuous gradient. Collected fractions were pooled and the pooled fractions represent the following salt elution conditions Fraction 1: Flow through or 0 M NaCl up to 0.34 M NaCl, Fraction 2: 0.34 M to 0.52 M NaCl, and Fraction δ: 0.52 M to 1 M. Pooled fractions were buffer changed to PBS and concentrated prior to injection. Mice were treated one day after PbA infection (1.7 μg). See the description for FIG. 10 for a description of CM model and treatment timing.

FIG. 10 shows that chronic T. gondii infection or a T. gondii extract prevents PbA-induced cerebral malaria (CM). (A and B) C57BL/6 mice inoculated with T. gondii (Pru+PbA, n=6) or media (PbA, n=7) were challenged with 1×10⁶ Plasmodium berghei ANKA (PbA) infected red blood cells (iRBC) 28 days later. The results of a representative experiment measuring percent survival (A) and parasitized RBCs (B) are shown. (C and D) C57BL/6 mice were inoculated with 1×10⁶ iRBC and treated with PBS or soluble T. gondii antigens (STAg) at the indicated times after PbA infection. The average of multiple experiments is shown. (A and C) Infected mice were monitored for CM symptoms and morbidity for 16 to 17 days after PbA infection. (B and D) Thin blood smears were prepared at the indicated times after PbA infection. The average percentage of iRBC to total RBC was determined after Giemsa stain. P value is determined as a comparison of PbA alone or PbA+PBS controls and is denoted as *<0.05.

FIG. 11 illustrates that STAg reduces PbA-induced brain pathology and parasite localization. C57BL/6 mice inoculated with 1×10⁶ iRBC or mice that were not inoculated were treated with PBS or STAg two days after infection. The percentage of iRBC was monitored by thin blood smears and Giemsa stain. Once the parasitemia exceeded 8%, mice were injected with Evans blue dye. After 1 hour, the mice were euthanized, perfused with saline and the brains were collected, weighed and photographed (A). Evans blue was extracted from the tissue using formamide and the concentration of Evans blue per gram of brain tissue was determined by absorbance (B). C) Brains from naïve or PbA infected mice were collected and genomic DNA was prepared. The number of PbA 18S and mouse albumin genomic DNA copies was determined by qPCR. The number of PbA copies was normalized to mouse albumin. In panels B and C, each symbol represents an individual mouse. Samples indicated with an asterisk represent statistical significance compared to PbA/PBS infected mice (P<0.05). The results of representative experiments are shown.

FIG. 12 shows that STAg reduces brain localized T cells. C57BL/6 mice inoculated with 1×10⁶ iRBC or were treated with STAg or PBS 1 (D) or 2 (A-D) days later. (A-B) Mice were euthanized, perfused and brain tissue was collected 6 days after PbA infection. Brain mononuclear cells were isolated and fluorescent conjugated antibodies were used to detect CD3⁺CD8⁺ or CD3⁺CD4⁺ cells. Representative CD4⁺ and CD8⁺ gates are shown (A). The total number of brain CD8⁺ (B) and CD4⁺ (C) cells was determined. Each symbol represents an individual mouse. D) Whole blood was collected at 6 days after infection and serum was prepared (n=3 per time point). IFN-γ cytokine levels were quantitated by cytokine bead array. Samples indicated with an asterisk represent statistical significance compared to bracketed samples (P<0.05). Representative experiments are shown.

FIG. 13 shows that STAg treatment increases serum cytokine levels. C57BL/6 mice inoculated with 1×10⁶ iRBC or mice that were not inoculated were treated with PBS or STAg 1 day later. Whole blood was collected at the indicated times after treatment and serum was prepared (n=3 per time point). IL-12p70, IFN-γ, MCP-1 and IL-6 cytokine levels were quantitated using a cytokine bead array. A representative experiment is shown.

FIG. 14 illustrates that IL-12 contributes to STAg-induced protection. C57BL/6 mice were inoculated with 1×10⁶ iRBC. WT, IL-12βR^{-/-} (C-D), or IL10^{-/-} (E) mice were treated with PBS, increasing amount of recombinant purified IL-12 (A and B) or STAg (C-E) 1 day later. Infected mice were monitored for CM symptoms and morbidity (A, C, E). Thin blood smears were prepared at 6 days after PbA infection (B and C). The average percentage of iRBC to total RBC was determined after giemsa stain. Two independent experiments are shown.

FIG. 15 shows the results of STAg fractionation. STAg was fractionated by anion exchange chromatography. Fractions were collected, pooled, and desalted into PBS. Pooled fractions were subjected to SDS-PAGE, silver stain or western blot analysis with anti-PRF (A). TLR11^{-/-} C57BL/6 mice were inoculated with 1×10⁶ iRBC. Infected mice were treated with PBS or fractionated STAg 1 day after PbA infection. Infected mice were monitored for CM symptoms and morbidity (B). A representative experiment is shown.

FIG. 16 illustrates that profilin (PRF) is sufficient to prevent PbA-induced CM. WT T. gondii or T. gondii expressing myc-tagged PRF (^{myc}PRF) under the expression of the tetracycline sensitive promoter was propagated in the presence and absence of anhydrotetracycline (ATET) for 4 days. STAg was prepared and subjected to western blot analysis with anti-PRF (A). WT C57BL/6 mice were inoculated with 1×10⁶ iRBC. Infected mice were treated with PBS, ^{myc}PRF STAg, ^{myc}PRF STAg+ATET (PRFKO STAg), recombinant purified ^{HIS6}PRF (B and C). Infected mice were monitored for CM symptoms and morbidity (B). Thin blood smears were prepared at 6 days after PbA infection (C). The results of two independent experiments are shown. Statistical significance was determined by a two-tailed student t-test. *=p<0.05. The results of two independent experiments are shown.

FIG. 17 shows the sequence of an exemplary cyclophilin protein (E. coli cyclophilin; SEQ ID NO:3) (A), a sequence shown with exemplary positions for substitution (underlined) (B), and alignments with related cyclophilins (C).

FIG. 18A illustrates the sequence of an exemplary T. gondii ROP39 protein (SEQ ID NO:4) that is expressed in T. gondii and its precursor protein (SEQ ID NO:9) which has an N-terminal sequence that is longer relative to SEQ ID NO:4, and a spliced variant (SEQ ID NO:10), and a homolog, Rop20 (SEQ ID NO:20). Other homologs include phosphorylase B kinase gamma, e.g., human phosphorylase B kinase gamma.

FIG. 18B shown alignments of T. gondii ROP39 with structurally related proteins.

FIG. 19 shows data for cyclophilin treated mice.

FIG. 20 illustrates that chronic T. gondii infection or stimulation with STAg reduces bacterial burden during L. monocytogenes infection. (A-B) Mice were infected i.p. with 250 tachyzoites of T. gondii to establish a chronic infection. On day 36, surviving T. gondii infected and uninfected mice were infected with about 6×10⁴ CFU of L. monocytogenes (n=6/group). (C-D) Naïve mice were stimulated i.v. with 1 μL of STAg or PBS 24 hours prior infection with L. monocytogenes (n=3/group). Bacterial burdens of the spleen and liver (A, C) and weight loss (B, D) were determined 72 hours later. Experiment with T. gondii infection was repeated one additional time. Experiments with STAg were repeated more than 10 times in C57BL/6 and NJ mouse strains.

FIG. 21 shows that stimulation with T. gondii profilin is sufficient to confer resistance to L. monocytogenes. Mice were stimulated i.v. with 100 ng of recombinant T. gondii profilin (rPRF) or PBS 4 hours prior infection with about 6×10⁴ CFU of L. monocytogenes. Bacterial burdens of the spleen and liver (A) and weight loss (B) were determined 72 hours later (n=3/group). Experiments were repeated more than three times. (C) Survival of L. monocytogenes infected mice stimulated with rPRF 4 hours prior to or post L. monocytogenes infection (n=8/group).

FIG. 22 illustrates that T. gondii profilin induces production of IL-12, MCP-1, IFN-γ, and TNF-α. Serum was collected from mice 2 hours (A-D) or 24 hours (E-H) following i.v. stimulation with 100 ng rPRF or PBS and assayed for cytokine levels by cytometric bead array.

FIG. 23 shows data for IL-12, IFN-γ, T/NK Cells (A-B) in IL-12β1 deficient (IL-12Rβ1^{-/-}) mice stimulated i.v. with 100 ng rPRF or PBS 4 hours prior infection with about 1×10⁴ CFU of L. monocytogenes (n=4-5/group). (C-D) IFN-γ deficient (IFN-γ^{-/-}) mice were stimulated i.v. with 100 ng rPRF or PBS 4 or 24 hours prior to infection with about 200 CFU of L. monocytogenes. The averages from three pooled experiments (each n=3-7/group) are shown. (E) Survival of IFN-γ^{-/-} mice (n=6/group) stimulated with 100 ng rPRF or PBS 4 hours prior to infection. (F-G) Rag1 deficient (Rag1^{-/-}) mice depleted with anti-NK1.1 MAb PK136 were stimulated i.v. with 100 ng rPRF or PBS 4 hours prior infection with about 8×10⁴ CFU of L. monocytogenes (n=4/group). The experiment was repeated one additional time. For all experiments bacterial burdens of the spleen and liver (A, C, F) and weight loss (B, D, G) were determined 72 hours later.

FIG. 24 illustrates that Ly6C^{hi} CCR2⁺ inflammatory monocytes and Ly6C^{int} Ly6G⁺ neutrophils are recruited to the blood and spleen in response to T. gondii profilin. Mice (n=4/group) were stimulated i.v. with 100 ng rPRF or PBS then blood and spleen cells were collected 4 hours later and stained for CD11b, Ly6C, Ly6G, and CCR2. Analysis was conducted on singlet, live cells and representative dot plots are shown for each analysis. Percentages of CD11b⁺ Ly6C^{hi} monocytes (elliptical gate) in the blood (A) and spleens (B). Percentages of Ly6C^{int} Ly6G⁺ neutrophils (square gate) in the blood (C) and spleens (D). (E) Expression of CCR2 on Ly6C^{hi} and Ly6G⁺ populations. Experiments were repeated more than three times.

FIG. 25 shows that CCR2-dependent recruitment of Ly6C⁺ cells, but not recruitment of Ly6G⁺ cells, is essential for T. gondii profilin-induced protection against L. monocytogenes. (A-B) CCR2 deficient (CCR2^{-/-}) mice were stimulated i.v. with 100 ng rPRF or PBS 4 hours prior infection with about 8×10³ CFU of L. monocytogenes (n=4-5/group). (C-D) Mice depleted with anti-Gr-1 (Ly6c/Ly6G) MAb RB6-8C5 were stimulated i.v. with 100 ng rPRF or PBS 4 hours prior infection with about 200 CFU of L. monocytogenes (n=4/group). (E-F) Mice depleted with anti-Ly6G MAb 1A8 were stimulated i.v. with 100 ng rPRF or PBS 4 hours prior infection with about 8×10³ CFU of L. monocytogenes (n=4/group). Bacterial burdens of the spleen and liver (A, C, E) and weight loss (B, D, F) were determined 72 hours later. All experiments were repeated at least three times.

FIG. 26 shows data for cyclophilin stimulate human PBMCs. A) Fold change in gene expression of human PBMCs stimulated with cyclophilin for 4 or 22 hours. B) Fold change in gene expression of human PBMCs stimulated with cyclophilin for 4 or 22 hours.

FIG. 27 provides flow cytometry data showing the recruitment of neutrophils and of inflammatory monocytes in response to rPRF.

FIG. 28 shows data for TLR11−/− mice infected with 2500 tachyzoites of T. gondii. To ensure survival, T. gondii-infected and control uninfected mice were all fed a diet containing sulfadiazine (1,365 ppm) and trimethoprim (275 ppm) (TD.06596, Harlan Teklad, Madison, Wis.) from days 9 through 14 post T. gondii infection, then returned to a normal diet on day 15 through the duration of the experiment. Mice were infected with 4×104 Listeria monocytogenes on day 38 post T. gondii infection and bacterial burdens in the spleen (A) and liver (B) and weight loss (C) were determined 72 hours later. TLR11−/− mice chronically infected with T. gondii had significantly reduced bacterial burdens in the spleen (p<0.0001) (A) and liver (p<0.0001) (B). T. gondii infected mice also loss significantly less weight than uninfected animals (p=0.0001) (C).

FIG. 29 shows data for TLR11−/− mice that were injected with STAg via i.v. route, then blood (A) and spleens (B) were collected 4 hours later an analyzed by flow cytometry. Inflammatory monocytes were identified as CD11b+ Ly6Chi and neutrophils were identified as Ly6Cint Ly6G+. neutrophils during infection. To determine if STAg stimulation promoted recruitment of Ly6Chi monocytes or neutrophils, TLR11−/− mice were stimulated with STAg or PBS via the i.v. route then analyzed blood and spleens by flow cytometry 4 hours later. An approximate 4-fold increase was observed in the percentage of CD11b+ Ly6Chi monocytes (p=0.0024) and an approximate 2.25-fold increase of Ly6Cint Ly6G+ neutrophils in blood (p=0.0022) in the blood of STAg treated mice (A). An approximate 2.5-fold increase was observed in the percentage of CD11b+ Ly6Chi monocytes (p=0.0049) and an approximate 3-fold increase of Ly6Cint Ly6G+ neutrophils in the spleens (p=0.0049) (B).

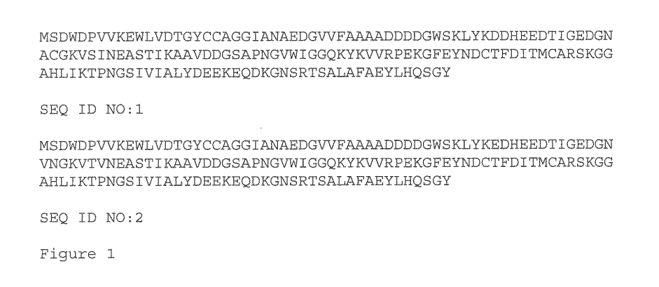

### DETAILED DESCRIPTION OF THE INVENTION

### Definitions

As used herein, the terms “isolated and/or purified” refer to in vitro preparation, isolation and/or purification of a microbial strain, cell or protein, so that it is not associated with and/or is substantially purified from in vitro or in vivo substances. An isolated strain or cell preparation of the invention is generally obtained by in vitro culture and propagation. A “recombinant” protein is one expressed using recombinant DNA techniques and a “recombinant” strain or cell is one which has been manipulated in vitro, e.g., using recombinant DNA techniques to introduce changes to the host genome. For example, a “recombinant” strain or cell of the invention may be one which has been manipulated in vitro so as to contain an insertion and/or deletion of DNA in the genome, e.g., chromosome, of the strain or cell relative to the genome, e.g., chromosome, of the parent strain or cell from which the recombinant strain or cell was obtained (e.g., “wild-type” strain). In one embodiment, an insertion in the recombinant strain is stable, e.g., the insertion and its corresponding phenotype do not revert to wild-type after numerous passages. Included within the scope of the phrase “recombinant strain” is one which, through homologous recombination, includes a gene which contains a mutation that results in the inactivation of the protein in or reduced expression of the gene, e.g., results in a polypeptide having reduced or lacking biological activity or so that the polypeptide is not expressed, relative to a corresponding wild-type strain that does not include the recombined gene.

A “soluble extract” as used herein, includes soluble preparations of lysed cells or subcellular fractions thereof, that include components of any Toxoplasma strain, components such as one or more proteins, which may be prepared by any method. For instance, a soluble extract of Toxoplasma may be prepared by subjecting isolated Toxoplasma to sonication or a French press, followed by removal of cellular debris by centrifugation or other methods known to the art.

As used herein, an “attenuated” strain means a strain, the inoculation of which to a susceptible mammal, results in reduced (mild) symptoms or manifestations of Toxoplasma infection.

As used herein, an Aavirulent@ strain means a strain, the inoculation of which to a susceptible mammal, results in no clinical manifestations of Toxoplasma infection.

The term “isolated polypeptide” means a protein encoded by cDNA, recombinant RNA, a synthetic nucleic acid or any other nucleic acid, or some combination thereof, which by virtue of its origin the “isolated polypeptide” (1) is not associated with proteins found in nature, (2) is free of other proteins from the same source, (3) is expressed by a cell from a different species, or (4) does not occur in nature.

The term “operably linked” referred to herein refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

The term “control sequence” referred to herein refers to polynucleotide sequences which are necessary to effect the expression of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequences. The term “control sequences” is intended to include, at a minimum, all components whose presence is necessary for expression, and can also include additional components whose presence is advantageous, for example, leader sequences and fusion partner sequences.

The term “nucleic acid sequence” as referred to herein means a polymeric form of nucleotides of at least about 7 bases in length, either ribonucleotides or deoxynucleotides or a modified form of either type of nucleotide. The term includes single and double stranded forms of DNA.

As applied to polypeptides, the term “substantial identity” means that two peptide sequences, when optimally aligned, share at least 80 percent sequence identity, e.g., at least 90, 95 or 99, percent sequence identity. In one embodiment, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, glutamic-aspartic, and asparagine-glutamine. Other conservative substitutions may be those within groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe. Yet other groups are hydrophobic amino acids and polar amino acids (the latter including neutral hydrophilic, acidic and basic residues).

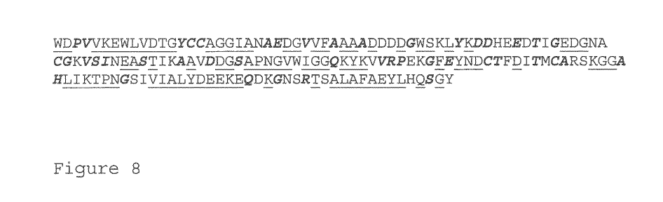

For example, in one embodiment, a protein or recombinant nucleic acid molecule encoding the protein for use in the compositions and methods of the invention has up to 5% of the residues, e.g., 1, 2, 3, or 4 residues substituted, underlined in FIG. 8 substituted with conservative substitutions, up to 10% of the bold italicized residues in FIG. 8 substituted with conservative substitutions, and up to 20% of the unmarked residues in FIG. 8 substituted, or any combination thereof, relative to SEQ ID NO:1 or SEQ ID NO:2. In one embodiment, a protein for use in the compositions and methods of the invention has up to 5% of the residues underlined in FIG. 8 substituted with conservative substitutions, up to 5%, e.g., 1 or 2, of the bold italicized residues in FIG. 8 substituted with conservative substitutions, and up to 20% of the unmarked residues in FIG. 8 substituted, or any combination thereof, relative to SEQ ID NO:1 or SEQ ID NO:2. In one embodiment, a protein for use in the compositions and methods of the invention has up to 10% of the residues underlined in FIG. 8 substituted with conservative substitutions and up to 40% of the unmarked residues in FIG. 8 substituted relative to SEQ ID NO:1 or SEQ ID NO:2. Whether a particular amino acid substitution results in a functional polypeptide can readily be determined by assaying the biological activity of the variant polypeptide by methods well known to the art. For example, a protein may have at least 80%, 85%, 90%, 92%, 95%, 97%, 98%, or 99% amino acid identity over the complete sequence of SEQ ID NO:1 or SEQ ID NO:2, and the substituted residues may be conservative or non-conservative substitutions.

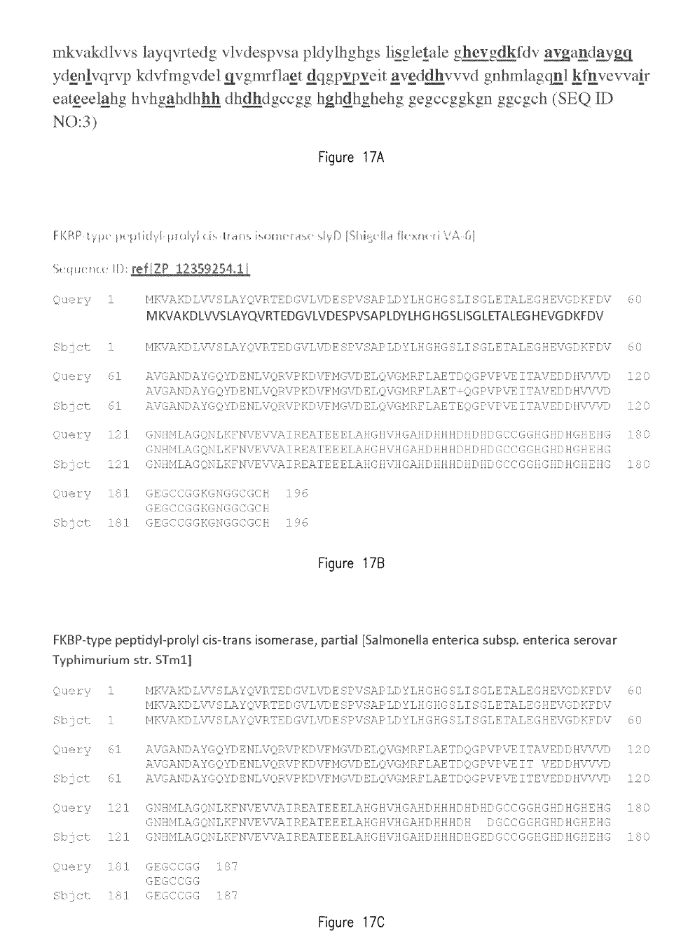

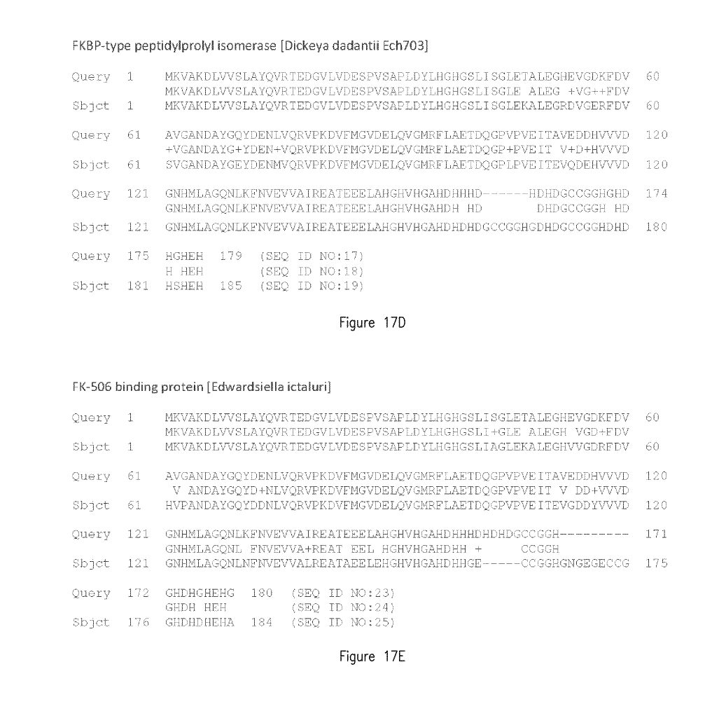

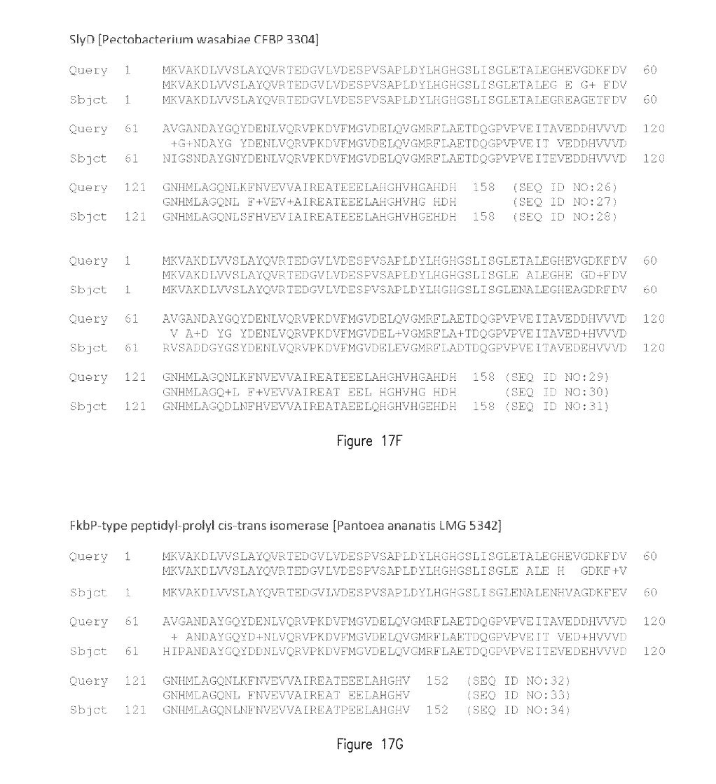

For example, in one embodiment, a protein for use in the compositions and methods of the invention has up to 5% of the residues, e.g., 1, 2, 3, 4, 5, 6, 7 or 8 residues, in SEQ ID NO:3 FIG. 17 substituted with conservative or nonconservative substitutions, up to 10% of the residues in SEQ ID NO:3 in FIG. 17 substituted with conservative or nonconservative substitutions, or up to 20% of the residues in SEQ ID NO:3 in FIG. 17 substituted, or any combination thereof. In one embodiment, the substitutions are at one or more positions marked with bold underlined residues in FIG. 17. In one embodiment, a protein for use in the compositions and methods of the invention has up to 5% to 10% of the residues in SEQ ID NO:3 in FIG. 17 substituted with conservative substitutions, up to 15% of the residues in SEQ ID NO:3 in FIG. 17 substituted with conservative substitutions, or up to 20% of the residues in SEQ ID NO:3 in FIG. 17 substituted, or any combination thereof, e.g., where the substitutions are at one or more positions marked with bold underlined residues in FIG. 17. In one embodiment, a protein for use in the compositions and methods of the invention has one or more of the residues marked by bold and underlining in SEQ ID NO:3 in FIG. 17 substituted with conservative substitutions and up to 20% of the unmarked residues in FIG. 17 substituted. Whether a particular amino acid substitution results in a functional polypeptide can readily be determined by assaying the biological activity of the variant polypeptide by methods well known to the art.

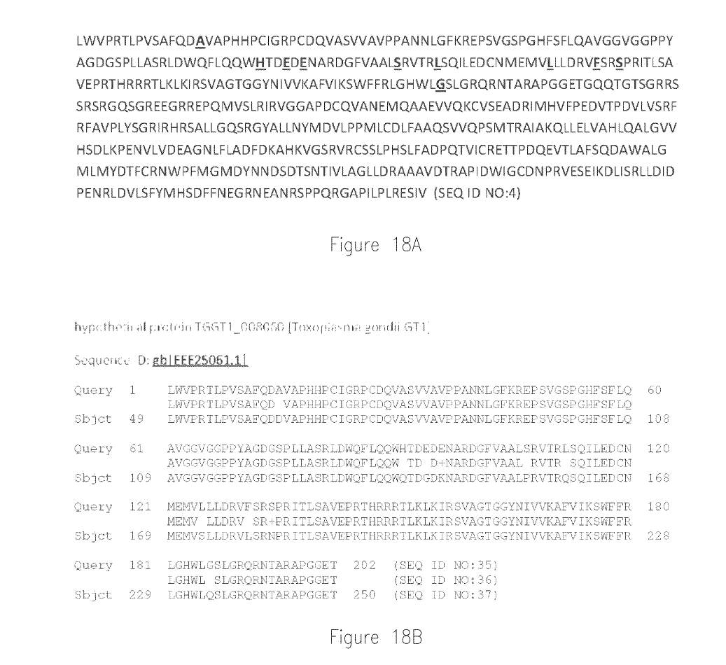

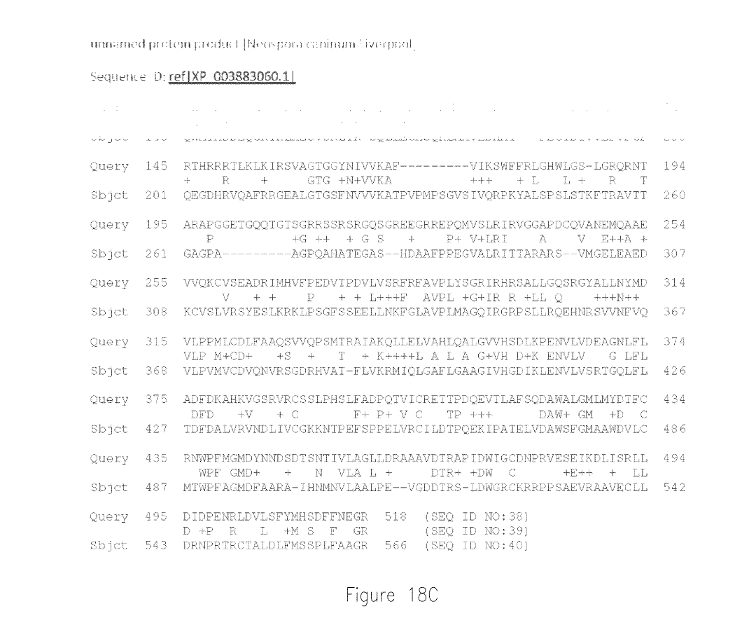

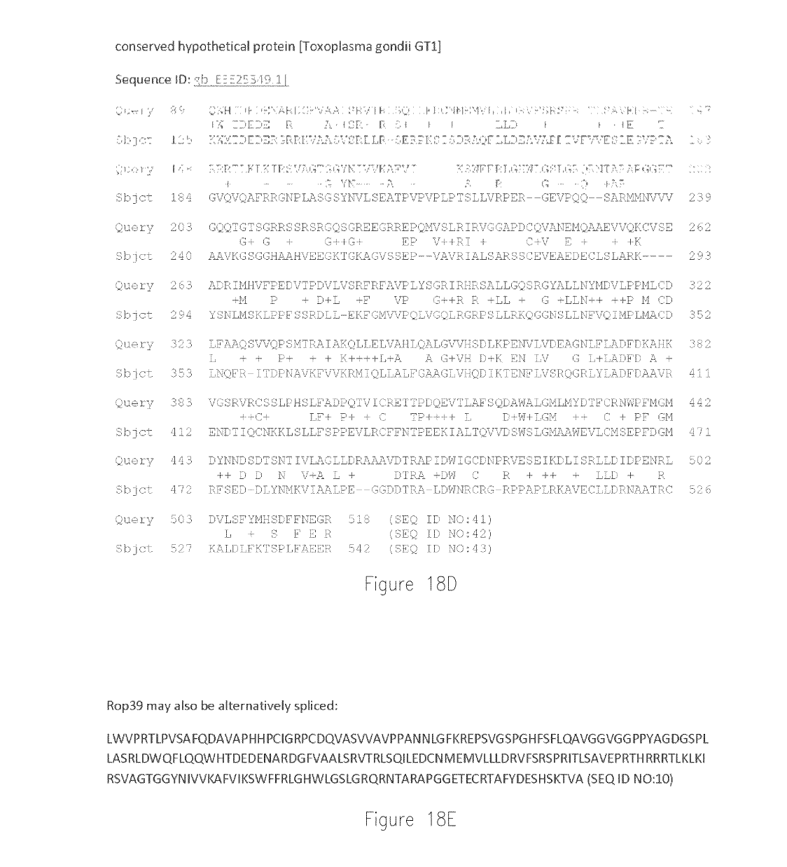

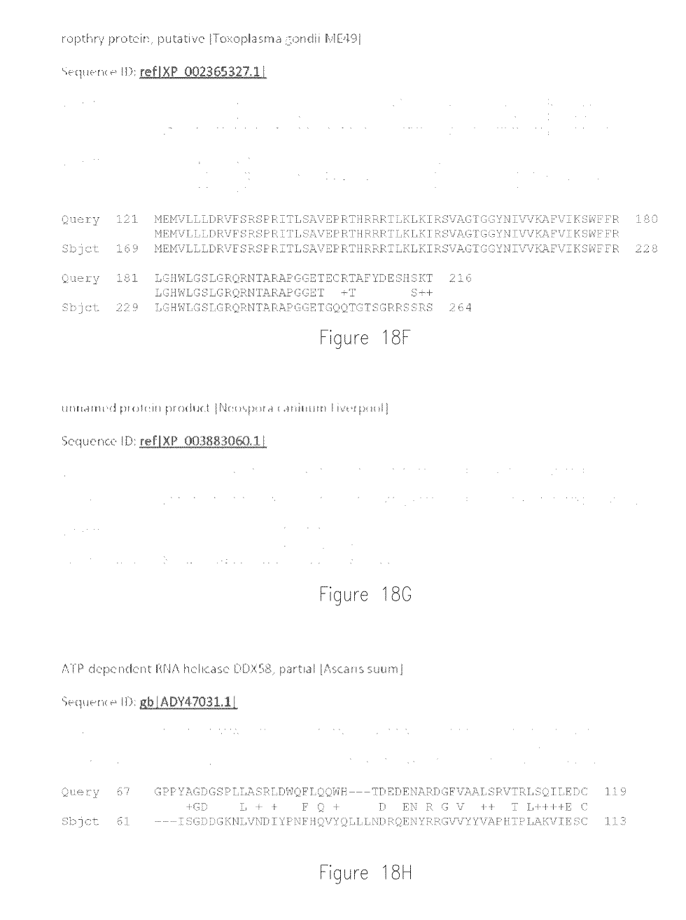

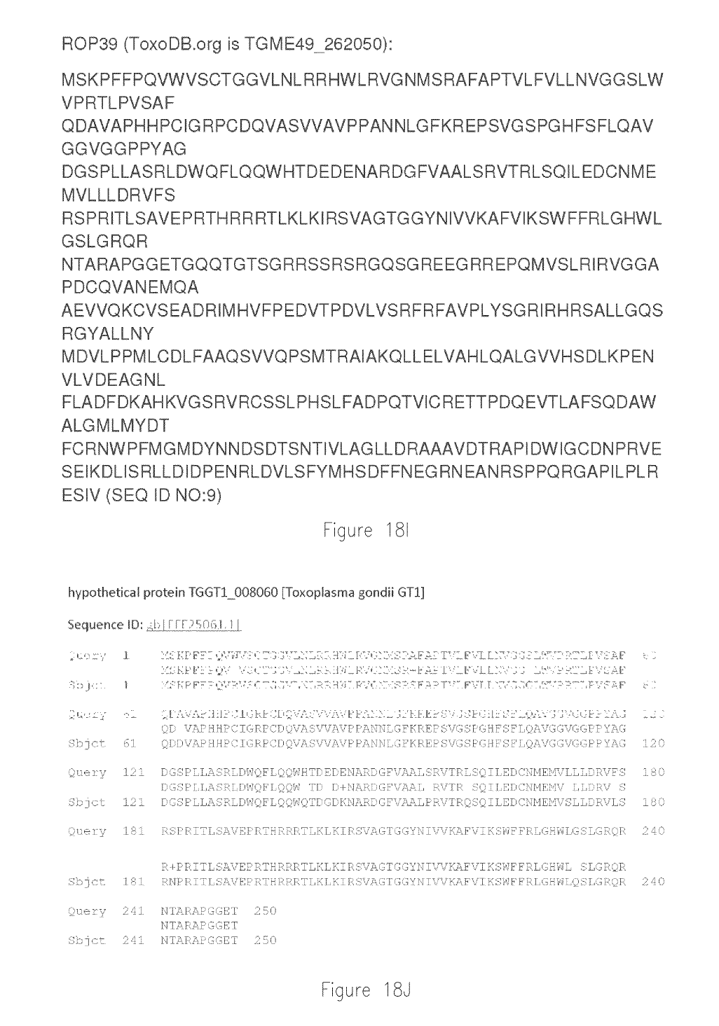

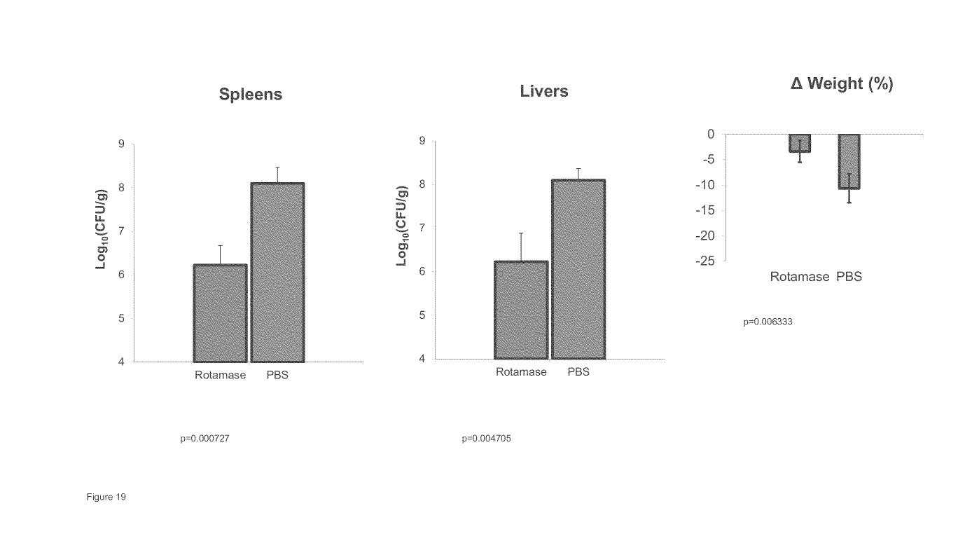

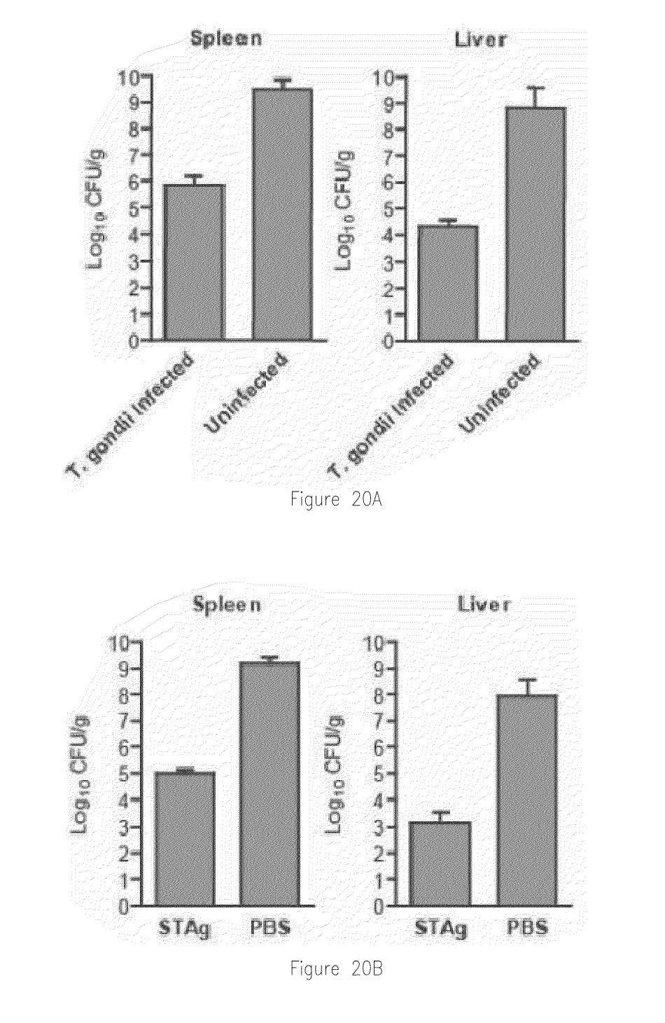

In one embodiment, a protein for use in the compositions and methods of the invention has up to 5% of the residues, e.g., 1, 2, 3, 4, 5, 6, 7, 8, 9, 10 or 11 residues, in SEQ ID NO:4, SEQ ID NO:9 or SEQ ID NO:10 in FIG. 18 substituted with conservative or nonconservative substitutions, up to 10% of the residues in SEQ ID NO:4, SEQ ID NO:9 or SEQ ID NO:10 in FIG. 18 substituted with conservative substitutions, or up to 20% of the residues in SEQ ID NO:4, SEQ ID NO:9 or SEQ ID NO:10 in FIG. 18 substituted, or any combination thereof. In one embodiment, the substitutions include those at one or more positions marked with bold underlined residues in FIG. 18. In one embodiment, a protein for use in the compositions and methods of the invention has up to 5% of the residues in SEQ ID NO:4, SEQ ID NO:9 or SEQ ID NO:10 in FIG. 18 substituted with conservative substitutions, and up to 5% of the residues in SEQ ID NO:4, SEQ ID NO:9 or SEQ ID NO:10 in FIG. 18 substituted with non-conservative substitutions, or up to 10% of the residues in SEQ ID NO:4, SEQ ID NO:9 or SEQ ID NO:10 in FIG. 18 substituted with conservative substitutions, and up to 10% of the residues in FIG. 18 substituted with nonconservative substitutions, or any combination thereof, e.g., where the substitutions are at one or more positions marked with bold underlined residues in FIG. 18. Whether a particular amino acid substitution results in a functional polypeptide can readily be determined by assaying the biological activity of the variant polypeptide by methods well known to the art.

### Exemplary Compositions and Methods of the Invention

The urgent need for new treatments against global infectious diseases has brought about novel approaches. For a small number of infectious diseases, such as HIV, combinations of agents create a genetic barrier to resistance that is large enough such that minimal microbial evolution occurs. Nevertheless this strategy has not yet been applied to the treatment of most infectious diseases. Augmenting the immune arm in combination with traditional antimicrobials offers the possibility of treating infectious diseases without pressuring microbes down a specific evolutionary path that leads to resistance.

The present disclosure generally relates to immunomodulatory compositions and methods for treating, inhibiting or preventing microbial infections in mammals. Active antimicrobial proteins from STAg may be purified and/or heterologously expressed in cells and isolated. In particular, the present invention relates to immunomodulatory compositions and methods which employ one or more isolated proteins, such as isolated native or recombinant profilin or Rop 39 from Toxoplasma or cyclophilin, or related proteins as described herein, recombinant virus or cells, including live attenuated or avirulent recombinant Toxoplasma, e.g., Toxoplasma gondii, including tachyzoites, bradyzoites and/or oocysts, inactivated recombinant Toxoplasma, e.g., inactivated via chemical or heat treatment, or soluble extracts thereof, which cells express one or more of the immunomodulatory proteins of the invention and optionally may express one or more other gene products. Methods for preparing and isolating recombinantly expressed proteins, e.g., proteins expressed as a His tag fusion in E. coli, are known to the art.

T. gondii is an intracellular parasite which is classified among the Coccidia. This parasite has relatively broad host range, infecting both mammals and birds. T. gondii is ubiquitous in nature and during the asexual cycle, occurring in any warm blooded animal, it exists in two forms: the tachyzoite and the bradyzoite. Tachyzoites, found during acute infection, are the invasive form capable of invading all nucleated mammalian cells. After the acute stage of infection, tissue cysts called bradyzoites are formed within host cells and persist within the host organism for the life of the host. Cysts are important in transmission of infection, especially in humans, as the ingestion of raw or undercooked meat can result in the ingestion of bradyzoites which can infect the individual resulting in an acute infection. Oocysts represent the end product of sexual reproduction which occurs only in the intestinal lining of the cat family from which they are excreted in the feces.

The compositions of the invention are effective at preventing, inhibiting or treating microbial infections, e.g., other than T. gondii infections, including viral infections such as influenza infection (including infection with pathogenic avian H5N1 viruses), bacterial infections such as infection with L. monocytogenes, and infections of various parasites such as Plasmodium (e.g., Plasmodium berghei and Plasmodium falciparum), which is known to cause cerebral malaria. In particular, administration of a composition of the invention to a mammal after microbial infection, e.g., up to about 48 hours, about 96 hours, or longer after microbial infection, inhibits the microbial infection, e.g., symptoms thereof, and may also reduce the mortality rate in treated mammals infected with an otherwise lethal dose of microbes. Moreover, inoculation of a mammal with a composition of the invention up to four months prior to exposure to influenza virus may provide protection against influenza infection. In addition, a composition of the invention may protect or lead to an enhanced immune response in a mammal infected with a microbe from later re-infection with the same or a different microbial pathogen.

A protein useful in the compositions and methods of the invention may be identified in and/or isolated from any T. gondii strain including extracts (e.g., STAg) from attenuated or avirulent recombinant T. gondii. The T. gondii may be grown in a susceptible tissue culture and isolated therefrom for use in the compositions or methods, or may be used to prepare a soluble antigen extract thereof. The T. gondii used to prepare the soluble antigen extract-containing compositions, such as STAg preparations, can be obtained from any suitable source. In one embodiment, the T. gondii used to prepare a soluble antigen extract-containing composition is a wild-type T. gondii. In another embodiment, the T. gondii used to prepare a soluble antigen extract-containing composition is a live attenuated or avirulent strain of T. gondii. Suitable strains of attenuated or avirulent T. gondii are known in the art and include, for example, strains derived from PruΔ (a hypoxanthine-xanthine-guanosine phosphoribosyl transferase deletion strain), such as N28E2, 73F9, as well as others described in Frankel et al. (2007), the disclosure of which is incorporated by reference herein (see Table 1). Table 1 lists 39 mutants with about 10-fold reduction in the number of cysts per brain compared with infections with wild-type parasites. The second column contains the percentage of cysts per brain for each mutant compared with wild-type (% WT), along with the number of mice infected with each mutant in parenthesis. For the 24F9 mutant, the mice infected with wild-type parasites died during acute infection for that experiment, thus, the number of cysts per brain for wild-type was estimated at 10,000 (typical for wild-type infections). The attenuated strain 73F9 is a Type II strain that was deposited on behalf of Wisconsin Alumni Research Foundation with the American Type Culture Collection (ATCC), 10801 University Blvd, Manassas, Va. 20110-2209, on Jun. 11, 2009. The accession number for 73F9 is PTA-10117. The mutant strain of T. gondii N28E2 is a Type II strain that was deposited with the ATCC on Jun. 11, 2009. The accession number for N28E2 is PTA-10118.

TABLE 1

| | % WT (no. | | | |
|---|---|---|---|---|
| Mutant | of mice) | Chromosome | Location | annotation |
| 71C2 | 6(3) | Ia | 411900 | |
| 9xG4 | 7(7) | Ia | 599455 | |
| 101F9 | 5(2) | III | 1202200 | ribosomal protein L15 putative |
| 57G3 | 9(2) | IV | 2003950 | |
| 26F7 | 5(2) | IV | 1407661 | |
| 73F9 | 7(6) | V | 1367207 | regulator of chromosome |
| | | | | condensation related |
| 88E8 | 5(11) | V | 1009640 | |
| 9B5 | 10(2) | VI | 178451 | |
| 19C3 | 4(2) | VI | 178451 | |
| 29C3 | 3(2) | VI | 66771 | family domain-containing |
| | | | | transmembrane protein |
| 98F7 | 4(2) | VI | 2577040 | PX domain-containing protein |
| 12F7 | 7(6) | VIIa | 1175720 | cyclophilin lysyl-tRNA synthetase |
| 38C3 | 3(3) | VIIa | 2463700 | |
| KMME6 | 5(12) | VIIb | 1785558 | |
| 13E10 | 9(2) | VIIb | 3944360 | synaptobrevin-like protein, L-type |
| | | | | amino acid transporter-related |
| 68C6 | 4(4) | VIII | 5874600 | |
| 64F2 | 14(1) | IX | 1352099 | acyl-CoA dehydrogenase, |
| | | | | short/branched chain specific, |
| | | | | mitochondrial (precursor) |
| 90B2 | 33(2) | IX | 1352051 | acyl-CoA dehydrogenase, |
| | | | | short/branched chain specific, |
| | | | | mitochondrial (precursor) |
| 91G8 | 3(2) | IX | 3220845 | |
| PRD4 | 3(12) | IX | 5321038 | patatin-like phospholipase |
| | | | | domain containing protein |
| 49E10 | 7(4) | X | 3362809 | elongation factor G putative |
| 56C2 | 7(2) | X | 1882762 | |
| 44C6 | 5(2) | X | 1666204 | |
| 97F9 | 10(2) | X | 5856020 | ImpB/MucB/SamB family domain- |
| | | | | containing protein |
| 41E2 | 0.2(4) | XI | 2191215 | |
| 40E4 | 6(4) | XII | 5779451 | |
| 95C5 | 8(2) | XII | 5331130 | |
| 42F7 | 11(2) | XII | 109850 | staphylococcal nuclease |
| | | | | homologue domain-containing |
| | | | | protein |
| 24F9 | 8(2) | XII | 4733853 | |
| 39B2 | 6(2) | XII | 78139 | small nuclear ribonucleoprotein |
| | | | | (snRNP) |
| 40F3 | 3(2) | XII | 327168 | |
| 40B2 | 6(2) | XII | 327168 | |
| 18C5 | 6(3) | TGG994746 | 45323 | SPX domain-containing protein |
| 7xC11 | 5(9) | TGG994767 | 15632 | |
| 26G5 | 8(4) | | | |
| 37B11 | 7(3) | | | |
| 87D10 | 1(2) | | | |
| 37B2 | 5(2) | | | |
| 91E4 | 1(2) | | | |

The compositions may be produced by growing T. gondii under artificial conditions, for example in tissue cultures, such as described in the examples of the present disclosure, or in vivo in felines. In one exemplary embodiment, T. gondii strains are propagated in human foreskin fibroblast monolayers or in other susceptible tissue cultures, under standard conditions, e.g., at 37° C. with 5% CO₂. A susceptible tissue culture is intended to include a tissue culture that, when inoculated with T. gondii, is able to grow the parasite tachyzoites. Other non-limiting examples of suitable tissue cultures that may be used to grow T. gondii include any nucleated cell such as Vero cells, Chinese hamster ovary (CHO) cells, RAW 264.7 cells (a mouse macrophage cell line), and the like.

A T. gondii culture may be regularly tested for mycoplasma contamination to ensure it is substantially mycoplasma-free. Testing for mycoplasma contamination may be done using any suitable method. Kits for testing cultures for mycoplasma contamination are also commercially available. Examples of such kits include the MycoAlert® Mycoplasma Detection Kit, available from Lonza, Inc. (Basel, Switzerland).

T. gondii tachyzoites may be formulated into a composition of the invention. The culture of T. gondii may be provided in a purified or an unpurified form. In some embodiments, the T. gondii tachyzoites may be purified or partially purified from the host cells prior to formulating the T. gondii into the composition. This may be achieved by lysing the host cells using any suitable technique, such as syringe lysing.

Protein may be isolated from STAg preparations. STAg-containing preparations may be prepared by any conventionally known technique. In one particular example, STAg is prepared as described in the examples set forth herein. Briefly, T. gondii is grown under standard conditions, as described above. When the parasites begin to lyse the host cells, monolayers are scraped, syringed passed through a needle (e.g., 27-gauge), pelleted via centrifugation (e.g., at 100,000×g), washed (e.g., with PBS), and resuspended. The STAg preparation may then be prepared by sonicating the T. gondii suspension under suitable conditions (e.g., with five 30 second pulses). Other procedures for preparing STAg preparations are known in the art. Advantageously, the STAg-containing preparations are non-infectious. As such, STAg may be prepared from wild-type T. gondii, as well as from recombinant, e.g., avirulent, live T. gondii. Any art-known technique may be used to produce a mutated or genetically modified T. gondii strain that is avirulent, including but not limited to chemical mutagenesis and genetic engineering. Examples of suitable avirulent strains include the above-described 73F9 and N28E2 T. gondii strains.

For compositions comprising inactivated T. gondii, the T. gondii may be inactivated using any art-known technique including, for example, contact with an inactivating agent such as formalin, beta-propiolactone (BPL), heat, binary ethylenimine (BEI), detergents, or subjecting the culture to freeze/thaw. Any suitable process may be used for heat-killing the T. gondii. In one non-limiting example, the T. gondii may be heat killed by subjecting the T. gondii to temperatures of from about 95° C. to about 100° C. for about 5 minutes.

In one embodiment, a composition of the invention comprises one or more isolated proteins, such as T. gondii protein(s), or recombinant virus or cells expressing one or more proteins, including a protein of the invention, in an amount effective to elicit an antiinfective or antimicrobial response. In one embodiment, the composition modulates the host immune system, thereby allowing for increased ability to ward off infective microbes or protection from pathological immune activation. For instance, recombinant protein may be isolated from a suitable expression system, such as bacteria, insect cells or yeast, e.g., E. coli, L. lactis, Pichia or S. cerevisiae or other bacterial, insect or yeast expression systems, or mammalian expression systems such as T-REx™ (Invitrogen), and isolated protein may be obtained from the native organism, e.g., profilin may be isolated from T. gondii or a recombinant non-Tg cell. For example, to prepare isolated recombinant T. gondii proteins, any suitable host cell may be employed, e.g., E. coli or yeast, to express those proteins. Those cellular expression systems may also be employed as delivery systems, e.g., E. coli or L. lactis, expressing a heterologous lipoxygenase, such as one expressed on the cell surface or in a secreted form. A suitable cellular delivery system may be one for oral delivery. The recombinant protein useful in the compositions and methods of the invention may be expressed on the surface of a prokaryotic or eukaryotic cell, or may be secreted by that cell, and may be expressed as a fusion, e.g., for targeting, for instance, the recombinant protein may be fused to an antibody an antibody or a portion of an antibody, e.g., ScFv or Fc such as a mutant Fc that stabilizes the fusion, or a cell-surface molecule specific for a type of cell, for instance a neutrophil, for purification, e.g., a His tag may be fused to the recombinant protein, or the recombinant protein may be fused to a molecule with a distinct function, e.g., an immune response stimulator, such as an adjuvant, an immune response inhibitor, or the recombinant protein may be modified to alter MHC binding determinants, T-cell receptors, B-cell receptors or antigenic epitopes, or may be linked to a molecule that alters solubility (e.g., prevents aggregation) or half-life, e.g., a PEGylated molecule, of the resulting chimeric molecule. In one embodiment, the composition of the invention may comprise a recombinant cell expressing one or more recombinant T. gondii proteins, e.g., on the cell surface or as a secreted protein, or a recombinant T. gondii modified to express one or more heterologous gene products, e.g., proteins expressed by a different pathogen (see, e.g., Charest et al., 2000, which discloses vectors useful to express heterologous gene products in T. gondii).

In one embodiment, the invention provides a method of treating, inhibiting or preventing a bacterial infection, e.g., infection by Listeria or a pan resistant gram-negative bacilli, such as Pseudomonas aeruginosa, or multi-resistant gram-positive bacteria like methicillin resistant Staphylococcus aureus, as well as Mycobacterium tuberculosis, or nontuberculosis Mycobacterium or Nocardia, in a mammal. In one embodiment, the method comprises administering an effective amount of a composition of the invention to the mammal after the mammal has been infected with the bacterium. In one embodiment, the composition comprises isolated profilin, either native or recombinant, from one or more sources. In one embodiment, the method comprises administering an effective amount of a composition of the invention to the mammal before the mammal is infected with the bacterium.

In one embodiment, the present invention is directed to a method of treating, inhibiting or preventing a viral infection in a mammal, e.g., viruses including but not limited to rabies, influenza A, influenza B, influenza C, flaviviruses including West Nile virus and Dengue virus, paramyxoviruses including Respiratory Syncyctial virus, parvoviruses, retroviruses, and gastroenteroviruses including rotavirus, norovirus, and astrovirus. In one embodiment, the method comprises administering an amount of a composition of the invention, e.g., after the mammal has been infected with or exposed to a virus, effective to inhibit or treat the viral infection. In another embodiment, the invention generally relates to vaccines and methods for immunizing a mammal against viral infection. In one embodiment, a composition of the invention is administered to a mammal before the mammal is exposed to the virus. In one embodiment, the pathogen is an influenza virus. In one embodiment, the influenza virus is a H5N1 virus.

In one embodiment, the invention provides a method of treating, inhibiting or preventing a parasite infection in a mammal, e.g., infection by various species of Plasmodium, such as Plasmodium berghei, and Plasmodium falciparum and other Coccidia such as Cryptosporidium parvum, or other protozoan parasites such as Trypanosome brucei, Entamoeba histolytica, Leishmania species and helminth parasites such as Schistosoma mansoni. In one embodiment, the method comprises administering an amount of a composition of the invention to the mammal effective to inhibit or treat the parasitic infection after the mammal has been infected with the parasite. In one embodiment, a composition of the invention is administered to a mammal before the mammal is exposed to the parasite.

In one embodiment, the invention provides a method of treating, inhibiting or preventing a fungal infection in a mammal, e.g., Cryptococcus, Aspergillus, species, Histoplasma capsulatum, Blastomyces dermatitidis, Coccidiomycosis immitis and Penicillium marcenscens. In one embodiment, the method comprises administering an effective amount of a composition of the invention to the mammal after the mammal has been infected with the fungus. In one embodiment, the method comprises administering an effective amount of a composition of the invention to the mammal before the mammal is infected with the fungus.

As will be apparent to one skilled in the art, the optimal concentration of the active agent in a composition of the invention will necessarily depend upon the specific immunomodulatory agent(s) used, the characteristics of the avian or mammal, the type and amount of adjuvant, if any, and the nature of the microbial infection. These factors can be determined by those of skill in the medical and pharmaceutical arts in view of the present disclosure. In general, the active agent(s) in the composition of the invention are administered at a concentration that either modulates antimicrobial activity against microbial infection or modulates an immune response allowing the host to recover from or clear a microbial infection, without significant, harmful or adverse side effects.

Specific dosages may be adjusted depending on conditions of disease, the age, body weight, ethnic background, general health conditions, sex, diet, lifestyle and/or current therapeutic regimen of the mammal, as well as for intended dose intervals, administration routes, excretion rate, and combinations of drugs. Any of the dosage forms described herein containing effective amounts are well within the bounds of routine experimentation and therefore, well within the scope of the instant disclosure.

A composition may comprise a protein of the invention in an amount of from about 100 μg per mL to about 1000 μg per mL, in some instances from about 200 μg per mL to about 1000 μg per mL, and in some instances from about 500 μg per mL to about 1000 μg per mL. In one embodiment, the composition comprises an amount of about 1 μg to about 200 μg of protein per dose for a mammal weighing about 20 to 25 g. In one embodiment, the composition comprises a protein of the invention an amount of about 1 mg to about 1000 mg, e.g., about 10 mg to about 100 mg, or an amount of about 0.1 μg to about 1000 μg, e.g., about 1 μg to about 10 μg. In one embodiment, the composition comprises a protein of the invention an amount of about 20 μg/kg to about 2000 μg/kg, e.g., about 50 μg/kg to about 500 μg/kg or about 100 μg/kg to about 400 μg/kg.

The desired dose of the composition may be presented in a continuous infusion, a single dose, or as divided doses administered at appropriate intervals, for example as two, three, four or more sub-doses per day. Optionally, a dose of composition may be administered on one day, followed by one or more booster doses spaced as desired thereinafter. In one exemplary embodiment, an initial dose is given, followed by a boost of the same composition approximately two to four days later. In one particular embodiment, the mammal is administered a first dose of the composition at about 48 hours post-infection and a second dose of the composition at about 96 hours post-infection. Other dosage schedules may also be used, e.g., prophylactic use during an outbreak or pandemic to decrease morbidity post infection.

Following an initial administration of the composition, mammals may receive one or several booster doses adequately spaced thereafter. In some embodiments, the booster doses comprise the same amounts and type of active agent as the initial administration. In other embodiments, the booster doses may comprise a reduced amount and/or a different type of active agent, for instance, the original inoculum may include isolated (native or recombinant) profilin and the booster may be STAg of a live avirulent T. gondii.

In addition to the recombinant virus, recombinant cells or soluble extracts thereof or isolated protein, or combinations thereof, the composition of the invention may further comprise one or more suitable pharmaceutically acceptable carriers. As used herein, the term “pharmaceutically acceptable carrier” refers to an acceptable vehicle for administering a composition to mammals comprising one or more non-toxic excipients which do not react with or reduce the effectiveness of the pharmacologically active agents contained therein. The proportion and type of pharmaceutically acceptable carrier in the composition may vary, depending on the chosen route of administration. Suitable pharmaceutically acceptable carriers for the compositions of the present disclosure are described in the standard pharmaceutical texts. See, e.g., “Remington's Pharmaceutical Sciences”, 18^{th} Ed., Mack Publishing Company, Easton, Pa. (1990). Specific non-limiting examples of suitable pharmaceutically acceptable carriers include water, saline, dextrose, glycerol, ethanol, or the like and combinations thereof.

Optionally, the composition may further comprise minor amounts of auxiliary substances such as agents that enhance the antimicrobial effectiveness of the preparation, stabilizers, preservatives, and the like.

In one embodiment, the composition may also comprise a bile acid or a derivative thereof, in particular in the form of a salt. These include derivatives of cholic acid and salts thereof, in particular sodium salts of cholic acid or cholic acid derivatives. Examples of bile acids and derivatives thereof include cholic acid, deoxycholic acid, chenodeoxycholic acid, lithocholic acid, ursodeoxycholic acid, hydroxycholic acid and derivatives such as glyco-, tauro-, amidopropyl-1-propanesulfonic-, amidopropyl-2-hydroxy-1-propanesulfonic derivatives of the aforementioned bile acids, or N,N-bis (3Dgluconoamidopropyl) deoxycholamide. A particular example is sodium deoxycholate (NaDOC).

Examples of suitable stabilizers include protease inhibitors, sugars such as sucrose and glycerol, encapsulating polymers, chelating agents such as ethylene-diaminetetracetic acid (EDTA), proteins and polypeptides such as gelatin and polyglycine and combinations thereof.

Optionally, the composition may further comprise an adjuvant in addition to the recombinant virus, recombinant cells or soluble extracts thereof or isolated protein described herein. Suitable adjuvants for inclusion in the compositions of the present disclosure include those that are well known in the art, such as complete Freund's adjuvant (CFA) that is not used in humans, incomplete Freund's adjuvant (IFA), squalene, squalane, alum, and various oils, all of which are well known in the art, and are available commercially from several sources, such as Novartis (e.g., Novartis' MF59 adjuvant).

Depending on the route of administration, the compositions may take the form of a solution, suspension, emulsion, or the like. A composition of the invention can be administered intranasally or through enteral administration, such as orally, or through subcutaneous injection, intra-muscular injection, intravenous injection, intraperitoneal injection, or intra-dermal injection to a mammal, e.g., humans, horses, other mammals, etc. Compositions may be formulated for a particular route of delivery, e.g., formulated for oral delivery.

For parenteral administration, the composition of the invention may be administered by intravenous, subcutaneous, intramuscular, intraperitoneal, or intradermal injection, and may further comprise pharmaceutically accepted carriers. For administration by injection, the composition may be in a solution in a sterile aqueous vehicle which may also contain other solutes such as buffers or preservatives as well as sufficient quantities of pharmaceutically acceptable salts or of glucose to make the solution isotonic.

The composition may be delivered to the respiratory system, for example to the nose, sinus cavities, sinus membranes or lungs, in any suitable manner, such as by inhalation via the mouth or intranasally. The composition may be dispensed as a powdered or liquid nasal spray, suspension, nose drops, a gel or ointment, through a tube or catheter, by syringe, by packtail, by pledget, or by submucosal infusion. The composition may be conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the composition and a suitable powder base such as lactose or starch. Examples of intranasal formulations and methods of administration can be found in PCT publications WO 01/41782, WO 00/33813, and U.S. Pat. Nos. 6,180,603; 6,313,093; and 5,624,898, all of which are incorporated herein by reference and for all purposes. A propellant for an aerosol formulation may include compressed air, nitrogen, carbon dioxide, or a hydrocarbon based low boiling solvent. The composition of the invention may be conveniently delivered in the form of an aerosol spray presentation from a nebulizer or the like. In some aspects, the active ingredients are suitably micronized so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns. In one embodiment, the composition is packaged into a device that can deliver a predetermined, and generally effective, amount of the composition via inhalation, for example a nasal spray or inhaler.

### Vaccines

Without wishing to be bound to any particular theory, it is believed that inoculation or immunization of a mammal with a composition of the invention prior to exposure to a microbe may boost the immune response, which in turn enhances protection against subsequent microbial infection. In one embodiment, the innate immune response may increase the amount of neutrophils a and Ly6Chi monocytes present in the mammal, which may lower microbe titers, and/or may increase adaptive immune responses such as heterologous antigen-specific CD8⁺ T cell proliferation, as compared to mammals not pre-inoculated. These T cells proliferate upon exposure to an immunogen, thus providing host immune memory against the microbe. Other mechanisms of protection may also be involved, e.g., the B cell response may be enhanced.

Thus, a composition of the invention may further contain a microbial immunogen that is capable of eliciting an adaptive immune response against the microbe. In one embodiment, the microbial immunogen may be conjugated chemically or recombinantly to a protein from any source. For example, a microbial immunogen may be expressed as a fusion with a profilin and the resulting fusion protein may alter both innate and adaptive immune responses. Therefore, the compositions of the invention may be employed as a vaccine when employed with one or more immunogens of a microbe. Advantageously, those vaccines may prevent infection and/or limit the severity of the infection, or otherwise enhance the adaptive immune response relative to a vaccine that does not include the recombinant virus, recombinant cells or soluble extract thereof or isolated protein in the composition of the invention. In one embodiment, a protein of the invention is employed as an adjuvant for a vaccine, e.g., to induce or enhance the T cell response. In one embodiment, the immunogen is a protein, e.g., a recombinant protein, peptide or polysaccharide, glycoprotein or lipopolysaccharide. In another embodiment, the immunogen may be DNA molecules (polynucleotides) which produce the antigen in cells after introduction of the DNA molecule to the cells (e.g., by transfection). The immunogen may be, for example, a live attenuated microbial pathogen, one or more proteins of the microbe, or a combination thereof. For example, the immunogen may be split virus antigens, subunit antigens (either recombinantly expressed or prepared from whole virus), and/or inactivated whole virus which may be chemically inactivated by any suitable means including, for example, by treating with formaldehyde, formalin, β-propiolactone, or otherwise inactivated such as by ultraviolet or heat inactivation. The immunogen may be provided in a purified or an unpurified form.

The vaccines of the present disclosure may further comprise one or more suitable pharmaceutically acceptable carriers. As used herein, the term “pharmaceutically acceptable carrier” refers to an acceptable vehicle for administering a vaccine to mammals comprising one or more non-toxic excipients which do not react with or reduce the effectiveness of the pharmacologically active agents contained therein. The proportion and type of pharmaceutically acceptable carrier in the vaccine may vary, depending on the chosen route of administration. Suitable pharmaceutically acceptable carriers for the vaccines of the present disclosure are described in the standard pharmaceutical texts. See, e.g., “Remington's Pharmaceutical Sciences”, 18^{th} Ed., Mack Publishing Company, Easton, Pa. (1990). Specific non-limiting examples of suitable pharmaceutically acceptable carriers include saline (e.g., PBS), dextrose, glycerol, or the like and combinations thereof.

In addition, if desired, the vaccine can further contain minor amounts of auxiliary substances such as agents that enhance the antiviral effectiveness of the composition, stabilizers, preservatives, and the like.

Depending on the route of administration, the vaccine may take the form of a solution, suspension, emulsion, or the like. A vaccine of the present disclosure can be administered orally, intranasally, or through parenteral administration, such as through sub-cutaneous injection, intra-muscular injection, intravenous injection, intraperitoneal injection, or intra-dermal injection to a mammal, e.g., humans, horses, other mammals, etc. Typically, the vaccine is administered through intramuscular or intradermal injection.

For parenteral administration, the vaccines of the present disclosure may be administered by intravenous, subcutaneous, intramuscular, intraperitoneal, or intradermal injection, which optionally may further comprise pharmaceutically accepted carriers. For administration by injection, the vaccine may be a solution in a sterile aqueous vehicle which may also contain other solutes such as buffers or preservatives as well as sufficient quantities of pharmaceutically acceptable salts or of glucose to make the solution isotonic.

The vaccine may be delivered locally to the respiratory system, for example to the nose, sinus cavities, sinus membranes or lungs, in any suitable manner, such as by inhalation via the mouth or intranasally. The vaccines can be dispensed as a powdered or liquid nasal spray, suspension, nose drops, a gel or ointment, through a tube or catheter, by syringe, by packtail, by pledget, or by submucosal infusion. The vaccines may be conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the vaccine and a suitable powder base such as lactose or starch. Examples of intranasal formulations and methods of administration can be found in PCT publications WO 01/41782, WO 00/33813, and U.S. Pat. Nos. 6,180,603; 6,313,093; and 5,624,898, all of which are incorporated herein by reference and for all purposes. A propellant for an aerosol formulation may include compressed air, nitrogen, carbon dioxide, or a hydrocarbon based low boiling solvent. The vaccines of the present disclosure can be conveniently delivered in the form of an aerosol spray presentation from a nebulizer or the like. In some aspects, the active ingredients are suitably micronized so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns. In one embodiment, the vaccine is packaged into a device that can deliver a predetermined, and generally effective, amount of the vaccine via inhalation, for example a nasal spray or inhaler.

The vaccines of the present disclosure are administered prophylactically. For instance, administration of the vaccine may be commenced before or at the time of infection. In particular, the vaccines may be administered up to about 1 month or more, or more particularly up to about 4 months or more before the mammal is exposed to the microbe. Optionally, the vaccines may be administered as soon as 1 week before infection, or more particularly 1 to 5 days before infection.

The desired vaccine dose may be presented in a single dose or as divided doses administered at appropriate intervals, for example as two, three, four or more sub-doses per day. Optionally, a dose of vaccine may be administered on one day, followed by one or more booster doses spaced as desired thereinafter. In one exemplary embodiment, an initial vaccination is given, followed by a boost of the same vaccine approximately one week to 15 days later.

### Pharmaceutical Formulations

The compositions of this invention may be formulated with conventional carriers and excipients, which will be selected in accord with ordinary practice. Aqueous formulations are prepared in sterile form, and when intended for delivery by other than oral administration, will generally be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone they may be present as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

Pharmaceutical formulations according to the present invention may include one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for intrapulmonary or nasal administration may have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of a given condition.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Exemplary unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

An effective dose of an active ingredient depends at least on the nature of the condition being treated, toxicity, whether the active ingredient is being used prophylactically (e.g., lower doses may be employed), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day, including from about 0.01 to about 5 mg/kg body weight per day, or from about 0.05 to about 0.5 mg/kg body weight per day. For example, the daily candidate dose for an adult human of approximately 70 kg body weight will range from 1 mg to 1000 mg, e.g., from 5 mg to 500 mg, and may take the form of single or multiple doses. For instance, about 5 mg to about 750 mg, e.g., about 10 mg to about 70 mg, or any integer in between, of the active ingredient may be administered to a human (see Reagsn-Shaw et al., 2008).

The invention will be described by the following nonlimiting examples.

### Example I

### T. gondii Gene Products that Stimulate Innate Immunity in a TLR11-Independent Manner

### Materials and Methods

### Mice.

All mice used in this study were on a C57BL/6 background and used at 6-8 weeks of age. Wild-type (WT) C57BL/6 mice were purchased from National Cancer Institute (Harlan, Frederick, Md.). TLR11^{-/-} mice were a kind gift from F. Yarovinsky (Yarovinsky et al., 2005). All animals were housed and bred under specific pathogen free conditions at an AALAC accredited facility at the University of Wisconsin School of Medicine and Public Health. All experiments were conducted in accordance with IACUC approved protocol M01545.

### TgPRF

A purified recombinant His-tagged T. gondii profilin (rPRF) was a kind gift from F. Yarovinsky or was purified similar to methods described in Yarovinsky et al. (2005). WT mice were stimulated with rPRF (0.1 μg) in 200 μL PBS via retro-orbital i.v. injection. TLR11^{-/-} mice were stimulated with rPRF (4 μg). Control mice were mock-stimulated with PBS. For L. monocytogenes experiments, mice received a single rPRF-stimulation either at 24 hours pre-, 4 hours pre- or 4 hours post-bacterial infection. For flow cytometry experiments, mice received a single rPRF-stimulation and spleens and blood were collected 4 or 24 hours later.

T. gondii Infections

Tachyzoite PruΔ T. gondii parasites were routinely passaged in tissue culture as described in Van et al. (2007). Generally, propagation of the T. gondii tachyzoites was done in human foreskin fibroblast (HFF) monolayers at 37° C. with 5% CO₂ under standard conditions (Ware & Kasper, 1987) and tested to be mycoplasma-free (Lonza, Switzerland). All attenuated parasites were derived from PruΔ (hypoxanthine-xanthine-guanosine phosphoribosyl transferase deletion) strain. Mice were infected with up to 2500 tachyzoites i.p and fed Uniprim diet (TD.06596, Harlan Tekland, Indianapolis, Ind.) on days 7-14 to prevent death during the acute stage of parasite infection, then a normal diet was resumed from day 15. Animals were allowed to progress at least 30 days, into the chronic stage of T. gondii infection, prior to use in L. monocytogenes experiments. Age matched uninfected control mice were maintained on the same diets as T. gondii infected mice at all times. Brains from infected animals were collected at the time of sacrifice and stained to confirm the presence of T. gondii cysts as described in Mordue et al. (2007).

Listeria monocytogenes Infections

L. monocytogenes strain EGD (Czuprynski et al., 1994) was a kind gift from C. Czuprynski (Czuprynski et al., 1994). Frozen stocks were made from an overnight culture grown in BHI medium (BD BBL, Franklin Lakes, N.J.), and an aliquot was streaked onto a fresh blood agar (TSA with 5% sheep blood) plate (BD BBL) for each experiment. On the day of infection, colonies were inoculated into BHI medium and grown to mid-log phase and then pelleted by centrifugation. Bacteria were washed once with PBS then diluted to an appropriate concentration to cause lethal infection (as indicated below). Mice were anesthetized with an isofluorane vaporizer connected to an IVIS 200 imaging system then infected with bacteria in 200 μL via retro-orbital i.v. injection. Animals were monitored daily for clinical signs of disease (ruffled fur, hunched posture, paralysis, etc.) and were euthanized if moribund. Weight loss and bacterial burdens (CFU/g) in the spleen and liver were determined at three days post infection. To determine bacterial burdens, mice were euthanized by CO₂ asphyxiation and the spleen and a section of liver were weighed then homogenized in PBS. Serial dilutions of the homogenates were plated on blood agar plates and CFUs were counted after 48 hours.

WT mice were infected with 6×10⁴ CFU which consistently resulted in death of 100% of control animals. TLR11^{-/-} mice were infected with 4×10⁴ CFU per animal. The inoculum in each infection experiment was confirmed by plating.

### Flow Cytometry

Spleens were dissociated by mechanical disruption, digested with collagenase/dispase (1 mg/ml, Roche, Indianapolis, Ind.) and DNAse I (10 mg/mL, Roche) for 30 minutes at 37° C. then passed through a 70 μm nylon mesh cell strainer (BD). Heparinized blood was collected via cardiac puncture and red blood cells were removed by dextran sedimentation. Remaining red blood cells were lysed with ammonium chloride. Cells were counted and stained at 4° C. in PBS with 0.5 mM EDTA, 0.2% BSA, 0.09% azide, and 2% normal rat serum (Jackson ImmunoResearch, West Grove, Pa.). CD11b-Alexa Fluor 700 and Gr-1-PE, CD11b-PerCpCy5.5, Ly6C-PE, and Ly6GAlexa 700 antibodies were purchased from eBioscience (San Diego, Calif.). CompBeads (BD Biosciences) stained with experimental antibodies were used to set compensation. Data were collected on an LSRII cytometer (BD Biosciences) and analyzed with FlowJo 7.6.1 (TreeStar, Ashland, Oreg.).

### Results

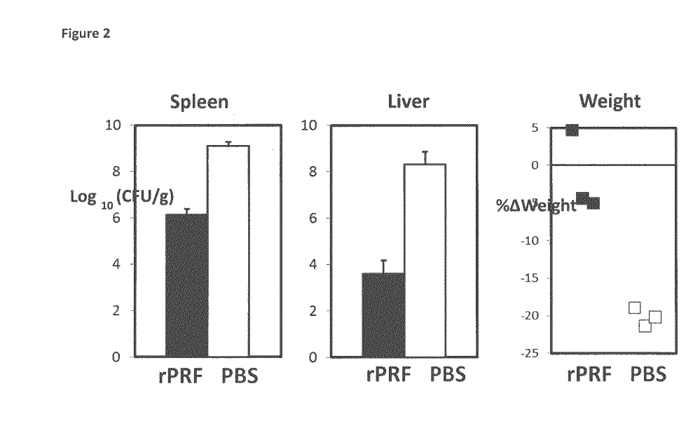

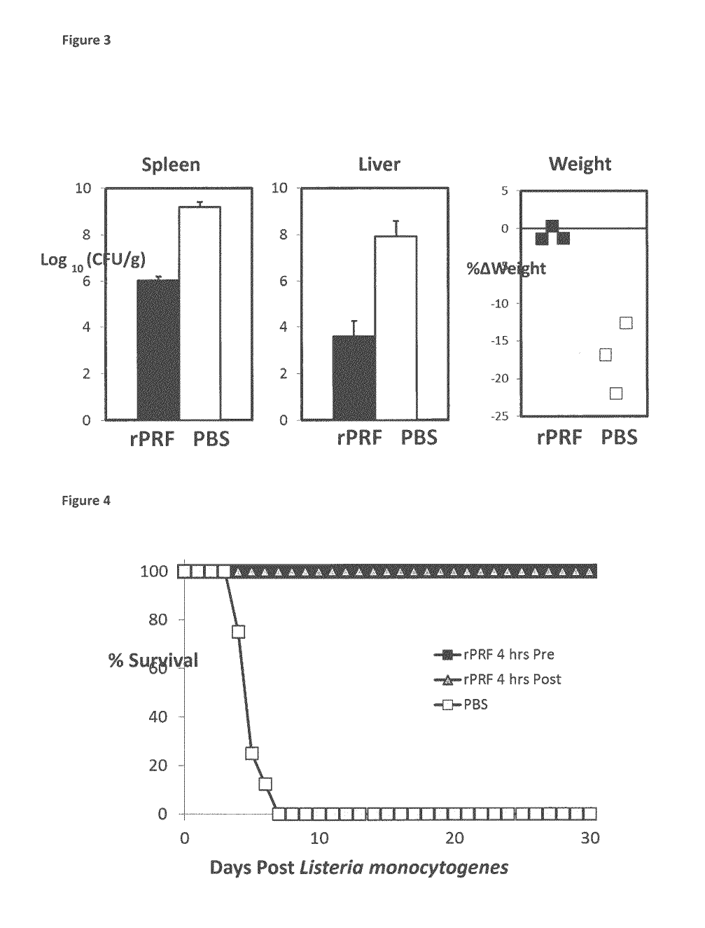

Female C57BL/6 mice (6-8 weeks of age) were treated with rPRF (0.1 μg i.v. in 200 μL PBS) or PBS (i.v. 200 μL) and starting weights recorded. 4 hours later, mice were infected with Listeria monocytogenes EGD (about 6×10⁴ CFU i.v.). Three days later weights were recorded, and spleens and liver were collected, homogenized in PBS and plated in serial dilutions to determine bacterial burden in each organ (FIG. 2). In another experiment, rPRF or PBS was given 24 hours prior to Listeria monocytogenes infection (FIG. 3). FIG. 4 shows percent survival in animals pre-treated with rPRF (4 hours before) or post-treated with rPRF (4 hours after) infection.

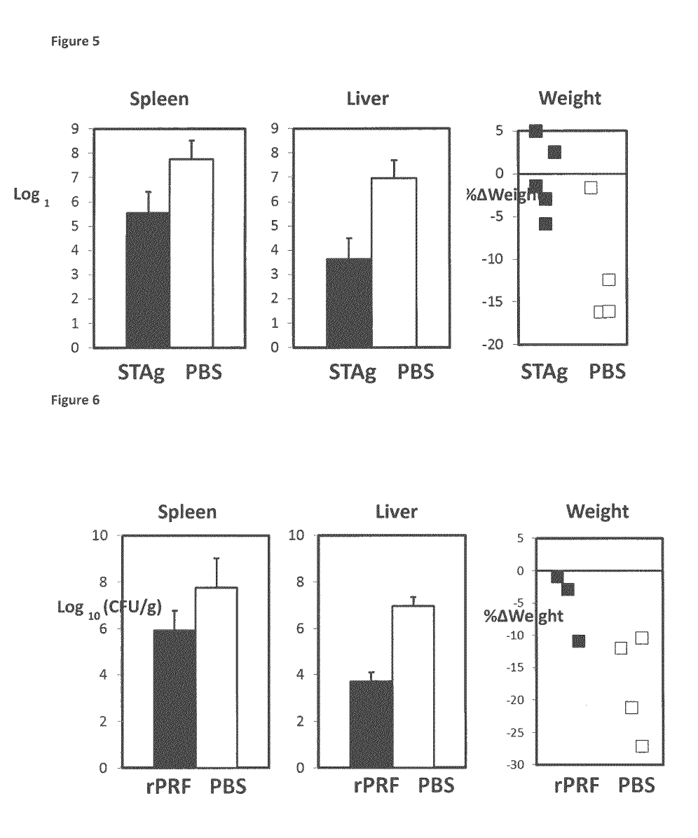

Nearly the same protocol as for wild type mice was used for TLR11^{-/-} mice. Female TLR11^{-/-} mice (6-8 weeks of age) were treated with rPRF (4.0 μg i.v. in 200 μL PBS), or STAg or PBS (i.v. 200 μL) and starting weights recorded. 4 hours later mice were infected with Listeria monocytogenes EGD (about 4×10⁴ CFU i.v. in 200 μL PBS). Three days later weights were recorded, and spleens and liver were collected, homogenized in PBS and plated in serial dilutions to determine bacterial burden in each organ (FIGS. 5-6).

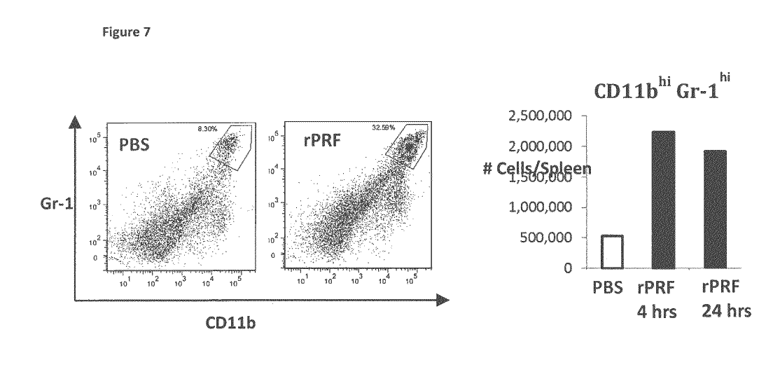

To determine splenic neutrophil numbers, spleens from WT animals were collected 4 or 24 hours after treatment (PRF or PBS), and cells stained with anti-mouse CD11b PerCpCy5.5 and anti-mouse Gr-1 PE antibodies to identify neutrophils (CD11b hi Gr-1 hi population) by flow cytometry. The number of neutrophils as a percent of spleenocytes and the total number per spleen were calculated (FIG. 7).

### Example II

### Use of T. gondii Preparations to Inhibit or Treat Influenza

### Methods

Laboratory Facilities.

All experiments using H1N1 viruses, including work with animals, are performed in a BSL2 containment laboratory. Investigators may be required to wear appropriate personal respirator equipment (RACAL; Health and Safety Inc., Frederick, Md.). Mice may be housed in HEPA-filtered negative pressure cages (M.I.C.E. racks; Animal Care Systems, Littleton, Colo.).

Viruses, Cells, and Viral Infections.

H1N1 influenza virus is propagated in Madin Darby canine kidney (MDCK) cells as described in Jones et al. (2006). Viral titers are determined by fifty percent tissue culture infectious dose (TCID₅₀) analysis in MDCK cells (as described in Jones et al., 2006). MDCK cells are cultured in modified Eagle's medium (MEM, CellGro, Herndon, Va.) supplemented with 4.5 g of glucose per liter, 2 mM glutamine, and 10% fetal bovine serum (FBS, Harlan, Madison, Wis.) at 37° C., 5% CO₂.

Chronic Infection of T. gondii Pre-Influenza Infection.

7-8 week old C57BL/6J mice (Jackson Laboratory, Bar Harbor, Me.) are intraperitoneally (i.p) inoculated with differing numbers of wild-type (WT) or heat-inactivated WT (HI). After 1 or 4 months, mice are transported into a CDC-APHIS approved BSL-3 enhanced laboratory, lightly anesthetized, and intranasally infected with 10⁴ TCID₅₀ (1 mouse lethal dose₅₀ [MLD₅₀]) H1N1 influenza virus. Mice are monitored daily for weight loss and clinical signs of infection (Morton et al., 2000). At 0, 3 and 7 days post influenza infection (dpi), sera and tissues are collected from 2 controls and 3 infected mice from each group and monitored for viral titers (Jones et al., 2006), cytokine levels by ELISA (Tumpey et al., 2000) and flow cytometry (Tumpey et al., 2000).

Treatments of Influenza Infected Mice.

Four to 6 week old C57BL/6J or B6.129S7-Rag1^{tm1Mom}/J (Jackson Laboratory, Bar Harbor, Me.) mice are infected with influenza virus as described above and at 48 hours post infection (hpi) (1 treatment) and 96 hpi (2 treatments) mice were intravenously (i.v.) administered 200 μL per mouse PBS, HFF preparation (negative control), or rPRF and monitored as described above.

Flow Cytometry.

Flow cytometry on lung homogenates is performed as described in Tumpey et al. (2000). Briefly, lungs are dissected, lightly minced, and washed in cold PBS. Pooled lungs from each experimental group are placed into RPMI-1640 (Mediatech, Herndon, Va.) supplemented with 2 mg/mL collagenase B (Roche) and single cell populations generated by pushing homogenates through 70 μm cell strainers (BD Falcon). After centrifugation, the cell pellet is resuspended in red blood cell (RBC) lysis solution, washed, cell number quantitated, and 1.0×10⁶ cells per group stained and blocked with 10% normal rat serum (eBioscience) for 30 minutes at 4° C. Cells are then stained with LIVE/DEAD® fixable dead cell staining (Invitrogen) followed by specific staining for different cell populations. 10,000 total live cell events are gated per sample on a BD LSRII flow cytometer. Percent positive are compared to total gated events by FlowJo® Flow Cytometry Analysis Software.

Histopathologic Analysis.

Tissues are collected and fixed in 10% neutral buffered formalin solution, processed, and paraffin embedded. Histopathologic examination is performed by using hematoxylin- and eosin (H&E)-stained sections.

Statistical Analysis.

Statistical significance of the data is determined by using analysis of variance (ANOVA) or Student's t-test on GraphPad Prism (San Diego, Calif.). Results are representative of at least 3 separate experiments with at least 3 mice per group.

### Results

Mice are infected with a lethal dose of H1N1 influenza virus, then at 2 dpi (1 treatment) and 4 dpi (2 treatments), mice are intravenously administered PBS, a negative control preparation, or profilin and monitored for weight loss and clinical signs of infection and at 0, 3 and 7 days post influenza (dpi), sera and tissues are collected to determine viral titers and cytokine levels by ELISA (Tumpey et al., 2000). If viral titers are decreased within 3 dpi, profilin mediated protection may involve up-regulation of innate immune responses.

If the treated mice are protected against re-infection with a 10× lethal dose of influenza 21 days after the initial infection, profilin-containing preparations may help mice survive to develop a protective adaptive immune response, e.g., the profilin-containing preparations work as an adjuvant by driving an innate immune response that is protective, so that an adaptive immune response can develop and provide long-term protection. Alternatively, the profilin containing preparations may enhance adaptive immunity. If profilin administration induces a general immune response, it is less likely to drive viral resistance than agents targeted specifically at viral proteins.

To examine the mechanism of protection, the lungs are isolated on days 5 and 8 post-influenza infection in treated mice and monitored for cytokine levels by ELISA, e.g., levels of IFN-α, IFN-γ and TGF-β□.

To test whether T cells are involved, RAG^{-/-} mice are infected with influenza virus and treated with profilin 2 days post-infection and monitored for morbidity.

### Example III

### Use of Profilin Preparations to Inhibit or Treat Microbial Infections in Humans

A human is administered, for instance, intravenously or intranasally, an effective amount of profilin (e.g., a Toxoplasma profilin), prior to or after microbial infection including but not limited to influenza virus, Listeria or Plasmodium infection. In one embodiment, a human is administered isolated profilin, e.g., intravenously or intranasally, about 2 to 4 days after a suspected exposure to influenza virus. In one embodiment, a human is administered isolated profilin, e.g., intravenously or intranasally, before, such as about 1 to 4 months before a possible exposure to influenza virus.

### Example IV

### Use of T. gondii Profilin-Related Preparations to Inhibit or Treat Other Microbial Infections

One third of the world's most populous countries are at risk for malaria. Each year 350-500 million cases of malaria occur worldwide, and over one million people die, most of them young children in Sub-Saharan Africa.

Despite the vast literature on the pathogenesis of cerebral malaria (CM), a fatal neurological complication that can arise during Plasmodium falciparum infection (Who, 2000), the mechanisms leading to severe brain pathology and death remain largely unknown. T cells and pro-inflammatory cytokines contribute to the brain inflammation, but how they mediate pathology is unclear. Consequently, no specific treatment for cerebral malaria exists. There are many immunological and parasite events that occur during P. falciparum-induced CM, including sequestration of parasitized erthrocytes, localization of leukocytes and platelets at the blood brain barrier (Medara et al., 2006; Silament et al., 1999; Clark et al., 2003; Grau et al., 2003). Due to the ethical constraints when working with human cases, the exact contribution of these immune and parasite events to the final disease has not been completely elucidated (Rema et al., 2006). Even with current anti-malaria drug treatment, the progression to fatal CM remains high (Krishna, 2012; John et al., 2010). Neurological complications are a problem in children that survive CM (Kihara et al., 2006; Idro et al., 2007), which highlights the need for additional treatments.

The murine experimental cerebral malaria (ECM) model induced by Plasmodium berghei ANKA (PbA) has many similarities to the human CM, which include infected RBC (iRBC) sequestration at the blood brain barrier, vascular leakage, and neurological symptoms (Rema et al., 2006; de Souza et al., 2009; Hunt et al., 2010). In susceptible mouse strains, such as C57BL/6, a high inoculum will paralyze mice usually between six to eight days post infection, and death follows shortly after the onset of paralysis. In addition, the ECM model has led to insights into the intricate role the immune system plays during the induction of ECM. Removal of immune cells or cytokines such as T-cells, NK cells, IFN-γ, TNF-α prevent PbA-induced ECM (Fauconnier et al., 2012; Amani et al., 2010; Hermsen et al., 1997; Sun et al., 2003; Grau et al., 1989; Hanson et al., 2007). Using the ECM model, potential treatments (Hochman et al., 2012; Amante et al., 2010; Buchner et al., 2010) and the role of coinfections have been studied.

Coinfections can lead to altered disease outcomes, both positive and negative when compared to infection of a host with a single pathogen. Highlighting this phenomenon, Toxoplasma gondii infections can prevent disease induced by bacteria, protozoan, and viruses in mice (O'Brien et al., 2011; Ruskin et al., 1968; Mahmoud et al., 1976; Gentry et al., 1971; Remington et al., 1969). In addition, T. gondii can reduce P. yoelii blood stage parasites suggesting a potential protective role during Plasmodium-induced disease (Churest et al., 2000).

### Materials and Methods

Cell Culture, Parasite Strains, and Soluble Tachyzoite Antigen (STAg) Extract Preparation.

T. gondii parasites were maintained as tachyzoites and serial passaged on human forskin fibroblasts (HFF) at 37° C. with 5% CO₂ in Dulbecco's modified Eagle's medium (DMEM) supplemented with 10% heat-inactivated fetal bovine serum (Atlanta Biologicals), 1% penicillin-streptomycin (Invitrogen), and 2 mM L-glutamine (Invitrogen). T. gondii strain PruΔHPT was used for the coinfection and general STAg preparation (Mordue et al., 2007). T. gondii strain TATi-1 expressing myc-tagged Profilin (PRF) using the tetracycline transactivator system was previously described in Plattner et al. (2008).

Plasmodium berghei ANKA (PbA) was used to induce experimental CM in C57BL/6J mice and was kindly provided by Bill Weidanz (University of Wisconsin-Madison). PbA was passed through Balb/C mice, blood was collected by tail vein nick or cardiac puncture. Infected RBC (iRBC) parasitemia was enumerated by counting 500 to 1000 RBC from giemsa stained thin blood smears. The percentage of iRBC to total RBC was determined.

STAg was generated as previously described in Denker et al. (1993), using the parasite strains above but with the following alterations. PruDHPT infected HFF cells were incubated at 37° C. until initial parasite egress. Eggressed parasites were collected and the medium was replaced with Hanks Balanced salt solution (HBSS, Thermo Scientific) supplemented with 1 uM Calcium Ionophore (Sigma). Cells were incubated at 37° C. for 6 minutes and freshly eggressed parasites were collected by centrifugation at 550 xg for 10 minutes and washed with DPBS (128 mM NaCl, 2.7 mM KCl, 8 mM sodium phosphate monobasic, 1 mM potassium phosphate) twice. Parasites were enumerated using a hemacytometer, and suspended at a concentration of 4×10⁸ parasites/mL of DPBS and sonicated. The protein mixture was centrifuged at 100,000 xg for 45 minutes and the soluble fraction was collected, aliquoted, and stored at −80° C. For PRF depleted parasites, Toxoplasma infected HFF were incubated in the presence of 1 ug/ml of anhydrotetracycline (ATET, Argos organics) for 3 days (Plattner et al., 2008). Parasites were eggressed by passing dislodged cells through a 25-gauge needle twice. Parasite enumeration, washing, sonication, and ultracentrifugation was performed as stated for Calcium ionophore eggressed parasites. Protein concentration and the level of Toxoplasma β-tubulin were confirmed by BCA and western analysis. Equivalent parasite numbers were injected in each experiment.

Animals, Animal Infections and Treatments.

Balb/C and C57BL/6J were purchased from NCI (Frederick, Md.). IL-12β2R−/− (B6.129S1-iL12rb2tm1Jm/J) and IL-10−/− (B6.129P2-II10tm1Cgn/J) were purchased from Jackson Laboratory (Bar Harbor, Me.) and bred at the Laboratory Animal Resource facility at the University of Wisconsin-Madison. 6-8 week old age matched mice were challenged with 1×10⁶ iRBC given by intraperitoneal (i.p.) injection. For STAg treatment, equivalent volumes of the prepared STAg extract were give intravenously (i.v.) at the indicated times. Recombinant IL-12 (PeproTech, Rocky Hill, N.J.) was administered i.v. at the indicated amount 1 day after PbA infection.

Assessment of Brain Vascular Permeability.

Mice were administered 0.1 ml of 10 mg/mL Evan's blue dye (MP biomedicals) dissolved in DPBS at 7 days after PbA infection or once the percent iRBC was >8%. After one hour, the mice were sacrificed, perfused with 0.9% NaCl saline and tissues were removed and weighed. Evans Blue was extracted by immersing the tissue in formamide and the amount of evans blue was quantified by measuring absorbance at 610 nm.

Quantitative-PCR (Q-PCR).

To measure the amount of PbA DNA associated with the brain, mice were sacrificed at seven days after infection with PbA and perfused with saline to remove PbA genomic DNA that is present in the circulatory system. Brain tissues were collected, minced and genomic DNA was purified using High Pure PCR template purification kit (Roche) from a portion of the minced brain. Q-PCR was performed using primer probe sets specific to PbA 18S rRNA and mouse genomic DNA sequences as previously described with slight modification to amplify PbA 18S regions (57). Target genes were amplified using Absolute Blue QPCR Mix (Thermo Scientific) using iCycler realtime PCR (Biorad). Ct values and were automatically determined by the software. The relative levels of PbA genomic 18S rRNA were normalized to mouse Albumin levels using the following equation (E_{Murine Albumin}^{Ct})/(E_{PbA18S}^{Ct}), where E=10^{(1/m)}b (Rooney et al., 2011; Payne et al., 2011). The primer probe sets used: PbA 18S primers: 5′-TCAACTACGAGCGTTTTAACTGCAAC-3′ (SEQ ID NO:5), 5′-TTGGAATGATGGGAACTTAAAATCTTCCC-3′(SEQ ID NO:6), probe: 5′-6-FAM™ TGCCAGCAG ZEN CCGCGGTAATTC IBKFQ. Murine Albumin primers; 5′-CAATCCTGAACCGTGTGTGTCT-3′ (SEQ ID NO:7), 5′-TTCATCAACTGTCAGAGCAGAGAAG-3′ (SEQ ID NO:8), probe: 5′-FAM CCAAGTGCT ZEN GTAGTGGATCCCTGGTGG IBKFQ. Probes were labeled with 6-carboxyfluorescein™ (6-FAM) fluorophore and ZEN™ Iowa Black®FQ (IBKFQ) double quencher (Integrated DNA Technologies).

Cell Purification and Flow Cytometry.

Brain mononuclear cells (BMNC) were isolated at previously described in Wilson et al. (2005). Perfused brains were extracted, cut into small pieces and further disrupted by passing through an 18-gauge needle. Brain tissues were digested with 25 μg/mL collagenase/dispase (Roche) and 750 μg/mL DNAseI (Roche) for 45 minutes at 37° C. Cells were washed and passed through a 70 μm cell strainer (Falcon). BMNC were purified over a 30%-60% discontinuous gradient after centrifugation at 1000×g for 25 minutes at room temperature. Cells were collected from the interface, washed and enumerated after trypan blue stain for viability. For flow cytometry, cells were washed with FACS buffer (phosphate buffered saline pH 7.4, 0.2% BSA, 1 mM EDTA) and incubated for 15 minutes with Fc block (0.1 μg/mL CD16/32, ebiosciences) prior to conjugated antibody incubation. After antibody incubation, cells were fixed with 2% formaldehyde for 10 minutes. Cells were stained with CD3 eFluor450, CD4 alexa fluor700, CD8 APC-eFlluor780 (ebioscience) to monitor T cell accumulation at the brain. Events were collected on a BD LSR II flow cytometer (BD, Franklin Lakes, N.J., USA). Compensation and analyses were performed using FlowJo (TreeStar, Ashland, Oreg., USA).

Serum Cytokine Quantification.

Serum was prepared from PbA-infected STAg-treated C57BL/6J at the indicated times. Serum cytokines were quantified using a mouse inflammation cytokine bead array (CBA) kit (BD Biosciences). Events were collected and gated using the BD LSR II flow cytometer and FACsDIVA software (BD Biosciences).

Extract Fractionation, Protein Purification and Protein Analysis.

STAg was prepared as above and desalted and buffer changed into 50 mM Tris pH 7.5 using Amicon Ultra 3 kDa molecular weight cut off filters (Millipore). Protein was separated using HighQ anion exchange chromatograph using a 0-1M NaCl discontinuous gradient. Fractions were pooled and buffer changed to DPBS prior to injection. STAg was separated by sodium dodecyl sulfate-polyacrylamide gel electrophoresis and silver stained as described (REF current protocols) or transferred to Hybond-ECL (GE Healthcare) for western immunoblot analysis. The membrane was incubated with the primary antibody for 1 hour diluted as indicated in parentheses: anti-Profilin (1:2000) (Plattner et al., 2008), or anti-β-tubulin (1:5000) (pollard et al., 2009). The membranes were incubated with donkey anti-rabbit immuoglobulin G conjugated to horseradish peroxidase (1:5000) (Jackson Immunoresearch Labs) and detected using ECL plus chemiluminescence system (GE).

### Results

Chronic T. gondii Infection Decreases PbA-Induced Morbidity and the Accumulation of Infected RBC.

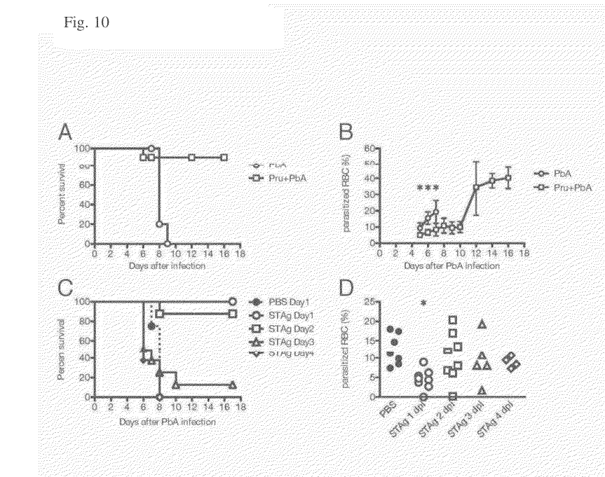

To determine if chronic T. gondii prevents PbA-induced disease, 7-8 week old C57BL/6 mice were infected with 1000 T. gondii parasites. T. gondii-infected mice were challenged with a lethal dose of PbA and monitored daily for CM symptoms. Ninety percent of the T. gondii-infected mice survived when subsequently challenged with PbA whereas zero percent of the mice survived after 9 days after PbA challenge alone (FIG. 10A). Coinfection reduced circulating parasitemia at days five (P<0.025), six (P<0.001), and seven (P<0.025) compared to PbA infection alone (FIG. 10B). The parasitemias remained low in the coinfected animals for the first 9-10 days after PbA infection and then started to increase for the remainder of the monitored time frame even though no CM symptoms were observed. Collectively, these data suggest that T. gondii coinfection prevents the onset of CM.

A non-infectious soluble T. gondii extract (STAg) prevents mortality and reduces the viral load after infection with a high path H5N1 influenza virus in C57BL/6 mice (O'Brien et al., 2011). To determine if STAg was sufficient to prevent a PbA-induced CM, C57B1/6 mice were infected with PbA and IV administered STAg at various times after PbA infection (FIG. 10C). Treatment of PbA infected mice with STAg at one or two days after PbA infection resulted in ≧90% survival. However, mice were not protected when PbA-infected mice were treated with STAg at three or four days which is comparable to PBS treatment alone. STAg treatment reduced the accumulation of iRBC when mice were treated with STAg one day after infection when compared to PBS treatment (P<0.005) (FIG. 10D). However, STAg treatment did not reduce the accumulation of iRBC when treatment occurred on or after two days after infection even though all of the mice were protected from CM induced disease when treated on day two. HFF treatment did not prevent of PbA-infected mice did not prevent CM or reduce iRBC accumulation. Thus, a non-infectious treatment of STAg can prevent PbA-induced CM and can mimic the T. gondii PbA coinfection.

STAg Reduces PbA-Induced Vascular Leakage, Parasite Sequestration and T Cell Localization in the Brain.

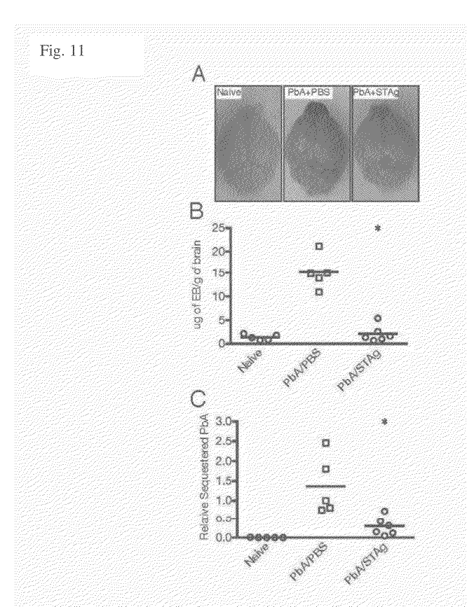

Breakdown of the blood brain barrier (BBB) which leads to vascular leakage in the brains of mice and humans has been associated with CM disease (Amante et al., 2010; Baptista et al., 2010; Thumwood et al., 1988; Medana et al., 2001. To determine if STAg treatment prevented these two hallmarks of PbA-induced CM, Evan's blue dye was used as an indicator of vascular leakage into the tissue to access whether BBB integrity was preserved following STAg treatment. Mice were infected with PbA and treated with STAg two days after infection to allow for similar parasite accumulation and mice were subjected to the assay only once parasitemia was greater than eight percent. PbA-infected mice showed a strong vascular leakage of Evan's blue into the brain tissue. However, STAg treatment reduced the infitration of the dye into the brain tissue and levels of infiltration were similar to naive mice (FIGS. 11A and 11B), suggesting that the blood brain barrier integrity was kept intact.

Accumulation of iRBC and T cells in the brains of infected mice is required to induce CM ((Hermsene t al., 1997; Nitcheu et al., 2003; Amante et al., 2010; Baptista et al., 2010; Grau et al., 1986). To determine if parasites accumulate in the brains of infected mice, the presence of PbA genomes was quantified by q-PCR after intracardiac profusion. The level of parasite genome accumulation decreased by about 4 fold (P<0.01) when STAg treatment was compared to PBS-treated infected mice even though the levels of circulating infected RBC were similar between treated animals when tissues were collected (FIG. 11C). However, STAg treatment did not completely eliminate the accumulation of parasite genomes when we compared STAg treated animals to naïve animals, which suggests that STAg treatment reduces but does not eliminate the accumulation of iRBC in the brains of PbA-infected animals.

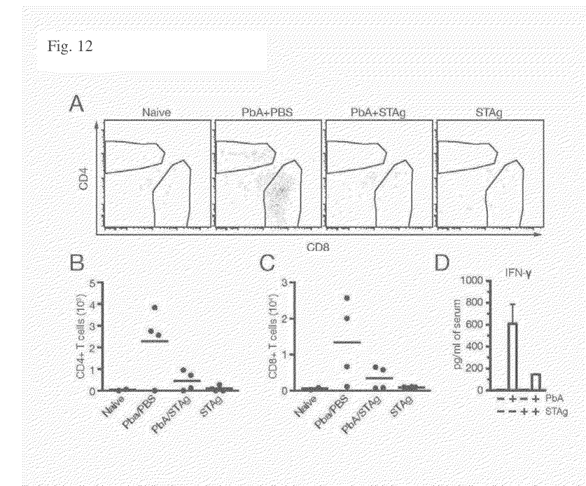

Accumulation of parasites in the brains of PbA infected mice is dependent on the presence of CD8+ and CD4+ T cells in the brain (Amante et al., 2010; Baptista et al., 2010). Due to the reduction of PbA DNA in the brains of infected mice, it was predicted that STAg treatment would reduce or prevent PbA-induced T cell accumulation. To this end, the accumulation of T cells in the brains was monitored by FACS analysis in mice treated with STAg or PBS after intracardiac profusion. PbA infection caused a increase in the number of brain localized CD4+ and CD8+ T cells compared to naive animals (FIG. 12). However, STAg treatment of PbA-infected mice reduced brain localized CD4+ (P<0.06) and CD8+ T (P<0.1) cells compared to PbA infection alone. In addition, the STAg treatment without PbA infection did not stimulate T cell localization to the brain. Collectively, this datum suggests that STAg treatment during PbA is preventing T cell localization, which in turn prevents damage to the brain vasculature.

T cell localization is dependent on IFN-γ accumulation since the depletion of IFN-γ by antibody depletion or the use of IFN-yRKO mice prevented the accumulation of T cells in the brain (Hansen et al., 2007; Belnoue et al., 2008). In addition, TNF-α depletion prevents PbA-induced CM (Grau et al., 1987). Next, the accumulation of cytokines in the serum following PbA infection with or without STAg treatment was investigated. Serum levels of IFN-γ and TNF were measured by cytokine bead array 5 days after PbA infection, which is just prior to the onset of CM symptoms during PbA infection. STAg administration in PbA-infected animals reduced the accumulation of IFN-γ by 3-fold compared to PBS treated PbA-infected animals (FIG. 12D). However, STAg treatment did not reduce the accumulation of TNF-α suggesting that STAg treatment might prevent T cell localization by reducing the accumulation of IFN-γ but not TNF-α (data not shown), which suggests that STAg is only preventing part of the cytokine response that normally occurs during PbA infection. STAg treatment in the absence of PbA infection did not increase any cytokines at a similar time after treatment.

STAg-Induced Protection Requires IL-12.

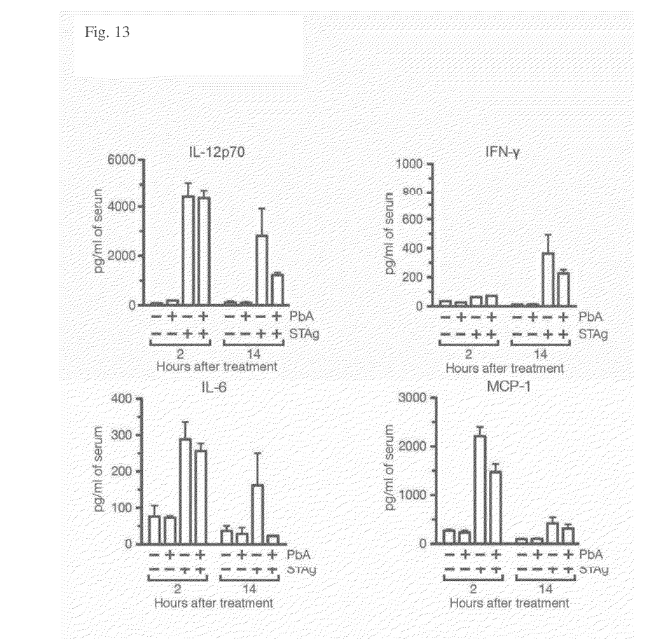

T. gondii induces a strong Th1 response during infection and after STAg administration (Gazzinell et al., 1994; Reis e Sousa et al., 1997). To confirm that STAg treatment was increasing Th1 cytokines early after treatment and treatment during infection that could contribute to STAg-induced protection, serum levels of IFN-γ, IL-12p70, MCP-1, and IL-6 were determined by a cytokine bead array after 2 and 14 hours after STAg treatment by IV administration. STAg treatment with or without PbA infection increased IL-12p70 (>55-fold, p<), MCP-1 (>5-fold, p<), and IL-6 (>3-fold, p<) (FIG. 13) compared to PBS treated mice by two hours after treatment irregardless of infection status. Additionally, STAg treatment increased the accumulation of IFN-γ (>18-fold, p<) compared to PBS treated PbA infected or naïve mice at 14 hours after treatment.

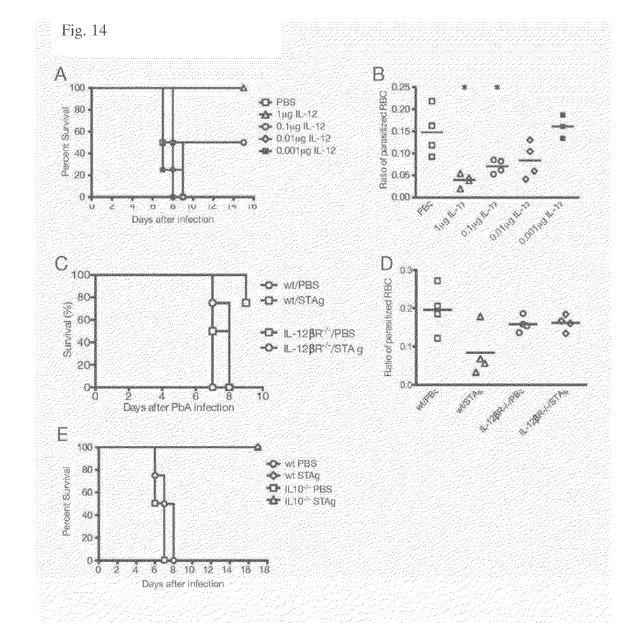

To test whether any of the cytokines that accumulated in the serum were sufficient to prevent PbA-induced CM, increasing amounts of recombinant purified cytokines were injected following PbA infection to see if these cytokines were sufficient to prevent CM symptoms. Infected mice were treated with recombinant IL-12p70 one day after PbA infection and mice were monitored for CM symptoms. Surprisingly, mice that received greater than 0.1 ug of recombinant IL-12p70 had an increase in the level of survival after IL-12 treatment (FIG. 14A). However, treatment of mice with recombinant MCP-1 and IFN-γ did not prevent PbA-induced CM (data not shown). In addition, 1 ug (P<) and 0.1 ug (P<) of IL-12 treatment was able to significantly reduce the accumulation of circulating iRBC compared to PBS treated mice (FIG. 14B), which suggests that IL-12p70 secretion is sufficient to prevent PbA-induced CM. To confirm that IL-12 contributes to STAg induced protection, we infected IL-12βR mice with PbA and monitored mice for CM symptoms after STAg or PBS treatment. STAg treatment did not prevent PbA-induced CM (FIG. 14C) or reduce circulating iRBC (FIG. 14D). Thus, STAg induced protection requires IL-12 and IL-12 expression is sufficient to prevent CM.

T. gondii-induced IL-10 is important during T. gondii infection since the removal of IL-10 during T. gondii infection lead to increased mortality (Suzuki et al., 2000; Gazzinelli et al., 1996). Systemic IL-12 expression induced by T. gondii infection and by IL-12 treatment alone can induce IL-10 expression from NK cells (Perona-Wright et al., 2009). It was hypothesized that STAg induced protection might prevent T cell localization through IL-12 dependent IL-10 expression. To investigate whether STAg induced protection requires IL-10, IL-10−/− mice were infected with PbA and treated mice with STAg one day later. One hundred percent of the mice survived after STAg treatment (FIG. 14D). In contrast all of the wt and IL-10−/− mice treated with PBS succumbed to PbA developed CM symptoms and had to be sacrificed suggesting that IL-10 is not required for STAg induced protection.

T. gondii Profilin is Sufficient to Prevent PbA-Induced CM.

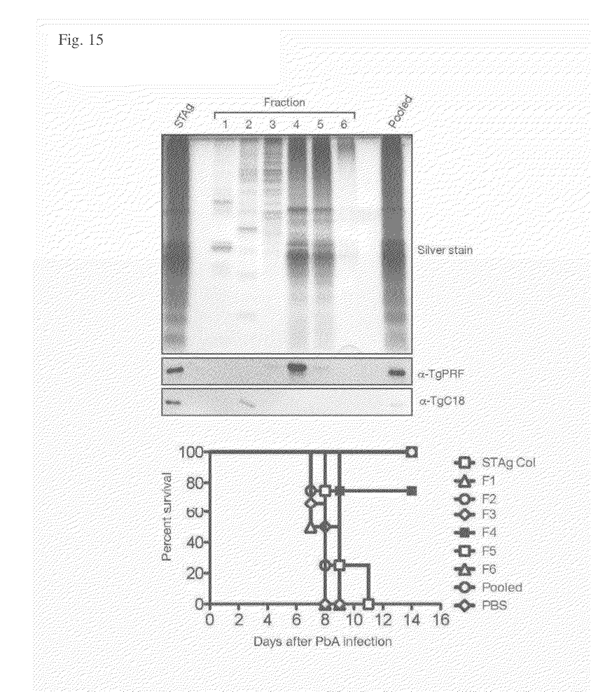

IL-12 is a major product produced during T. gondii infection and STAg treatment. TgProfilin and TgC-18 induce IL-12 expression in spleenic dendritic cells (DC) (Yarovinsky et al., 2005; Aliberti et al. 2003). To identify the active component in STAg, an anion column fractionation approach was used to fractionate STAg. Eluted fractions were pooled, concentrated and C-18 or PRF were detected by western blot analysis. TgC-18 eluted in pooled fraction two and TgPRF eluted in pooled fraction five (FIG. 15A). To determine the protective fraction, fractions were administered to PbA-infected animals one day after infection and CM symptoms and morbidity were recorded. Pooled fraction 5 containing TgPRF induced about 75% protection in wt PbA-infected mice, which was similar to control STAg treated mice (FIG. 15B). Treatment of the PbA-infected mice with the pooled fraction two did not prevent CM, which suggests that TgPRF, but not TgC-18, contributes to STAg-induced protection.

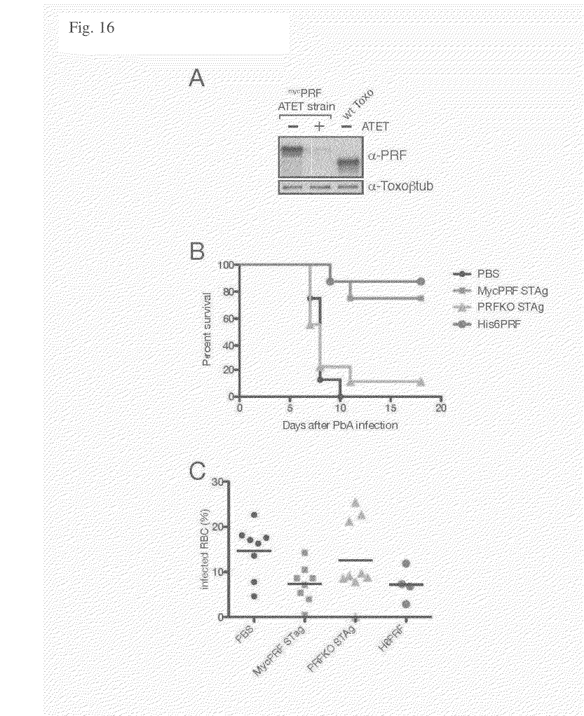

To further demonstrate the contribution of PRF to STAg induced protection, a strain was used that expressed a Myc-tagged TgPRF under the expression of a tetracycline suppressible promoter (Plattner et al., 2008). Use of the suppressible TgPRF T. gondii strain was essential since TgPRF is essential for T. gondii motility. T. gondii tachyzoites were grown in the presence and absence of ATET for four days. After four days, STAg was prepared and PRF levels were determined by western analysis (FIG. 16A). When band intensities were quantified by densitometry, TgPRF was reduced by about 95% compared to untreated T. gondii.

To determine the contribution of TgPRF to STAg's protective activity mycPRF STAg, profilin knock out (PRFKO), or recombinant purified HIS6PRF was administered to PbA-infected animals. STAg preps were administered as close to the minimal dose possible since depletion of TgPRF was not complete (FIG. 16A). Administration of PRFKO STAg and PBS did not prevent PbA-induced mortality and were not significantly different (P>0.05). Importantly, mycSTAg (P<0.01) and purified His6PRF (P<0.01) prevented PbA-induced mortality (FIG. 16B). Purified profilin (P<0.05) and STAg (P<0.05) reduced PbA induced iRBC accumulation compared to PBS. In addition, parasitemia was not significantly different when compared to PBS treated animals (P>0.05). Collectively, these experiments suggest that TgPRF is sufficient to prevent PbA-induced CM.

### Discussion

These studies have shown that Toxoplasma infection, STAg and the Toxoplasma protein TgPRF can lessen the disease induced by Plasmodium species, specifically the induction of cerebral malaria symptoms. The administration of STAg reduced T cell accumulation and delayed iRBC accumulation in the circulatory systems. STAg can only be administered within the first two days after infection prior to the onset of CM symptoms. These studies have shown that TgPRF can prevent the PbA-induced CM symptoms. TgPRF can induce IL-12 production through a TLR11 dependent pathway (Yaronvinsky et al., 2005). The treatment of mice with IL-12 alone was sufficient to prevent experimental CM.

Initially, to determine how STAg prevented PbA-induced CM it was observed that STAg treatment reduced blood brain barrier permeability induced by PbA infection. Consistent with this result, we also observed that STAg treatment reduced the accumulation of PbA genomes in the brain after perfusion suggesting that STAg treatment reduced parasite sequestration. Additionally, STAg treatment reduced CD8+ T cells in the brain as determined by flow cytometry. Perforin and granzyme B produced by CD8+ T cells are a major contributors to blood brain barrier integrity during PbA infection (Nitcheu et al., 2003; Haque et al., 2011). In addition, the reduction of localized T cells also correlated with a reduced IFN-γ response. IFN-γ contributes to the localization of T cells to the brain during PbA infection (Belnoue et al., 2008; Nie et al., 2009; van sden Steen et al., 2008). These experiments suggest that STAg reduced IFN-γ and T cell localization during PbA infection may prevent CM symptoms and mortality.

STAg and specifically TgPRF induce a strong TH1 cytokine response through the TLR11 receptor (Yarovinsky et al., 2005). Knowing that STAg induced IL-12, initially it was attempted to prevent CM symptoms by treating mice with increasing amounts of purified IL-12. Consistent with STAg treatment, IL-12 treatment prevented CM symptoms and delayed the accumulation of iRBC. In addition, STAg treatment of PbA infected IL-12βR−/− mice did not prevent PbA-induced CM. Finally, TgPRF was found to be sufficient to prevent PbA-induced CM mortality. Collectively, these results suggest that STAg induced IL-12 is a protective cytokine that can be induced during STAg or TgPRF-induced prevention of CM.

IL-12 receptor is required for the induction of CM (Faccioner et al., 2012). The IL-12βR−/− mice were still susceptible to PbA-induced CM. The IL-12βR−/− mice were resistant to CM induction when infected after 7 weeks of age but remained susceptible PbA-induced CM if infected around 6 weeks. Why the IL-12βR−/− mice become resistant after 7 weeks of age is unclear but other IL-12 components and receptors can be removed and still produce a CM response (Faccioner et al., 2012). Using this model, the IL-12βR knockout mice were used to show that STAg was not protective in this mouse strain.

How does IL-12 prevent PbA-induced CM? In addition to IL-12 production, it has been observed that the accumulation of IL-10 and IFN-γ production in the serum of STAg treated animals. IL-12 has been shown to induce IL-10 production (Perona-Wright et al., 2009) and IL-10 plays a major role in preventing CM during coinfection of PbA and with non lethal malaria parasites and filarial nematodes ((Niikura et al., 2010; Specht et al., 2010). In addition, IL-10 plays a critical role in limiting inflammation during T. gondii infection (Wilson et al., 2005). Due to the immunosuppressive nature of this cytokine, its role during STAg treatment was investigated by treating IL-10−/− mice with STAg. Both wt and IL-10−/− c57BL/6 mice infected with PbA were protected by STAg treatment and did not develop CM symptoms. Thus, IL-10 was not a major contributor to STAg induced protection.

IFN-γ was observed in response to STAg treatment (FIG. 13). IL-12 induced IFN-γ plays a major role in protecting blood stage parasite replication during Plasmodium chabaudi and other human malaria parasites (Normaznah et al., 1999; Sedegah et al., 1994; Stevenson et al., 1995). STAg and IFN-γ treatment were able to partially prevent influenza disease ((O'Brien et al., 2011). Although it was attempted to prevent CM through using increasing amounts of IFN-γ little success was achieved (data not shown). Due to the major role IFN-γ plays during PbA-induced CM the role of IFN-γ was not further investigated. However, recombinant IL-12 treatment that provided protection during Plasmodium chabaudi or yoelii infections in mice required IFN-γ, TNF-α and nitric oxide production (Sedegah et al., 1994; Stevenson et al., 1995). Thus the source of IFN-γ, the stimulation of the all of the cytokines and additional effectors may be required for protection induced by IL-12 production during PbA-induced CM.

### Example V

Profilin preparations (purified from E. coli') were found to include a few other proteins, one of which, E. coli cyclophilin (peptidyl-prolyl cis-trans isomerase; SlyD), was found to have immune stimulatory properties, e.g., it reduced parasitemia and decreased symptoms in a CM and Listeria monocytogenes model. For example, mice (n=4/group) were treated with 5 μg N-terminal His6 tagged cyclophilin diluted in 200 μL PBS via i.v. injection. 4 hours later the mice were infected with 4×10⁴ CFU Listeria monocytogenes EGD in 200 μL PBS via i.v. injection. Three days later, mice were sacrificed and spleens and sections of the liver weighed and homogenized, then plated to determine CFUs (FIG. 19). Weights were recorded for each mouse at beginning and end of the experiment to calculate weight change over the course of experiment.

Isolated human PBMCs from a healthy donor were stimulated for 4 or 22 hours with cyclophilin (5 μg/mL) or mock stimulated with an equivalent volume of PBS added to the media. RNA was isolated with TRIzol (Invitrogen), treated with DNASe I (Invitrogen), then cDNA was made using Super Script III First Strand Kit (Invitrogen) according to the manufacturer's instructions. Quantification was carried out using gene specific Taqman primer/probe sets (IDT) using Taqman Universal Master Mix (Invitrogen) on an ABI StepOne Plus machine (Invitrogen) and analyzed using the ΔΔCT method, normalized to Gapdh expression and presented as relative expression compared to PBS-stimulated replicates. Cytokine levels in the supernatants were determined by ELISA at the same time points. FIG. 26A shows the fold change in gene expression of human PBMCs stimulated with cyclophilin for 4 or 22 hours. FIG. 26B depicts cytokine levels in supernatants of human PBMCs stimulated with cyclophilin for 4 or 22 hours.

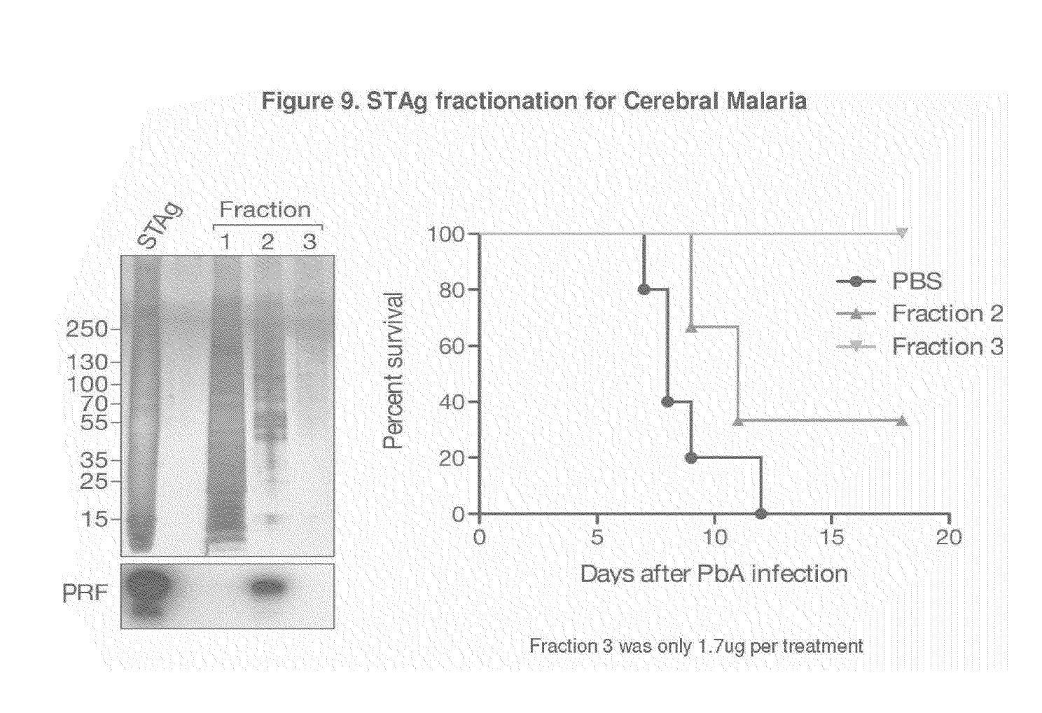

Moreover, fractionation of STAg (see FIG. 9) identified other T. gondii proteins, e.g., Rop39 (Ropthry protein 39), a secreted pseudokinase which may have activity that is not TLR11 dependent. Rop39 DNA was introduced into a bacterial expression construct and used to transform Rosetta E. coli. One colony was inoculated into 10 mL LB+chloramphenicol+selective marker (pET28=kanamycin), and grown overnight at 37° C. with shaking. An overnight culture was diluted 1:20 into fresh media (selection as above) and grown at 37° C. with shaking until the culture reached OD₆₀₀=0.6. Protein expression was induced with IPTG to a final concentration of 1 mM and the culture grown at 37° C. with shaking for 2 hours. Cells were pelleted by centrifuging for 20 minutes at 4000 RPM at RT. The resulting supernatant was removed and resuspended in 1/10 volume lysis buffer (50 mM NaH₂PO₄, 300 mM NaCl, 10 mM imidazole, pH 8.0, protease inhibitors). Lysozyme was added to 1 μg/mL and the mixture was incubated on ice for 30 minutes. The mixture was then sonicated with six 10 second bursts and insoluble material pelleted by centrifuging at 10000 g for 30 minutes at 4° C. The supernatant was transferred to a new tube and washed with 1 mL Ni-NTA beads. The bead containing mixture was shaken at 4° C. for 1 hour, then beads were pelleted by centrifugation at 3500 RPM for 3 minutes at 4° C. The supernatant was removed and the beads washed with 900 μL lysis buffer. The beads were pelleted as above and the supernatant discarded. The washing and pelleting were repeated once. The washed beads were contacted with 500 μL elution buffer (50 mM NaH₂PO₄, 300 mM NaCl, 500 mM imidazole, pH 8.0, protease inhibitors) and the mixture vortexed and centrifuged to pellet the beads. The supernatant was collected and the elution repeated.

### Example VI

### Materials and Methods

Mice.

Unless indicated otherwise, all mice used in this study were on a C57BL/6 background and used at 6-8 weeks of age. Wild-type (WT) mice were purchased from National Cancer Institute (Harlan, Frederick, Md.). IL-12Rβ1^{-/-} (002984, B6.129S1-II2rb1^{tm1Jm}J), IFN-γ^{-/-} (002287, B6.129S7-Ifng^{tm1Ts}/J), Rag1^{-/-} (002216, B6.129S7-Rag1^{tm1Mom}/J), CCR2^{-/-} (004999, B6.129S4-Ccr2^{tm1Ifc}/J) mice were purchased from Jackson Laboratory (Bar Harbor, Me.). All animals were housed and bred under specific pathogen free conditions at an AALAC accredited facility at the University of Wisconsin School of Medicine and Public Health. All experiments were conducted in accordance with IACUC approved protocol. Listeria monocytogenes infections. L. monocytogenes strain EGD was a kind gift from C. Czuprynski. Frozen stocks were made from an overnight culture grown in BHI medium (BD BBL, Franklin Lakes, N.J.), and an aliquot was streaked onto a fresh blood agar (TSA with 5% sheep blood) plate (BD BBL) for each experiment. On the day of infection, colonies were inoculated into BHI medium and grown to mid-log phase and then pelleted by centrifugation. Bacteria were washed once with PBS then diluted to an appropriate concentration to cause lethal infection as indicated for each mouse strain. Mice were anesthetized with an isofluorane vaporizer connected to an IVIS 200 imaging system (Caliper Life Sciences, Hopkington, Mass.) then infected with 200 μL of bacterial suspension via retro-orbital i.v. injection. Animals were monitored daily for clinical signs of disease (ruffled fur, hunched posture, paralysis, etc.) and were euthanized if moribund. Weight loss and bacterial burdens (CFU/g) in the spleen and liver were determined at 72 hour post infection. To determine bacterial burdens, mice were euthanized by CO₂ asphyxiation and the spleen and a section of liver were weighed then homogenized in PBS. Serial dilutions of the homogenates were plated on blood agar plates and CFUs were counted after 48 hours. Wild-type (WT) mice were infected with approximately 6×10⁴ CFU (about 6 LD₅₀'s) which consistently resulted in death or euthanasia of 100% of control animals. IL-12Rβ1^{-/-}, IFN-γ^{-/-}, Rag1^{-/-} NK1.1-depleted, WT RB6-8C5 (Ly6C/Ly6G)-depleted, CCR2^{-/-}, and WT 1A8 (Ly6G)-depleted mice were infected with approximately 1×10⁴CFU, 200 CFU, 8×10⁴CFU, 200 CFU, 8×10³CFU and 8×10³CFU, respectively. These doses chosen were chosen because they resulted in bacterial burdens and weight loss similar to lethally infected WT mice. The inoculum in each infection experiment was confirmed by plating.

Toxoplasma gondii Infection.

Tachyzoites of the Toxoplasma gondii strain PruΔ were routinely passaged in human foreskin fibroblasts as described previously (Mordue and Sibley, 1997). For T. gondii infections, 10 week old WT mice were injected i.p. with 250 tachyzoites. In order to increase the number of animals that survived greater than 30 days into chronic infection, T. gondii-infected and control uninfected mice were all fed a diet containing sulfadiazine (1,365 ppm) and trimethoprim (275 ppm) (TD.06596, Harlan Teklad, Madison, Wis.) from days 9 through 14 post T. gondii infection, then returned to a normal diet on day 15 through the duration of the experiment. To confirm the presence or absence of T. gondii infection in surviving animals, blood was collected via the lateral tail vein on day 28 and assayed for seropositivity against T. gondii antigens by ELISA. Mice were infected with Listeria monocytogenes as described above on day 36 post T. gondii infection and bacterial burdens in the spleen and liver were determined 72 hours later. At the conclusion of the experiment brains from T. gondii-infected mice were homogenized and stained with fluorescein conjugated Dolichos biflorous agglutinin (Vector Laboratories, Burlingame, Calif.) to confirm the presence of bradyzoite cysts as described previously (Mordue et al., 2007).

STAg.

Soluble T. gondii antigens (STAg) were made from sonicated tissue culture grown tachyzoites (4×10⁸/mL) essentially as described previously (O'Brien et al., 2011). For L. monocytogenes experiments, mice were stimulated once with 1 μl of STAg diluted in 200 ul PBS via retro-orbital i.v injection 24 hours prior to bacterial infection. Control mice were mock-stimulated with PBS.

rPRF.

Purified recombinant his-tagged T. gondii profilin (rPRF) was a kind gift from F. Yarovinsky or was purified similar to methods described (Yarovinsky et al., 2005). Mice were stimulated with 100 ng of rPRF in 200 μL PBS via retro-orbital i.v. injection. Control mice were mock-stimulated with PBS. For L. monocytogenes experiments, mice were stimulated once with rPRF or PBS at either 24 hours pre-4 hrs pre- or 4 hours post-bacterial infection as indicated. For cytokine analysis, mice were stimulated once with rPRF or PBS and spleens and blood were collected 2 or 24 hours later. For flow cytometry experiments, mice were stimulated once with rPRF or PBS and spleens and blood were collected 4 hours later.

Cytokine Analysis.

Blood was collected from mice via the lateral tail vein 2 or 24 hours post stimulation with rPRF as indicated. Serum was frozen in aliquots at −80° C. and then analyzed using a Mouse Inflammation Cytometric Bead Array kit (BD Biosciences, San Jose, Calif.) according to the manufacturer's instructions.

Flow Cytometry.

Spleens were dissociated by mechanical disruption and digested with collagenase/dispase (20 μg/mL, Roche, Indianapolis, Ind.) and DNAse I (300 μg/mL, Roche) for 30 minutes at 37° C. and passed through a 70 μm cell strainer (BD Biosciences, San Jose, Calif.). Heparinized blood was collected via cardiac puncture then mixed with an equal volume of 6% dextran (MP Biomedicals, Solon, Ohio). RBCs were allowed to sediment for 45 minutes at 4° C. then PMN and PBMCs were collected from the upper fraction. Remaining RBCs were lysed with ammonium chloride. Cells were stained at 4° C. in PBS with Live/Dead Violet Fixable Stain kit (Invitrogen, Carlsbad, Calif.?), washed, then stained with antibodies in PBS with 0.5 mM EDTA, 0.2% BSA, 0.09% azide, and 2% normal rat serum (Jackson ImmunoResearch, West Grove, Pa.). Anti-mouse CD11b (M1/70) PerCpCy5.5 (eBioscience, San Diego, Calif.), anti-mouse Ly6C (AL-21) PE, anti-mouse CCR2 (475301) Fluorescein (R&D Systems, Minneapolis, Minn.), and anti-mouse Ly6G (1A8) PE-Cy7 or Alexa Fluor 700 antibodies were purchased from BD Biosciences except as indicated. Anti-Rat/Hamster CompBeads (BD Biosciences) stained with experimental antibodies were used to set compensation. Data were collected on an LSRII cytometer (BD Biosciences) and analyzed with FlowJo 7.6.1 (TreeStar, Ashland, Oreg.).

In Vivo Depletions.

All antibodies used for depletions were purchased from BioXCell (West Lebanon, N.H.). To deplete NK cells, mice were treated with the anti-NK1.1 MAb PK136 (200 μg/animal). To deplete both Ly6C^{hi} inflammatory monocytes and Ly6C^{int} Ly6G⁺ neutrophils, mice were treated with anti-Gr-1 MAb RB6-8C5 (200 μg/animal) which recognizes a common epitope shared by Ly6C and Ly6G (Fleming et al., 1993). To deplete only neutrophils, mice were treated with anti-Ly6G MAb 1A8 (250 μg/animal) (Daley et al., 2008). All depletion treatments were administered in PBS via i.p. injection beginning 48 hours prior to stimulation with rPRF, then continued every 48 (1A8) or 72 (PK136 and RB6-8C5) hours after for the duration of the experiment.

Statistical Analysis.

Graphs and statistical analysis were made using Graph Pad Prism (San Diego, Calif.). Graphs represent means and error bars represent standard deviation except where noted otherwise. Bacterial burden data and cytokine levels were analyzed with the two-tailed student's t-test, and survival data were analyzed using the Log-rank (Mantel-Cox) method. p-values are represented by asterisks in figures as follows: *p<0.05, **p<0.01, ***p<0.001. We consider all p-values <0.05 to be significant. Data with no statistical significance are marked N/S.

### Results

Chronic Infection with T. gondii Confers Resistance Against L. monocytogenes Infection.

Previous research has shown that when challenged with a L. monocytogenes infection, T. gondii infected mice had greater survival or delayed time to death relative to uninfected animals (Ruskin and Remington, 1968). Further experiments showed that this protective effect was not transferable in the serum, and thus was likely a cell mediated response (Ruskin et al., 1969). Although the specific bacterial burdens were not determined for the animals in these studies, early innate control of L. monocytogenes replication generally correlates well with severity of infection in mice: animals that maintain low bacterial numbers generally go on to clear the infection, whereas failure of innate immunity is associated with high numbers of bacteria, overwhelming sepsis and inevitable death.

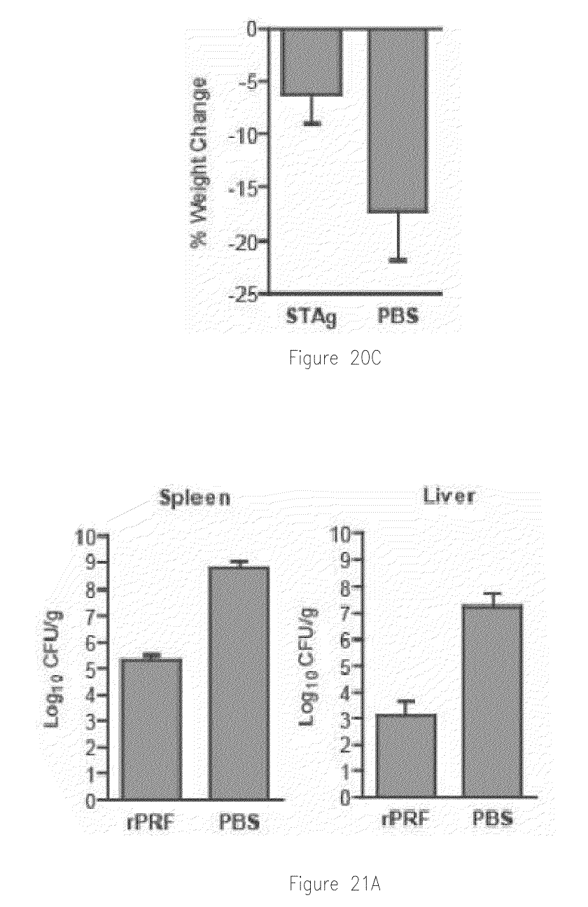

To test if the enhanced survival of T. gondii infected mice could be explained due to an early innate control of L. monocytogenes replication, C57BL/6 mice were infected with 250 Pru T. gondii tachyzoites via i.p. injection. Control animals were not infected. On day 36 post T. gondii infection, the mice were infected with a lethal inoculum (6×10⁴ CFU/animal) of L. monocytogenes EGD. 72 hours later the mice were euthanized, and the number of viable L. monocytogenes bacteria in the spleens and livers were determined. T. gondii-infected mice had an average of 5.85 log₁₀CFU/g in the spleens, a significant 3.6-log₁₀ reduction (p<0.0001) compared to uninfected controls with an average burden of 9.48 log₁₀CFU/g (FIG. 20A). T. gondii infected mice also had a 4.5-log₁₀ reduction (p<0.0001) in the livers: 4.29 log₁₀CFU/g versus 8.81 log₁₀CFU/g for uninfected controls (FIG. 20A). Mice with bacterial burden less than 6-log₁₀CFU/g in the spleen at 72 hours post infection typically remain asymptomatic and survive L. monocytogenes infection, whereas those with higher bacterial burdens typically succumb to infection. These results suggest that survival of T. gondii infected mice reported previously (Ruskin and Remington, 1968) could be explained by reduced numbers of L. monocytogenes bacteria.

Stimulation with STAg Reduces Bacterial Burden Following L. monocytogenes Infection.

Previous work with influenza virus (O'Brien et al., 2011) had shown that the protective effects of T. gondii infection could be replicated by treating mice with soluble T. gondii antigens (STAg), a non-infectious lysate of soluble material from sonicated T. gondii tachyzoites. STAg contains many T. gondii proteins, including profilin (Yarovinsky et al., 2005), and previous work has shown that STAg can stimulate immune responses similar to those induced by live parasites, including induction of IL-12, TNF-α, IFN-γ, IL-1β, IL-10, and MCP-1/CCL2 in vivo or in in vitro (Del Rio et al., 2004; Grunvald et al., 1996; Li et al., 1994). Consistent with these data, increased levels of IL-12, TNF-α, IFN-γ, and CCL2 in the serum of STAg-stimulated mice were observed within 24 hours (data not shown). Based on these data, it was hypothesized that STAg treatment may be sufficient to reduce bacterial burdens of L. monocytogenes infected mice as well as chronic T. gondii infection.

To test this hypothesis, STAg was prepared from tissue culture grown PruΔ T. gondii tachyzoites and then used in a L. monocytogenes challenge model. Mice treated with 1 μL of STAg 24 hours prior to infection with L. monocytogenes had approximately 2.5 log₁₀ reductions in bacterial burden in the spleens (p<0.0001) and 3.75 log₁₀ reductions in the liver (p=0.0003) compared to PBS-treated controls (FIG. 20B). These effects were similar to the reduction in bacterial burdens in T. gondii infected mice.

To determine if the protective components in STAg were protein or other molecules such as RNA or DNA, STAg was subjected to proteinase K digestion. Unlike untreated STAg, proteinase K-digested STAg did not reduce bacterial burdens in L. monocytogenes infected mice (data not shown), which suggested that the protective component(s) were protein. Next, to identify the specific protein(s), STAg was subjected to a series of biochemical fractionation steps including ammonium sulfate (AS) precipitation and anion exchange chromatography, and then assayed the fractions for protective activity in mice. This approach identified two fractions, an AS cut containing the proteins which remained soluble at AS concentrations >60% (data not shown), and anion exchange chromatography Fraction #3 (data not shown). Then these fractions were subjected to western blotting with available antibodies against several T. gondii proteins, including antibodies against the TLR11 ligand T. gondii profilin (TgPRF). Using this method, profilin was found to be present in both the “>60%” and “Fraction #3” active fractions.

Recombinant T. gondii Profilin is Sufficient to Reduce Bacterial Burden and Enhance Survival Following L. monocytogenes Infection.

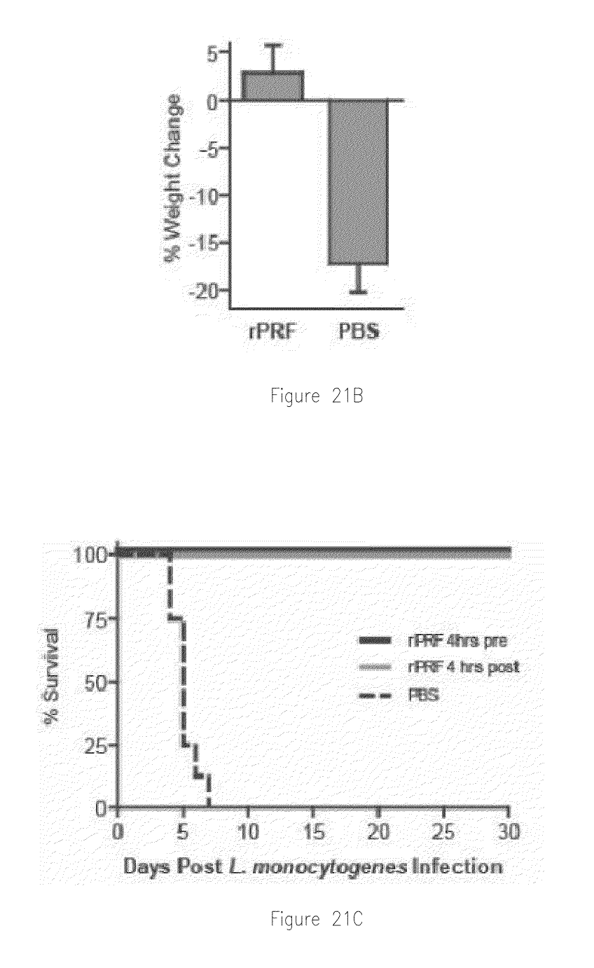

TgPRF is an parasite actin-binding protein involved in parasite gliding motility, host cell invasion and egress, and is known for inducing IL-12 production through stimulation of TLR11 expressed on CD8α⁺ DCs (Plattner et al., 2008; Yarovinsky et al., 2005). Although profilin-like proteins are found in all eukaryotes, TgPRF's ability to stimulate TLR11 is due to a parasite-specific acidic loop on the protein (Kucera et al., 2010). In order to determine if TgPRF was sufficient to confer protection in the L. monocytogenes challenge model, mice were stimulated with purified recombinant N-terminal his-tagged TgPRF (rPRF). Mice stimulated with 100 ng rPRF 4 hours prior to L. monocytogenes infection had a significant 3.4 log₁₀ reduction in bacterial burdens in the spleen (p<0.0001) and a 4.1 log₁₀ reduction in the liver (p<0.0001) compared to PBS-treated animals (FIG. 21A). rPRF-treated mice also did not show clinical symptoms of L. monocytogenes infection (ruffled fur, hunched posture) or weight loss (FIG. 21B), in contrast to PBS-treated controls which lost significantly more (17.23%) of their starting weight by 72 hours post infection (p=0.0003). rPRF stimulation was similarly effective when given 24 hours prior to or 4 hours post L. monocytogenes infection (data not shown).

Next, the long-term survival of rPRF- and PBS-treated L. monocytogenes infected mice was followed (FIG. 21C). As expected, all (8/8) PBS-treated mice rapidly succumbed to L. monocytogenes infection with 7 days, with the majority of mice succumbing by day 5. In contrast, 100% (8/8 for each group) of mice stimulated with rPRF 4 hours prior to, or 4 hours after, L. monocytogenes infection survived for 30 days, at which point the experiment was terminated (p<0.0001). None of the rPRF-treated mice experienced any clinical signs of L. monocytogenes disease (such as ruffled fur, hunched posture) at any point during the study. These results suggest that rPRF-stimulation is sufficient to reduce bacterial burdens and confer a long-term survival advantage during L. monocytogenes infection, and that rPRF-stimulation is also sufficient as a post-exposure therapeutic.

Stimulation with T. gondii PRF Induces Production of IFN-v, MCP-1 and TNF-α.

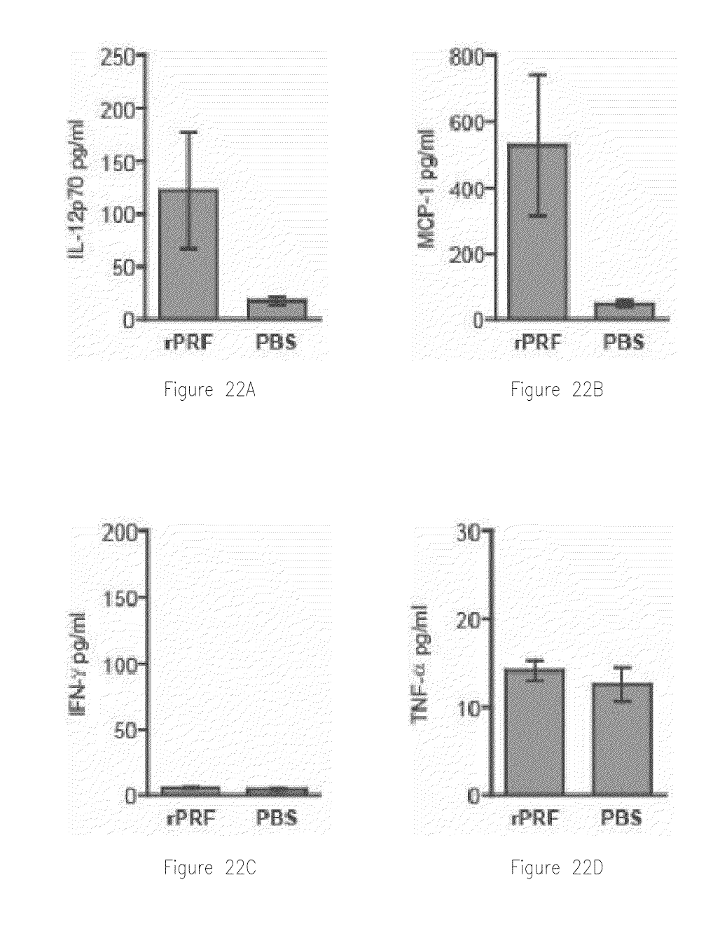

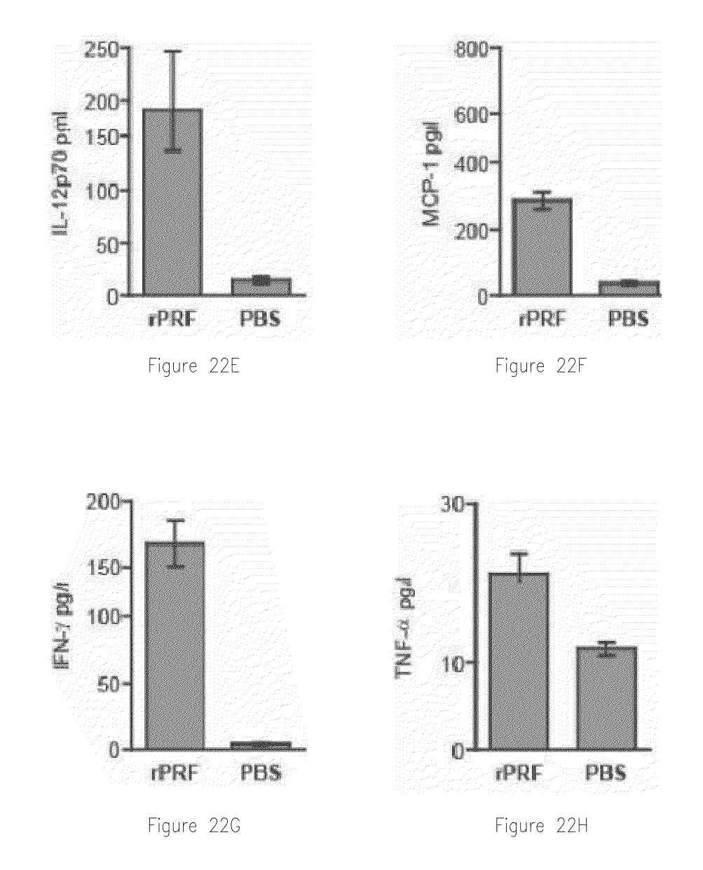

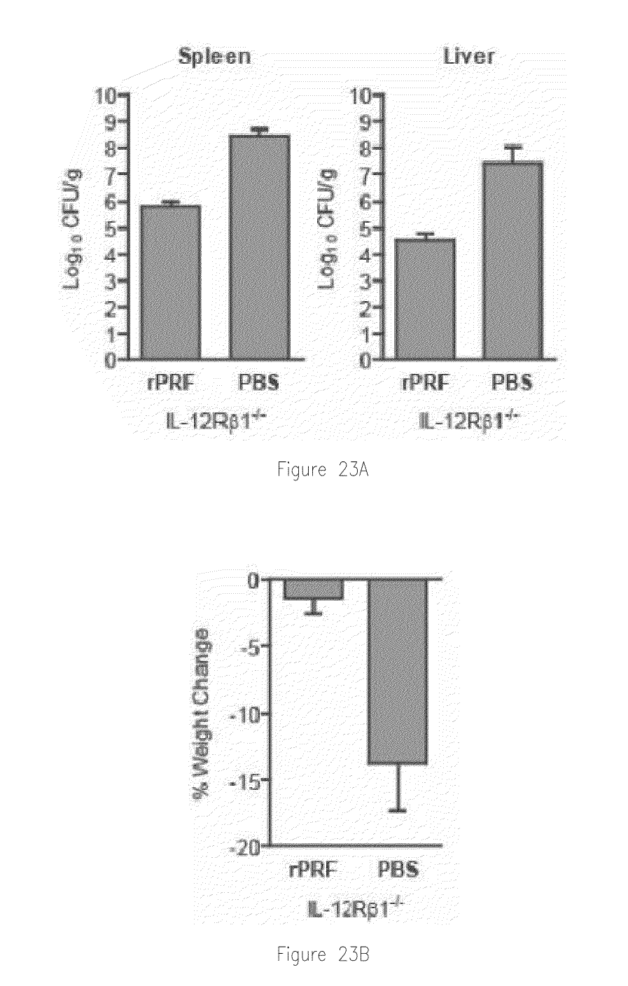

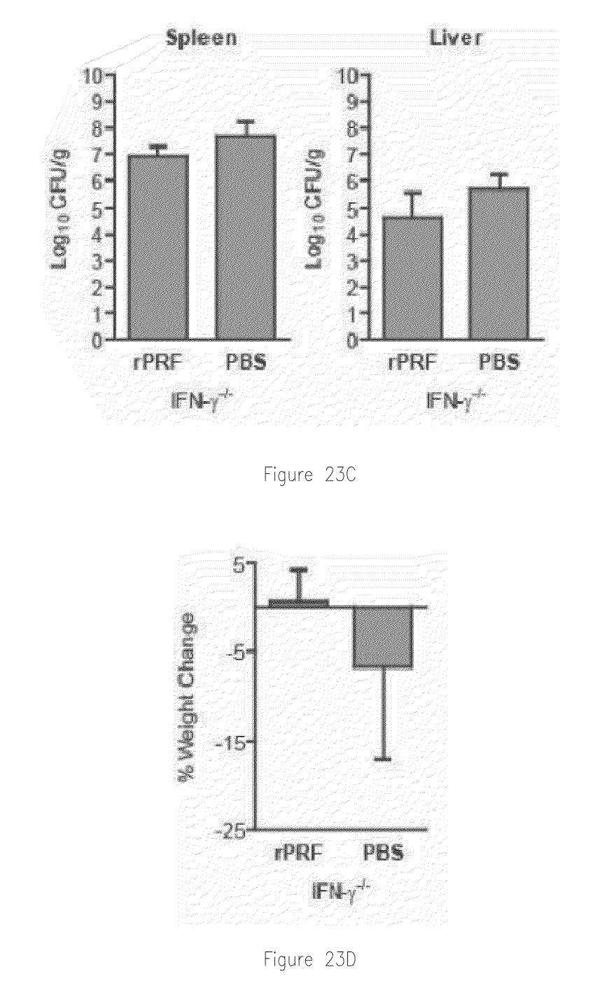

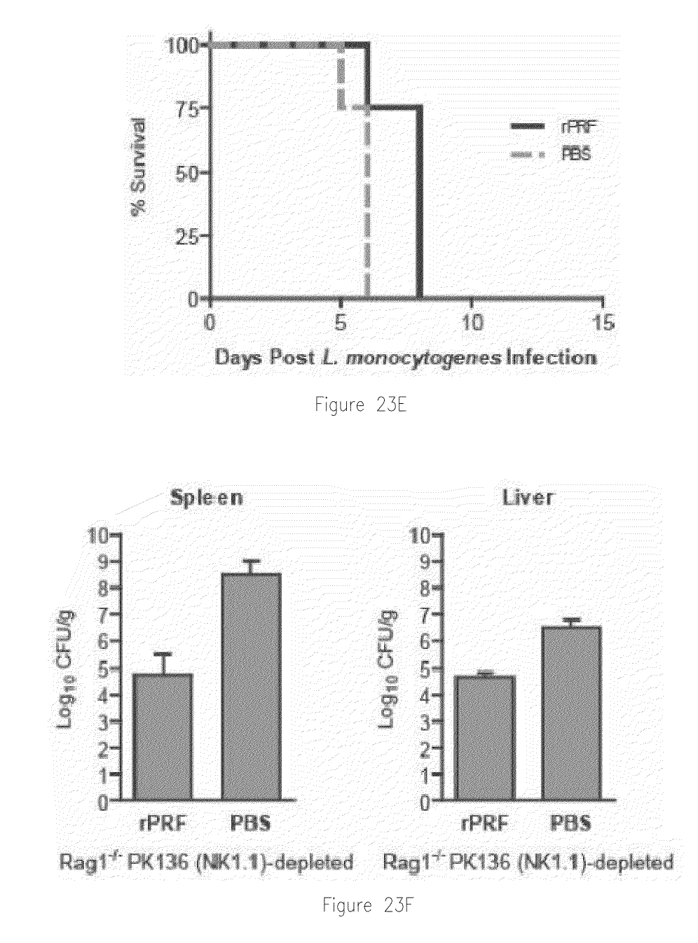

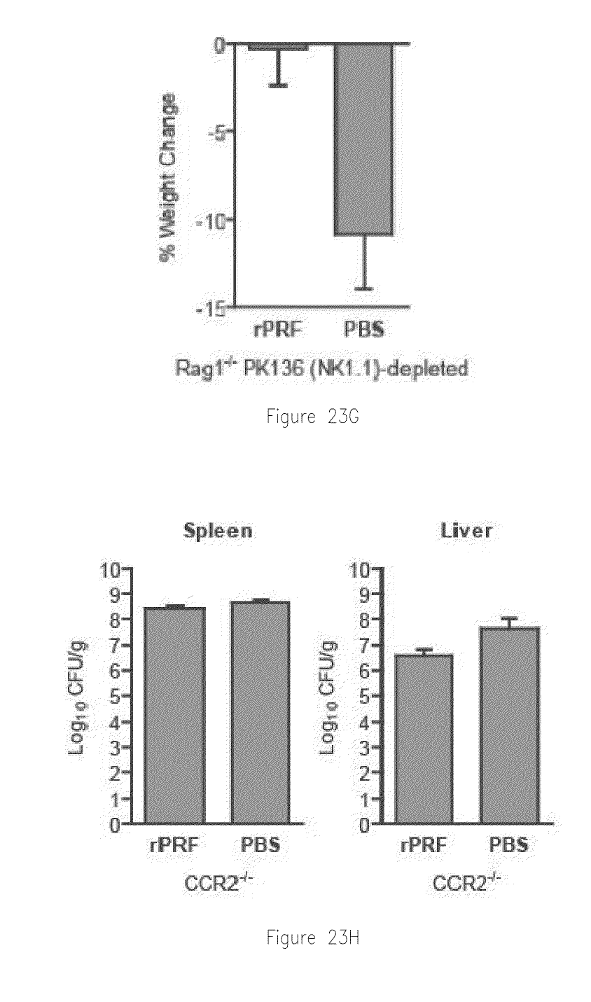

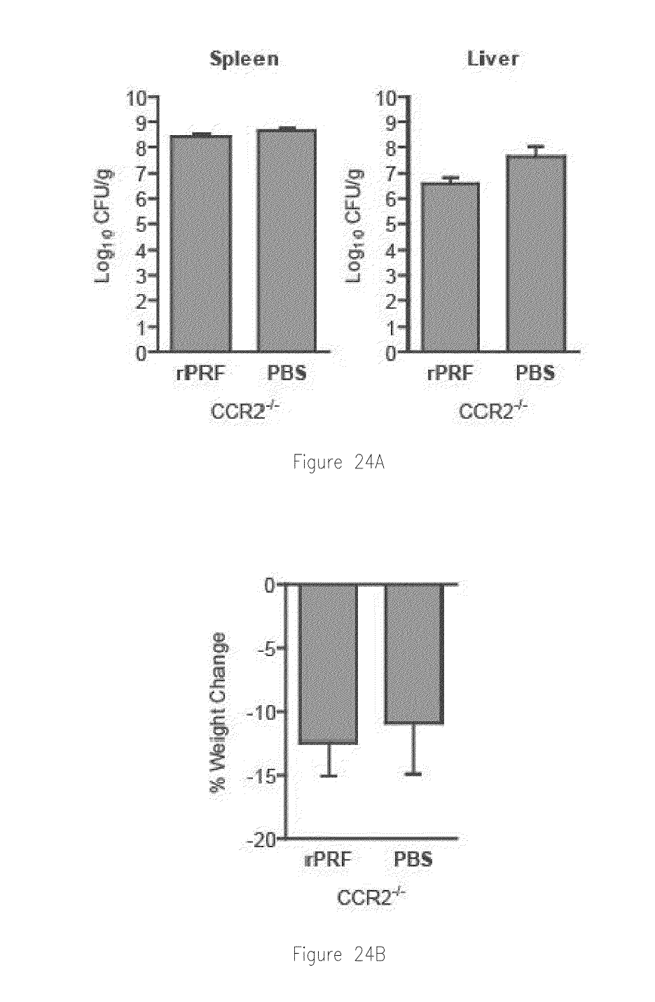

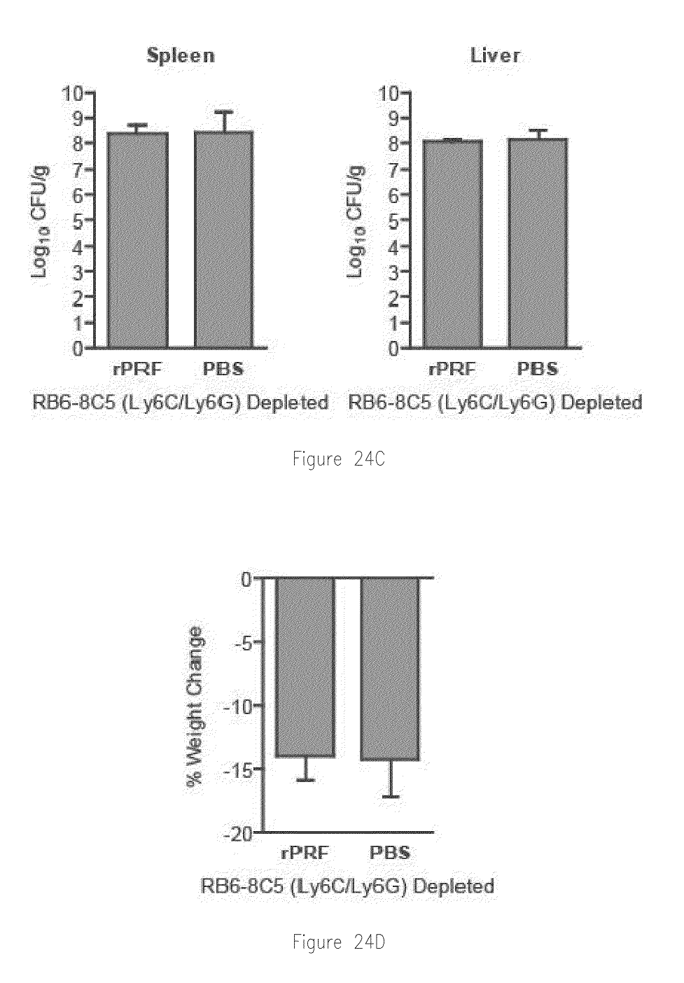

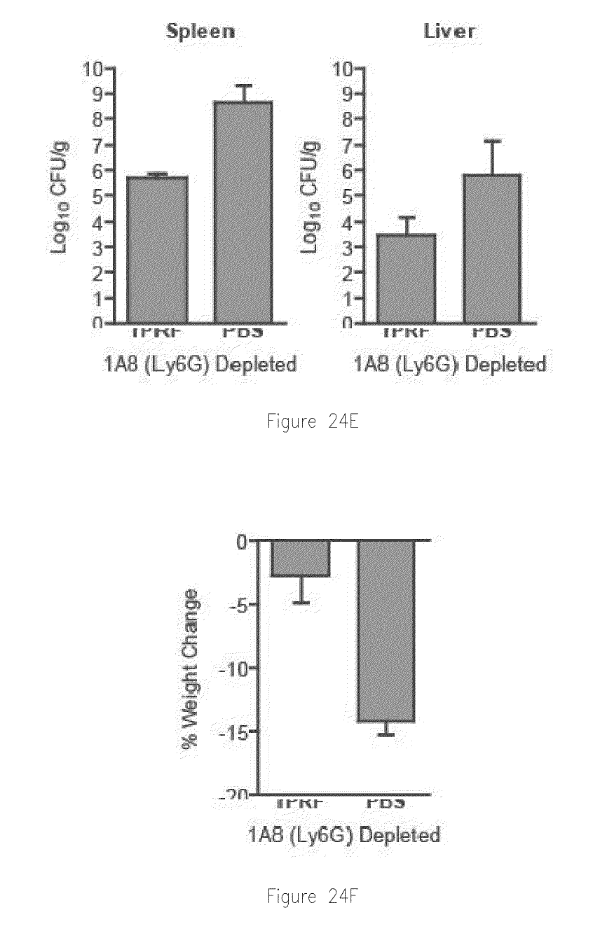

Although STAg has been shown to induce cytokines and chemokines including IL-12, TNF-α, IFN-γ, IL-1β, IL-10, and MCP-1/CCL2 (Grunvald et al., 1996; Li et al., 1994), TgPRF has not been shown to induce production of any other cytokines or chemokines other than IL-12 (Yarovinsky et al., 2005). Therefore, in order to determine if TgPRF could induce production of other anti-listerial cytokines, mice were stimulated with rPRF, and serum collected 2 or 24 hours later. As expected, significant IL-12 production was observed in response rPRF stimulation at both 2 (FIG. 22A) and 24 hours (FIG. 23). Significant production of MCP-1/CCL2 was also observed at 2 and 24 hours (FIGS. 22B and F). Significant amounts of IFN-γ and TNF-α were not detected at 2 hours (FIGS. 22C and D), but were significantly elevated by 24 hrs (FIGS. 22G and H).

T. gondii PRF-Induced IL-12 Signaling is not Required for Reducing Bacterial Burden.

IL-12 mediates defenses against T. gondii by inducing IFN-γ production from NK and T cells, which in turn helps to activate macrophage effector functions, enhancing antigen presentation, and by promoting the differentiation of Th1 cells (Sher et al., 2003). IL-12 plays a similar and critical role in L. monocytogenes infections. IL-12p35 deficient mice and mice treated with neutralizing anti-IL-12 antibody have defective IFN-γ responses and are more susceptible to high dose L. monocytogenes infection (Brombacher et al., 1999; Tripp et al., 1994; Wagner et al., 1994) and treatment of L. monocytogenes infected mice with exogenous recombinant IL-12 has been shown to reduce bacterial burdens in one study (Wagner et al., 1994). Therefore, rPRF induced IL-12 signaling may be required for the protective effects of rPRF and to reduce bacterial burden in L. monocytogenes infected mice. In order to determine if IL-12 signaling was required, IL-12Rβ1 deficient (IL-12Rβ1^{-/-}) mice were stimulated with rPRF, infected with an appropriate lethal dose (1×10⁴ CFU/animal) of L. monocytogenes bacteria four hours later, and then bacterial burdens in the spleens and livers were determined at 72 hours post infection. Surprisingly, rPRF-treated IL-12Rβ1^{-/-} mice had significant 2.6 and 2.8-log₁₀ reductions in bacterial burdens in the spleen (p=0.0001) and livers (p=0.0017) compared to PBS-treated controls (FIG. 23A). rPRF-treated IL-12Rβ1^{-/-} mice also did not show clinical symptoms of L. monocytogenes infection (ruffled fur, hunched posture) and only mild weight loss (1.45%), in contrast to PBS-treated controls which lost significantly more (13.76%) weight (p=0.0006) (FIG. 23B). These data suggest that IL-12 signaling was not required for rPRF-induced protection against L. monocytogenes infection.

T and NK Cell Derived IFN-γ is not Required for T. gondii Profilin Induced Protection.

IFN-γ is a critical mediator of innate defenses against both L. monocytogenes (Buchmeier and Schreiber, 1985; Harty and Bevan, 1995) and T. gondii (Suzuki et al., 1988). In previous work with STAg and influenza virus, it was observed that STAg-induced IFN-γ was required to mediate protection against influenza virus (O'Brien et al., 2011). In order to determine if IFN-γ played a similar role in rPRF-induced protection against L. monocytogenes, IFN-γ deficient (IFN-γ^{-/-}) mice were stimulated with rPRF, infected with an appropriate lethal dose (about 200 CFU/animal) of L. monocytogenes bacteria four or twenty four hours later, and bacterial burdens in the spleens and livers were determined at 72 hours post infection. In multiple experiments, rPRF-treated IFN-γ^{-/-} mice consistently had slight (ranging from 0.6 to 1.4-log₁₀) reductions in bacterial burdens in both the spleen (p=0.0094) and liver (p=0.0111) compared to PBS-treated controls (FIG. 23C). Despite high bacterial burdens, rPRF-treated IFN-γ^{-/-}-mice did not show clinical symptoms of L. monocytogenes infection (ruffled fur, hunched posture) and tended to experience less weight loss than PBS-treated controls at 72 hours post infection, (p=0.0190) (FIG. 23D). However, 100% of rPRF-treated IFN-γ^{-/-} mice did succumb to L. monocytogenes infection within eight days, although at a significantly delayed rate relative to PBS-stimulated animals (p=0.0023) (FIG. 23E). These results suggest that IFN-γ is at least partially required for rPRF-induced protection against L. monocytogenes.

The major sources of IFN-γ are T cells and NK cells. NK1.1+ cells are the critical source of IFN-γ for early defense against T. gondii (Denkers et al., 1993; Hunter et al., 1994), and for STAg-induced protection against influenza virus (O'Brien et al., 2011). Similarly, the majority of IFN-γ during early L. monocytogenes infection is also produced by NK1.1⁺ cells (Dunn and North, 1991; Thale and Kiderlen, 2005; Tripp et al., 1993). However, T cells can also produce IFN-γ in early responses to T. gondii (Gazzinelli et al., 1996) and early in L. monocytogenes infection, albeit to a lesser extent and in a manner not normally required for innate defenses (Carrero et al., 2006; Hiromatsu et al., 1992; Thale and Kiderlen, 2005). In order to determine if either NK1.1⁺ or T cells were a required source(s) of rPRF-induced IFN-γ for protection against L. monocytogenes, mice deficient in both T and NK cells were created by treating Rag1 deficient (Rag1^{-/-}) mice with 250 μg PK136 (anti-NK1.1) depleting monoclonal antibody beginning 48 hours prior to rPRF stimulation. In contrast to IFN-γ^{-/-} mice, rPRF-stimulation was highly effective against L. monocytogenes in Rag1^{-/-} NK1.1-depleted mice. rPRF-treated mice had a 3.7 log₁₀ reduction in bacterial burden in the spleen (p<0.0001), and 1.8 log₁₀ reduction in the liver (p<0.0001), compared to PBS-treated controls (FIG. 23F). rPRF-treated Rag1^{-/-}αNK1.1-depleted mice also did not show clinical symptoms of L. monocytogenes infection (ruffled fur, hunched posture) or weight loss at 72 hours post infection (p=0.0002) (FIG. 23G). Similar results were observed with rPRF treatment in singly deficient Rag1^{-/-} or WTαNK1.1-depleted mice (data not shown). These data suggest that although IFN-γ is partially required for rPRF-induced reduction in bacterial burdens and survival, neither T nor NK cells are likely a required source.

Ly6C^{hi} CCR2⁺ Inflammatory Monocytes and Ly6C^{int} Ly6G⁺ Neutrophils are Recruited in Response to rPRF.

During L. monocytogenes infection, MCP-1 and MCP-3 signals promote emigration of TipDC precursors, Ly6C^{hi} inflammatory monocytes, out of the bone marrow and into circulation in a CCR2-dependent manner. Because rapid, significant increases in serum levels MCP-1 were observed in rPRF-stimulated mice (FIGS. 22B and F) and because T. gondii infection is also known to elicit a population of Ly6C^{hi} monocytes, it was determined if stimulation with T. gondii profilin promoted emigration of Ly6C^{hi} monocytes. When blood and spleens from mice stimulated with rPRF or PBS were analyzed by flow cytometry, an approximate 8-fold increase in the number of CD11b⁺ Ly6C^{hi} monocytes in blood (p=0.0203) and an approximate 4-fold increase in the spleens (p<0.0001) was observed within four hours (FIGS. 24A and B). Ly6C^{hi} monocyte populations expressed CCR2 (FIG. 5E), consistent with an inflammatory monocyte TipDC precursor population. Significant 4-fold and 2-fold increases in a Ly6C^{int} Ly6G⁺ neutrophil population were observed in the blood (p=0.0083) and spleens (0.0067) of rPRF-stimulated mice (FIGS. 24C and D). The Ly6C^{int} Ly6G⁺ neutrophil population did not express CCR2 (FIG. 24E).

Ly6C^{hi} CCR2⁺ Inflammatory Monocytes are Required for rPRF-Induced Defenses.

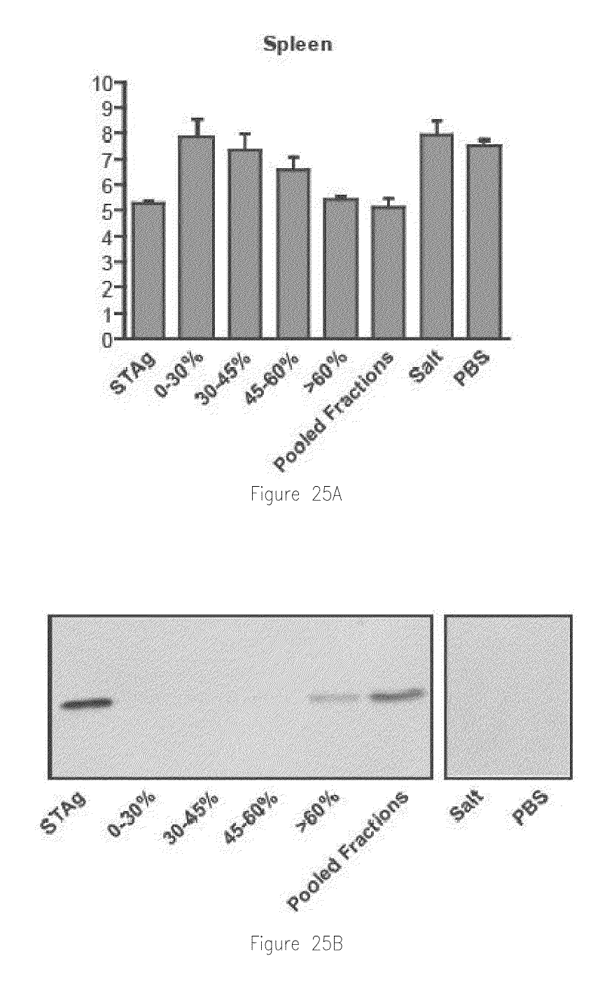

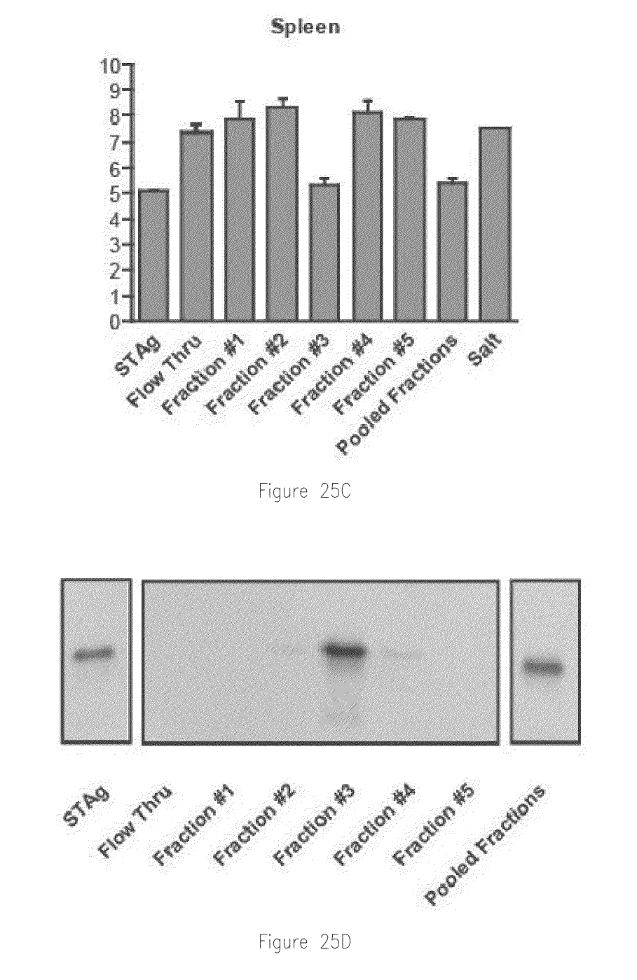

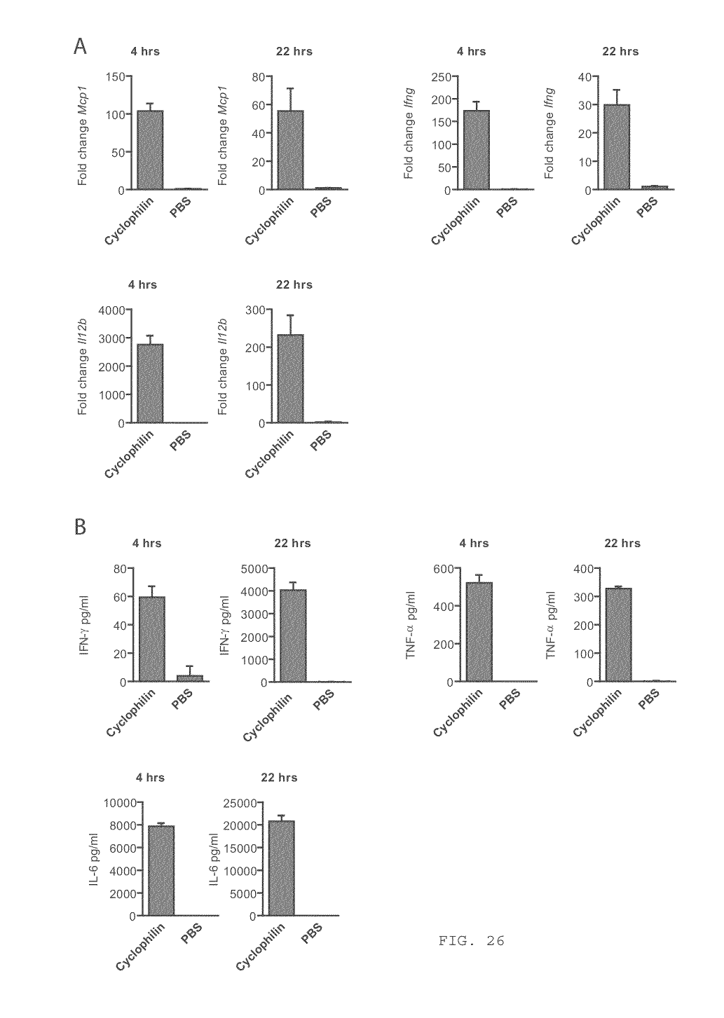

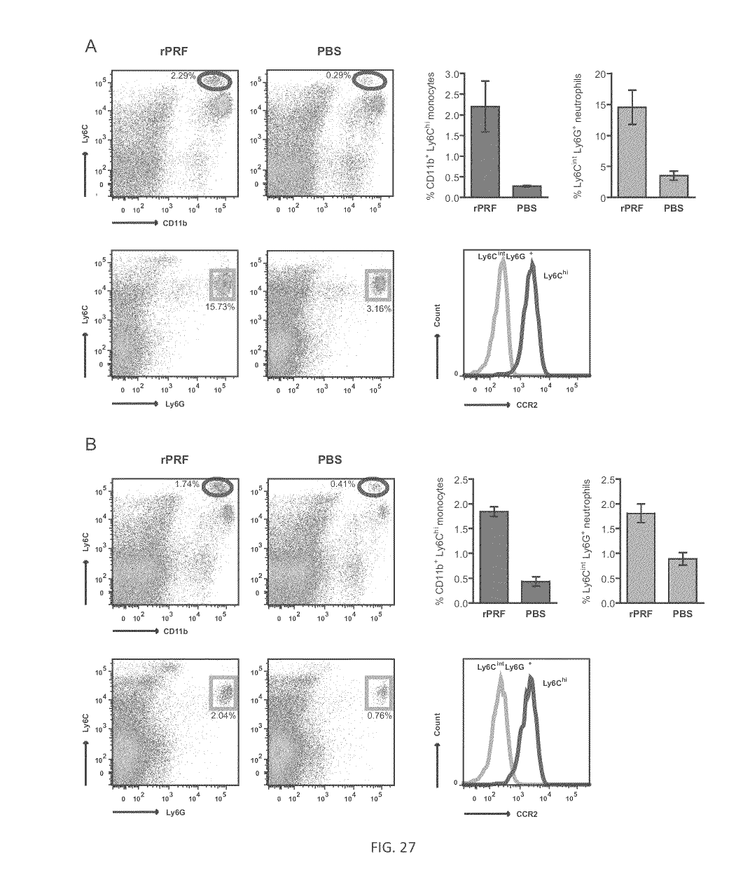

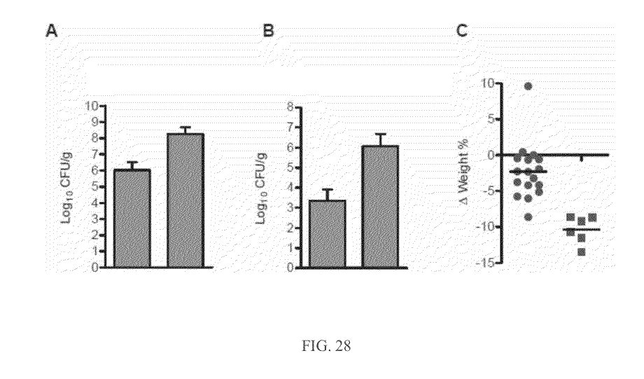

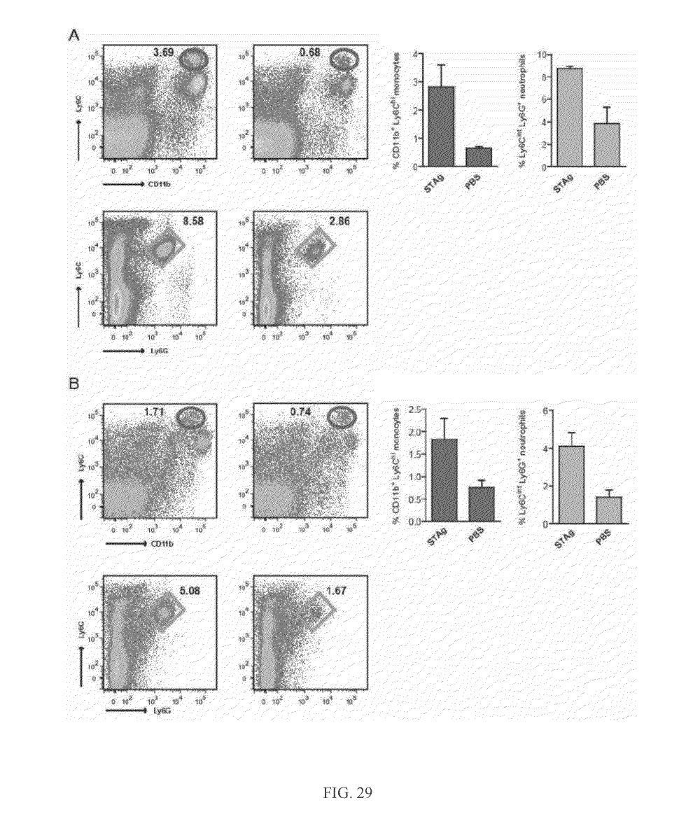

Ly6C^{hi} CCR2⁺ monocyte emigration from the bone marrow into circulation is CCR2-dependent (Serbina and Pamer, 2006). Therefore, in order to determine if Ly6C^{hi} CCR2⁺ cells recruited in response to rPRF contributed to protection, CCR2 deficient (CCR2^{-/-}) mice were stimulated with rPRF 4 hours prior to infection with a lethal (8×10³ CFU) dose of L. monocytogenes. rPRF-stimulated CCR2^{-/-} mice did not have large reductions in bacterial burdens in the spleen (0.2 log₁₀) or liver (1.0 log₁₀) at 72 hours post infection compared to PBS-stimulated controls (FIG. 25A). Although the reductions were statistically significant (p=0.0138 and p=0.0019), they are not likely biologically relevant given the overall high burdens. In addition, both groups showed clinical signs of disease (ruffled fur and hunched posture) and experienced weight loss equal to PBS-treated controls (p=0.5214) (FIG. 25B).

CCR2^{-/-} mice do not lack Ly6C^{hi} CCR2⁺ monocytes, rather they are only defective in their migration out of the bone marrow and into circulation. CCR2^{-/-} mice have diminished levels or circulating Ly6C^{hi} monocytes but increased numbers in the bone marrow at rest, and large numbers of activated TNF-α producing Ly6C^{hi} monocytes accumulate in the bone marrow of CCR2^{-/-} mice during L. monocytogenes infection (Jia et al., 2008; Serbina and Pamer, 2006). Thus, the small reduction in bacterial burden that was observed, mainly in the livers, of rPRF-stimulated CCR2^{-/-} mice could potentially still be dependent on Ly6C^{hi} CCR2⁺ monocytes, either by activation of a limited number of cells in circulation, or via soluble cytokines such as TNF-α produced by those cells restricted to the bone marrow. Therefore, in order to significantly deplete Ly6C^{hi} CCR2⁺ monocytes, mice were treated with the anti-Gr-1 MAb RB6-8C5, which recognizes a common epitope shared by Ly6C and Ly6G (Fleming et al., 1993). For Gr-1 (Ly6C/Ly6G) depletions, mice were treated with RB6-8C5 MAb beginning 48 hours prior to rPRF or PBS stimulation, then the animals were infected with an appropriate lethal dose (about 200 CFU) of L. monocytogenes 4 hours later. It was consistently observed that rPRF-stimulation did not offer any protection in RB6-8C5 depleted mice. There were no significant differences in bacterial burdens between rPRF- and PBS-stimulated mice in either the spleens (p=0.9172) or livers (p=0.7096) (FIG. 25C), and both groups showed clinical signs of disease and experienced equal weight loss (p=0.8926) (FIG. 25D) by 72 hours post infection.

Because RB6-8C5 significantly depletes Ly6G⁺ neutrophils as well as Ly6C^{hi} monocytes, the Ly6G specific MAb 1A8 (Daley et al., 2008) was used to deplete mice to establish the relative contribution of Ly6G⁺ cells. Mice were depleted with 1A8 MAb beginning 48 hours prior to rPRF-stimulation, and then infected with an appropriate dose (8×10³ CFU) of L. monocytogenes 4 hours later. In contrast to CCR2^{-/-} and RB6-8C5-depleted mice, 1A8-depleted rPRF-stimulated animals were consistently protected against L. monocytogenes infection as assessed by bacterial burdens and weight loss (FIGS. 25E and F). rPRF-stimulation reduced bacterial burdens in the spleens of 1A8-depleted mice by at least 3.0 log₁₀ (p=0.0001) and in the livers by at least 2.3 log₁₀ (p=0.0226) at 72 hours post infection, although bacterial burdens in livers were highly variable among mice. rPRF-stimulated 1A8-depleted also did not show clinical symptoms of L. monocytogenes infection (ruffled fur, hunched posture) or weight loss in contrast to PBS-stimulated controls which lost significantly more weight (p<0.0001) (FIG. 6F). Together, these results indicate that although rPRF stimulation results in a large influx of Ly6C^{int} LyG⁺ neutrophils into the blood and spleen, these cells are largely dispensable for rPRF induced protection and reduction of bacterial burden in the spleen and liver. And although Ly6G⁺ neutrophils may have a small contribution in the liver following rPRF-treatment in some mice, CCR2-dependent recruitment of Ly6C^{hi} CCR2⁺ inflammatory monocytes plays the central and essential role in rPRF-induced bacterial clearance and protection against L. monocytogenes.

Chronic T. gondii infection or stimulation with STAg provided a resistance against L. monocytogenes bacterial infection by reducing bacterial burdens in the major sites of bacterial replication, the spleen and liver. Stimulation with purified recombinant T. gondii profilin was sufficient to induce this protection, but it functions independent of IL-12 and of T- and NK-derived IFN-γ. Most importantly, stimulation by T. gondii profilin resulted in production of MCP-1, which results in the trafficking of Ly6C^{hi} CCR2⁺ inflammatory monocytes into the blood and spleen, and that CCR2-dependent recruitment of these cells is essential to TgPRF-induced anti-listerial response. These results identify the TgPRF as the first known T. gondii factor to promote recruitment of Ly6C^{hi} CCR2⁺ inflammatory monocytes, and highlight the potential use of TgPRF and other T. gondii proteins as immunotherapeutics for bacterial and other infections.

### REFERENCES

Alexander et al., Chem. Immunol., 70:81 (1998).
Aliberti et al., Nat. Immunol., 4:485 (2003).
Amani et al., Eur. J. Immunol., 30:1646 (2000).
Amante et al., The Journal of Immunology, 185:3632 (2010).
Baptista et al., Infect. Immun., 78:4033 (2010).
Belnoue et al., Parasite Immunology, 30:544.
Boucher, Clin Infect Dis., 48:1 (2009).
Brombacher et al., Int. Immunol., 11:325 (1999).
Bucher et al., Int. J. Parasitol (2010).
Buchmeier et al., Proc. Natl. Acad. Sci. USA, 82:7404 (1985).
Carrero et al., J. Exp. Med., 203:933 (2006).
Charest et al., J. Immunol., 165:2084 (2000).
Clark et al., Malaria Journal, 2:6 (2003).
Czuprynski et al., J. Immunol., 152:1836 (1994).
Daley et al., J. Leukoc. Biol., 83:64 (2008).
Del Rio et al., J. Immunol., 172:6954 (2004).
de Souza et al., Parasitology:1 (2009).
Denkers et al., Clin. Microbiol. Rev., 11:569 (1998).
Denkers et al., J. Immunol., 150:517 (1993).
Dubey et al., Int. J. Parasitol, 38:1257 (2008).
Dubey, Int. J. Parasitol, 28:1019 (1998).
Dunn and North, Infect. Immun., 59:2892 (1991).
Fauconnier et al., The Journal of Immunology, 188:1905 (2012).
Frankel et al., Proc. Natl. Acad. Sci., 104:10181 (2007).
Franklin et al., Proc. Natl. Acad. Sci. USA, 108:3689 (2011).
Gazzinelli et al., J. Immunol., 153:2533 (1994).
Gazzinelli et al., J. Immunol., 157:798 (1996).
Gentry et al., J. Infect. Dis., 123:22 (1971).
Grau et al., J. Immunol., 137:2348 (1986).
Grau et al., J. Infect. Dis., 187:461 (2003).
Grau et al., Proc. Natl. Acad. Sci. USA, 86:5572 (1989).
Grau et al., Science, 237:1210 (1987).
Grunvald et al., Infect. Immun., 64:2010 (1996).
Harty and Bevan, Immunity, 3:109 (1995).
Hansen et al., J. Immunol., 178:5779 (2007).
Hague et al., The Journal of Immunology (2011).
Hermsen et al., Mol. Biochem. Parasitol, 118:247 (2001).
Hermsen et al., Parasitology, 114(Pt 1):7 (1997).
Hiromatsu et al., J. Exp. Med., 175:49 (1992).
Hochman et al., J. Neuroparasitology, 3 (2012).
Hunt et al., Trends Parasitol, 26:272 (2010).
Hunter et al., Infect. Immun., 62.2818 (1994).
Idro et al., JAMA, 297:2232 (2007).
Jia et al., J. Immunol., 180:6846 (2008).
John et al., Expert Rev. Anti. Infect. Ther., 8:997 (2010).
Jones et al., J. Virol., 80:11960 (2006).
Kihara et al., Trop. Med. Int. Health, 11:386 (2006).
Krishna S., Current Opinion in Infectious Diseases, 25:484 (2012).
Li et al., Infect. Immun., 62:3434 (1994).
Mahmoud et al., Nature, 263:56 (1976).
Martins et al, PLoS One, 7:e32046 (2012).
Mason et al., J. Immunol., 172:3704 (2004).
Medana et al., Immunol. Cell. Biol., 79:101 (2001).
Medana et al., Int. J. Parasitol., 36:555 (2006).
Microbial Threats to Health. Smolinski, M. S., Hamburg, M. A., and Lederberg, J. editors, 2003. The national academic press, Washington, D.C.
Mordue et al., Mol. Micro., 63:482 (2007).
Mordue et al., Mol. Microbiol., 63:482 (2007).
Mordue and Sibley, J. Immunol., 159:4452 (1997).
Morrell et al., Infect. Immun., 79:1750 (2011).
Morton et al., ILAR, 41:80 (2000).
Moscona, N. Eng. J. Med., 360:953 (2009).
Neyer et al., Infect. Immun., 65:1675 (1997).
Nie et al., PLoS Pathog., 5:e1000369 (2009).
Niikura et al., Int. J. Parasitol, 40:101 (2010).
Nitcheu et al., J. Immunol., 170:2221 (2003).
Normaznah et al., Malays. J. Pathol., 21:123 (1999).
O'Brien et al., J. Virol., 85:8680 (2011).
Payne et al., Mol. Microbiol., 82:1204 (2011).
Penet et al., Proc. Natl. Acad. Sci. USA, 105:1321 (2008).
Perona-Wright et al., Cell Host Microbe., 6:503 (2009).
Perrone et al., PLoS Pathogens, 4:e1000115, 1 (2008).
Pinner et al., JAMA., 275:189 (1996).
Plattner et al., Cell Host Microbe. 3:77 (2008).
Pollard et al., Infect. Immun., 77:3731 (2009).
Pollard et al., Trends Parasitol, 25:491 (2009).
Reis et al., J. Exp. Med., 186:1819 (1997).
Reis et al., PLoS Pathog., 6:e1000963 (2010).
Remington et al., Proc. Soc. Exp. Biol. Med., 131:1184 (1969).
Renia et al., Int. J. Parasitol, 36:547 (2006).
Rooney et al., Mol. Biochem. Parasitol, 176:121 (2011).
Ruskin et al., Science, 160:72 (1968).
Ruskin et al., J. Immunol., 103:252 (1969).
Scharton-Kersten et al., J. Exp. Med., 185:1261 (1997).
Scharton-Kersten et al., J. Immunol., 157:4045 (1996).
Sedegah et al., Proc. Natl. Acad. Sci. USA, 91:10700 (1994).
Serbina and Pamer, Nat. Immunol., 7:311 (2006).
Serghides et al., PLoS One, 6:e27714 (2011).
Sher et al., Immunol. Res., 27:521 (2003).
Silamut et al., Am. J. Pathol., 155:395 (1999).
Specht et al., Microbes Infect., 12:635 (2010).
Spellburg et al., Clin. Infect. Dis., 46:155 (2008).
Stevenson et al., J. Immunol., 155:2545 (1995).
Sun et al., Infect. Immun., 71:6553 (2003).
Suzuki et al., J. Immunol., 164:5375 (2000).
Suzuki et al., Science, 240:516 (1988).
Thale and Kiderlen, Immunobiology, 210:673 (2005).
Thumwood et al., Parasitology, 96(Pt 3):579 (1988).
Tripp et al., J. Immunol., 152:1883 (1994).
Tumpey et al., J. Virol., 74:6105 (2000).
Tumpey et al., J. Virol., 79:14933 (2005).
van den Steen et al., Eur. J. Immunol., 38:1082 (2008).
Van et al., Int. J. Parasitol., 37:877 (2007).
Wagner et al., Microb. Pathog., 17:175 (1994).
Ware & Kasper, L. H., Infect. Immun., 55:78 (1987).
Wilson et al., J. Neuroimmunol., 165:63 (2005).
Wong et al., AIDS, 7:299 (1993).
World Health Organization, Trans. R. Soc. Trop. Med. Hyg., 94:S1 (2000).
World Health Organization. Deaths by cause, sex and mortality stratum in WHO regions, estimates for 2002: World Health Report-2004.
Yap et al., Immunobiology, 201:240 (1999).
Yarovinsky et al., Int. J. Parasitol, 36:255 (2006).
Yarovinsky et al., Science, 308:1626 (2005).
Yuri C Martins GMZJAFLJMC. 2012. Efficacy of Different Nitric Oxide-Based Strategies in Preventing Experimental Cerebral Malaria by Plasmodium berghei ANKA. PLoS ONE 7.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent, inhibit or treat microbial pathogen infection or replication in a mammal, comprising: administering to a mammal a composition comprising an effective amount of an isolated protein having at least 80% amino acid sequence identity to SEQ ID NO:3 or SEQ ID NO:4, or a recombinant bacterial cell, recombinant eukaryotic cell or recombinant virus that expresses an isolated protein having at least 80% amino acid sequence identity to SEQ ID NO:3 or SEQ ID NO:4, or any combination thereof, or a composition comprising an effective amount of a combination of an isolated protein having at least 80% amino acid sequence identity to SEQ ID NO:3, and an isolated protein having at least 80% amino acid sequence identity to SEQ ID NO:1, SEQ ID NO:2 or SEQ ID NO:4, or a combination thereof.

2. A method to prevent, inhibit or treat microbial pathogen infection or replication in a mammal, comprising administering to a mammal that does not express TLR11, a composition comprising an effective amount of an isolated protein having at least 80% amino acid sequence identity to SEQ ID NO: 1, or a recombinant bacterial cell, recombinant eukaryotic cell or recombinant virus that expresses an isolated protein having at least 80% amino acid sequence identity to SEQ ID NO:1, or any combination thereof.

3. The method of claim 1 wherein the composition comprises a protein having at least 85% amino acid sequence identity to SEQ ID NO:1, SEQ ID NO:3 or SEQ ID NO:4.

4. The method of claim 1 wherein the composition comprises a protein having at least 90% amino acid sequence identity to SEQ ID NO: 1, SEQ ID NO:3 or SEQ ID NO:4.

5. The method of any one of claims 1 to 5 wherein the microbial pathogen is a bacterium, virus, fungus or parasite.

6. The method of claim 5 wherein the bacterium is Listeria.

7. The method of claim 5 wherein the virus is influenza virus.

8. The method of claim 5 wherein the parasite is a Plasmodium.

9. The method of claim 1 wherein the composition further comprises a pharmaceutically acceptable carrier.

10. The method of claim 1 wherein the composition is administered after the mammal is exposed to the microbial pathogen.

11. The method of claim 1 wherein the composition is administered to the mammal prior to exposure to the microbial pathogen.

12. The method of claim 1 wherein the composition is parenterally administered, intranasally administered, orally administered or intra-muscularly administered.

13. The method of claim 1 wherein the mammal is a human.

14. A composition comprising an amount of an isolated protein having at least 80% amino acid sequence identity to SEQ ID NO:3 or SEQ ID NO:4, or a combination thereof, or an isolated protein having at least 80% amino acid sequence identity to SEQ ID NO:3, and an amount of an isolated protein having at least 80% amino acid sequence identity to SEQ ID NO:1, SEQ ID NO:2 or SEQ ID NO:4, effective to inhibit or prevent microbial pathogen infection or replication, or one or more symptoms or manifestations of the microbial pathogen infection or replication, in a mammal.

15. The composition of claim 14 wherein the protein has at least 85% amino acid sequence identity to SEQ ID NO:3 or SEQ ID NO:4.

16. The composition of claim 14 wherein the protein has at least 85% amino acid sequence identity to SEQ ID NO: 1.

17. The composition of claim 15 which comprises from about 5 mg to about 50 mg of protein.

18. The composition of claim 15 further comprising an immunogen for a microbial pathogen.

19. The composition of claim 15 further comprising one or more immunogens, one or more stabilizers, one or more preservatives, or combinations thereof.
